SEQUENCE LISTING (36 sequences)

SEQ ID NO: 1 - DNA, 46 bp, synthetic construct
  source 1..46: mol_type = other DNA
gtgcgggtgccagggcgtgcccttgggctccccgggcgcgtactcc

SEQ ID NO: 2 - DNA, 46 bp, synthetic construct
  source 1..46: mol_type = other DNA
agtgccccaactggggtaacctttgagttctctcagttgggggcgt

SEQ ID NO: 3 - DNA, 46 bp, synthetic construct
  source 1..46: mol_type = other DNA
gtgcgggtgccagggcgtgcccaggggctccccgggcgcgtactcc

SEQ ID NO: 4 - DNA, 46 bp, synthetic construct
  source 1..46: mol_type = other DNA
agtgccccaactggggtaacctaggagttctctcagttgggggcgt

SEQ ID NO: 5 - DNA, 49 bp, synthetic construct
  source 1..49: mol_type = other DNA
cggccggcttgtcgacgacggcggactccgtcgtcaggatcatccgggc

SEQ ID NO: 6 - DNA, 55 bp, synthetic construct
  source 1..55: mol_type = other DNA
gtcgtggtttgtctggtcaaccaccgcggactcagtggtgtacggtacaaaccca

SEQ ID NO: 7 - DNA, 49 bp, synthetic construct
  source 1..49: mol_type = other DNA
cggccggcttgtcgacgacggcggtctccgtcgtcaggatcatccgggc

SEQ ID NO: 8 - DNA, 55 bp, synthetic construct
  source 1..55: mol_type = other DNA
gtcgtggtttgtctggtcaaccaccgcggtctcagtggtgtacggtacaaaccca

SEQ ID NO: 9 - DNA, 73 bp, synthetic construct
  source 1..73: mol_type = other DNA
tatcaccgcaagggataaatatctaacaccgcgcgtgttgactattttacctctggcggtgataatggttgca

SEQ ID NO: 10 - DNA, 61 bp, synthetic construct
  source 1..61: mol_type = other DNA
aaccatctgcggtgataaattatctctggcggtgttgacataaataccactggcggtgata

SEQ ID NO: 11 - DNA, 62 bp, synthetic construct
  source 1..62: mol_type = other DNA
acataagcacctgtaggatcgtacaggtttagcgaagaaaatggtttgttatagtcgaataa

SEQ ID NO: 12 - DNA, 141 bp, synthetic construct
  source 1..141: mol_type = other DNA
taatcaaaaaagcttaattaagatcaatttgatctacatctctttaaccaacaatatgtaagatctcaactatcgcatccgtggattaattcaattataacttctctctaacgctgtgtatcgtaacggtaacactgtaga

SEQ ID NO: 13 - DNA, 29 bp, synthetic construct
  source 1..29: mol_type = other DNA
ttgacaattaatcatcggctcgtataatg

SEQ ID NO: 14 - DNA, 52 bp, synthetic construct
  source 1..52: mol_type = other DNA
ttgttcagaacgctcggtcttgcacaccgggcgttttttctttgtgagtcca

SEQ ID NO: 15 - DNA, 47 bp, synthetic construct
  source 1..47: mol_type = other DNA
ccaattattgaagcggctaacgccgctttttttgtttctggtctccc

SEQ ID NO: 16 - DNA, 47 bp, synthetic construct
  source 1..47: mol_type = other DNA
tctaactaaaaacaccctaacgggtgttttttcttttctggtctccc

SEQ ID NO: 17 - DNA, 29 bp, synthetic construct
  source 1..29: mol_type = other DNA
  misc_feature 1..29: note = labelled at 5' end with HEX and labelled at 3' end with BHQ1
tcatccggggtcagcaccgtttctgcgga

SEQ ID NO: 18 - DNA, 25 bp, synthetic construct
  source 1..25: mol_type = other DNA
  misc_feature 1..25: note = labelled at 5' end with 6-FAM and labelled at 3' end with BHQ1
cagcccttttgcacgatggtgcgca

SEQ ID NO: 19 - DNA, 20 bp, synthetic construct
  source 1..20: mol_type = other DNA
cgcttgcgttttcccttgtc

SEQ ID NO: 20 - DNA, 19 bp, synthetic construct
  source 1..19: mol_type = other DNA
gcagccacgtagaaagcca

SEQ ID NO: 21 - DNA, 20 bp, synthetic construct
  source 1..20: mol_type = other DNA
tggtgcaacattcacatcgt

SEQ ID NO: 22 - DNA, 20 bp, synthetic construct
  source 1..20: mol_type = other DNA
agcgaaatctgtgccaactt

SEQ ID NO: 23 - DNA, 11269 bp, synthetic construct
  source 1..11269: mol_type = other DNA
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaaacgcaaaaaaccccgcccctgacagggcggggttttttcgcgtgcgggtgccagggcgtgccctcgagttctctcagttgggggcgtgctattcgatagttgttaaggtcgcggtctgacgctcagtggaacaaccatctgcggtgataaattatctctggcggtgttgacataaataccactggcggtgatactgagcacatgtactcagatccaataggaggaacaatatgcgcgcgcttgttgtgattcgtttatcgcgtgtgacggacgccaccaccagtcccgaacgccaattagaaagctgtcaacaactttgtgctcaacgtggttgggatgtggtgggagtggccgaagaccttgatgtttcgggcgctgttgacccatttgatcgcaaacgtcgtcctaatttggcgcgttggctggcctttgaagaacagcctttcgatgtaattgtcgcatatcgcgttgaccgtctgacacgttccattcgccacctgcaacaacttgtgcattgggcggaagatcataagaaattagttgtttcggcaacagaggcccattttgacaccaccacacctttcgctgctgtagttattgccctgatgggtactgtcgctcaaatggagctcgaggcaattaaggaacgcaatcgctccgcggcccactttaacattcgtgctggcaagtatcgcggcagcttacccccgtggggttatctccccacacgtgtagatggcgaatggcgcctcgtccctgatccggtccaacgcgaacgtattctggaagtctaccatcgtgtagtggataatcatgaacccttacacctcgttgctcatgatctcaatcgccgcggcgttttgagccccaaagattattttgcccaattgcagggtcgtgaaccccaaggtcgcgaatggtccgccacagccttaaaacgctcaatgatttctgaagcaatgcttggctatgcaacgttgaatggcaaaactgttcgtgatgatgatggcgcgcccttggttcgcgccgaaccaattttgacacgcgaacaattagaagctcttcgtgcagaactggttaaaacatcgcgtgccaaaccggccgttagtactccttctcttctccttcgtgtcctgttttgtgctgtctgtggcgaacctgcctataaatttgctggtggcgggcgcaaacatcctcgttatcgttgtcgtagcatgggctttcccaaacattgtggcaatggtactgttgctatggcggaatgggatgccttttgtgaagaacaagtgttagaccttctgggcgatgcagaacgcctcgaaaaggtatgggtcgccgggagcgatagcgcagtggagctggctgaagtaaatgccgaactcgttgatttaactagcttgattggatctcctgcgtatcgcgccggatcgccacaacgtgaggccttagacgcgcgcatcgctgccttagctgcccgccaggaagaactcgaaggtttggaagcgcgtcctagcggatgggaatggcgtgaaactggtcaacgctttggagattggtggcgcgaacaagatacggctgccaagaatacatggctgcgttccatgaatgtgcgtcttacctttgatgtgcgtggtggtttaacccgtacaattgattttggcgacctgcaagaatatgaacaacacctgcgtctgggatcagtcgtggagcgcttgcatacaggcatgtcataaggcatctaactaaaaacaccctaacggcaaaataataaaaaagccggattaataatctggctttttatattctctcactaccatcggcgctacggcgtttcacttctgagttctgcagcggccgctactagtatggtaccattacgccgctacgtcttccgtgccgtcttgagcatcatcctcatcatcatccgttgggggtttagcccaagtaatactcgcacgtttctcaatcggggtgccttggccgcgtccggtcgttgatttggtcaccacaattttatcaacaaacaggcccacaaatacacgtttatcatccacacttgcacggccccaccagcttttcggacctgttgggtccgcatccgcatcttccggaaaccactgatccagcggcaatttgggtgcctcggccgcctcgagctctgccagacgttcctcggcgccctgttgacgtaaggttaatgccgcttgctgtttgcgaaaatgtttacgaccgaccggaccatcatatgccccggccgcacgatcctcatacagctcttcaagggcgttcagcgcatccgcacgttcggccaccagattggcgcgctctcctgatttctccggggcttcggtaagtttaccaaaacggcgcgccgcttcccataacaatgccagggtttcctcatcaccctctgcatgacgaattttattaaagatacgctcggctacaaatttatccagcgcggccatagacacattacaagttccctcatgttggcccggcgccgagggatcaacgactttgcgacggcgacaacgatatgaatctttaattgactcctcgccacgtttggatgtcataaccgcaccgcattcacaataaagtttatccatcgcgcttaagatcgcctgaccacggctcagacctttaccacgaccgcgaccatccagccaggcttgcagttcgtaccattccgcaggttcaataatggggccacagtccagttcaacaggacgcagggtaataggatcacgttggatacgatacccttcgattttggtcgttggggtcccatctggtttcttcttataaatgacttccgcagcaaatccggcgatgcggggatcgcgtaaaatgcgcatcactgtggcggggtcccacgccgagctcgcagttttcttgccgatggtttcaccacgcgttggaaccgcatccgcatccatacgtttgcacaagccggtaattgaaccaggatggatcgccgcctgggaacctggtttaaacggcagatgcttatgagttttaatttcgcgccaccaccaacgaatgacatctggttcaaattcaaacgggcctgtcagcggggttgttgaatgggccagtttattaatcaccacgttaaccatgcgaccattacgggtaatttctttggtttcgctaacaagctcgaagccgtaaggcgctttaccgcccacatatccacccagctcacgttgtaaattcttagtatccaggattttggcgctctttaaactgctctccttgtgtgaggcatccagacgcatgatcaggtgaatcaggtccatgacattgccttggcgaaacacaccctcttgggttgagacgatggtgacacctaacgccagtaactcgcttacgatggggattgcatccatcactttgagacgagaaaagcgagaaacatcgtacacgataatcatatttaagcggcccgcacgacactcatttaaaatacgctcaaattcaggacgttcggctgtaccaaaagcgctggtccctggagcctctgaaaagtgacccacaaaacgaaagcgaccaccatcacgttccacctcacgttgcagatctgcggctttatcctcattcgcggagcgttgggttgcgggggacgcggctgagctgttttcacgttcacgagattggcgatcgtaagcacccgcgtacgtgtccatattgttcctcctattggatctgagtacatgcaaccattatcaccgccagaggtaaaatagtcaacacgcgcggtgttagatatttatcccttgcggtgatattggtaacgaatcagacaattgacggcgacaccatcgaatggcgcaaacctttcgcggtatggcatgatagcgcccggaagagagtcaattcagggtggtgaatgtataagaaggagatatacatatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgactcgagtctggtccaccgctgagcaataactagcataaccccttggggcctctaaacgggtcttgaggggttttttgcgatctgaagcgaaccatgacgttagttcctattccgaagttcctattctctagaaagtataggaacttcgaagcagctcttgttcagaacgctcggtcttgcacaccgggcgttttttctttgtgagtccagcgtcgtggtttgtctggtcaaccaccgcggactcagtggtgtacggtacaaacccagtgccccaactggggtaacctaggagttctctcagttgggggcgtaggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatagtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggcggaaaaaccccgccctgtcaggggcggggtttttggcgtacgtctcgaggtcgacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataactgcaggaggtacccggggatcctctagaaataattttgtttaactttaagaaggagatatacatatgcggggttctcatcatcatcatcatcatggtatggctagcatgactggtggacagcaaatgggtcgggatctgtacgacgatgacgataaggatccgatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcctgcaggatatctggatccacgaagcttcccaccgctgagcaataactagcataaccccttggggcctctaaacgggtattgaggggttttttgccactcactacagcagagccatgtagggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggcccgctaacgcggcaccccatccccccaggggctgcgcccctcggccgcgaacgacctcaccccaaaaatggcagccacgtagaaagccagtccgcagaaacggtgctgaccccggatgaatgtcagctactgggctatctggacaagggaaaacgcaagcgtttagtttctcgtttcttcttcttccttcatagcttgatccgaaaagttactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatcccaggtcttcacttttgaaaagcattgacaattaatcatcggctcgtataatgtgtggaagtgcgggtgccagggcgtgcccaggggctccccgggcgcgtactccggccggcttgtcgacgacggcggactccgtcgtcaggatcatccgggctctaactaaaaacaccctaacgggtgttttttcttttctggtctcccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagacactgctctttaacaatttatcagatccaataggaggaacaatatgtctaccaaaaaaaaaccacttacacaggaacagttggaagatgcccggcgtcttaaggcgatttatgagaagaagaaaaacgaattagggctgtctcaagaatcagtggctgataagatgggtatgggacagtccggcgtcggggcgttatttaatggtataaacgcattaaatgcctacaacgccgctttactgaccaaaattttgaaagtctccgtagaagaatttagtccatctattgctagagagatatacgagatgtacgaagccgttagtatgcaaccatcgctgcgttcggagtatgaatatccagtcttttctcatgtccaagccggaatgttttcccccgagttgcggacatttacgaagggagacgcagagcgttgggtgtcgacaactaagaaagcctccgactctgccttctggctggaggtagaaggaaactctatgaccgcccctactggatctaagccttccttccctgatgggatgttgattctggttgatcctgaacaagcggttgagcccggggatttctgcattgcaagattaggcggcgacgaatttactttcaaaaaacttattcgcgattcgggacaagttttcctgcaaccactgaacccgcaatacccaatgatcccttgtaatgaatcatgcagtgttgttggtaaggtgatagcttcgcagtggccagaggagacctttgggtaatactagagaaaaaaaaaccccgcttcggcggggtttttttttgatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

SEQ ID NO: 24 - DNA, 11685 bp, synthetic construct
  source 1..11685: mol_type = other DNA
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaaacgcaaaaaaccccgcccctgacagggcggggttttttcgcgtgcgggtgccagggcgtgccctcgagttctctcagttgggggcgtgctattcgatagttgttaaggtcgcggtctgacgctcagtggaacaaccatctgcggtgataaattatctctggcggtgttgacataaataccactggcggtgatactgagcacatgtactcagatccaataggaggaacaatatgcgcgcgcttgttgtgattcgtttatcgcgtgtgacggacgccaccaccagtcccgaacgccaattagaaagctgtcaacaactttgtgctcaacgtggttgggatgtggtgggagtggccgaagaccttgatgtttcgggcgctgttgacccatttgatcgcaaacgtcgtcctaatttggcgcgttggctggcctttgaagaacagcctttcgatgtaattgtcgcatatcgcgttgaccgtctgacacgttccattcgccacctgcaacaacttgtgcattgggcggaagatcataagaaattagttgtttcggcaacagaggcccattttgacaccaccacacctttcgctgctgtagttattgccctgatgggtactgtcgctcaaatggagctcgaggcaattaaggaacgcaatcgctccgcggcccactttaacattcgtgctggcaagtatcgcggcagcttacccccgtggggttatctccccacacgtgtagatggcgaatggcgcctcgtccctgatccggtccaacgcgaacgtattctggaagtctaccatcgtgtagtggataatcatgaacccttacacctcgttgctcatgatctcaatcgccgcggcgttttgagccccaaagattattttgcccaattgcagggtcgtgaaccccaaggtcgcgaatggtccgccacagccttaaaacgctcaatgatttctgaagcaatgcttggctatgcaacgttgaatggcaaaactgttcgtgatgatgatggcgcgcccttggttcgcgccgaaccaattttgacacgcgaacaattagaagctcttcgtgcagaactggttaaaacatcgcgtgccaaaccggccgttagtactccttctcttctccttcgtgtcctgttttgtgctgtctgtggcgaacctgcctataaatttgctggtggcgggcgcaaacatcctcgttatcgttgtcgtagcatgggctttcccaaacattgtggcaatggtactgttgctatggcggaatgggatgccttttgtgaagaacaagtgttagaccttctgggcgatgcagaacgcctcgaaaaggtatgggtcgccgggagcgatagcgcagtggagctggctgaagtaaatgccgaactcgttgatttaactagcttgattggatctcctgcgtatcgcgccggatcgccacaacgtgaggccttagacgcgcgcatcgctgccttagctgcccgccaggaagaactcgaaggtttggaagcgcgtcctagcggatgggaatggcgtgaaactggtcaacgctttggagattggtggcgcgaacaagatacggctgccaagaatacatggctgcgttccatgaatgtgcgtcttacctttgatgtgcgtggtggtttaacccgtacaattgattttggcgacctgcaagaatatgaacaacacctgcgtctgggatcagtcgtggagcgcttgcatacaggcatgtcataaggcatctaactaaaaacaccctaacggcaaaataataaaaaagccggattaataatctggctttttatattctctcactaccatcggcgctacggcgtttcacttctgagttctgcagcggccgctactagtatggtaccattacgccgctacgtcttccgtgccgtcttgagcatcatcctcatcatcatccgttgggggtttagcccaagtaatactcgcacgtttctcaatcggggtgccttggccgcgtccggtcgttgatttggtcaccacaattttatcaacaaacaggcccacaaatacacgtttatcatccacacttgcacggccccaccagcttttcggacctgttgggtccgcatccgcatcttccggaaaccactgatccagcggcaatttgggtgcctcggccgcctcgagctctgccagacgttcctcggcgccctgttgacgtaaggttaatgccgcttgctgtttgcgaaaatgtttacgaccgaccggaccatcatatgccccggccgcacgatcctcatacagctcttcaagggcgttcagcgcatccgcacgttcggccaccagattggcgcgctctcctgatttctccggggcttcggtaagtttaccaaaacggcgcgccgcttcccataacaatgccagggtttcctcatcaccctctgcatgacgaattttattaaagatacgctcggctacaaatttatccagcgcggccatagacacattacaagttccctcatgttggcccggcgccgagggatcaacgactttgcgacggcgacaacgatatgaatctttaattgactcctcgccacgtttggatgtcataaccgcaccgcattcacaataaagtttatccatcgcgcttaagatcgcctgaccacggctcagacctttaccacgaccgcgaccatccagccaggcttgcagttcgtaccattccgcaggttcaataatggggccacagtccagttcaacaggacgcagggtaataggatcacgttggatacgatacccttcgattttggtcgttggggtcccatctggtttcttcttataaatgacttccgcagcaaatccggcgatgcggggatcgcgtaaaatgcgcatcactgtggcggggtcccacgccgagctcgcagttttcttgccgatggtttcaccacgcgttggaaccgcatccgcatccatacgtttgcacaagccggtaattgaaccaggatggatcgccgcctgggaacctggtttaaacggcagatgcttatgagttttaatttcgcgccaccaccaacgaatgacatctggttcaaattcaaacgggcctgtcagcggggttgttgaatgggccagtttattaatcaccacgttaaccatgcgaccattacgggtaatttctttggtttcgctaacaagctcgaagccgtaaggcgctttaccgcccacatatccacccagctcacgttgtaaattcttagtatccaggattttggcgctctttaaactgctctccttgtgtgaggcatccagacgcatgatcaggtgaatcaggtccatgacattgccttggcgaaacacaccctcttgggttgagacgatggtgacacctaacgccagtaactcgcttacgatggggattgcatccatcactttgagacgagaaaagcgagaaacatcgtacacgataatcatatttaagcggcccgcacgacactcatttaaaatacgctcaaattcaggacgttcggctgtaccaaaagcgctggtccctggagcctctgaaaagtgacccacaaaacgaaagcgaccaccatcacgttccacctcacgttgcagatctgcggctttatcctcattcgcggagcgttgggttgcgggggacgcggctgagctgttttcacgttcacgagattggcgatcgtaagcacccgcgtacgtgtccatattgttcctcctattggatctgagtacatgcaaccattatcaccgccagaggtaaaatagtcaacacgcgcggtgttagatatttatcccttgcggtgatattggtaacgaatcagacaattgacggcgacaccatcgaatggcgcaaacctttcgcggtatggcatgatagcgcccggaagagagtcaattcagggtggtgaatgtataagaaggagatatacatatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgactcgagtctggtccaccgctgagcaataactagcataaccccttggggcctctaaacgggtcttgaggggttttttgcgatctgaagcgaaccatgacgttagttcctattccgaagttcctattctctagaaagtataggaacttcgaagcagctcttgttcagaacgctcggtcttgcacaccgggcgttttttctttgtgagtccagcggccggcttgtcgacgacggcggtctccgtcgtcaggatcatccgggcatgccaacacaattaacatctcaatcaaggtaaatgctttttgctttttttgccaatcatcagataactatggcggcacgtgcattaaccacggttgtatcccgtctaaagtactcgtgtgcgggtgccagggcgtgcccttgggctccccgggcgcgtactccggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatagtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggcggaaaaaccccgccctgtcaggggcggggtttttggcgtacgtctcgaggtcgacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataactgcaggaggtacccggggatcctctagaaataattttgtttaactttaagaaggagatatacatatgcggggttctcatcatcatcatcatcatggtatggctagcatgactggtggacagcaaatgggtcgggatctgtacgacgatgacgataaggatccgatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcctgcaggatatctggatccacgaagcttcccaccgctgagcaataactagcataaccccttggggcctctaaacgggtattgaggggttttttgccactcactacagcagagccatgtagggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggcccgctaacgcggcaccccatccccccaggggctgcgcccctcggccgcgaacgacctcaccccaaaaatggcagccacgtagaaagccagtccgcagaaacggtgctgaccccggatgaatgtcagctactgggctatctggacaagggaaaacgcaagcgaaaagcattgacaattaatcatcggctcgtataatgtgtggaaagtgccccaactggggtaacctttgagttctctcagttgggggcgtttaataaactatggaagtatgtacagtcttgcaatgttgagtgaacaaacttccataataaaattgcgagtttttatttcgtttatttcaattaaggtaactaaaaaactcctttacgtcgtggtttgtctggtcaaccaccgcggtctcagtggtgtacggtacaaacccaattattgaagcggctaacgccgctttttttgtttctggtctccctttagtttctcgtttcttcttcttccttcatagcttgatccgaaaagttactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatcccaggtcttcacttttgaaaagcattgacaattaatcatcggctcgtataatgtgtggaagtgcgggtgccagggcgtgcccaggggctccccgggcgcgtactccggccggcttgtcgacgacggcggactccgtcgtcaggatcatccgggctctaactaaaaacaccctaacgggtgttttttcttttctggtctcccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagacactgctctttaacaatttatcagatccaataggaggaacaatatgtctaccaaaaaaaaaccacttacacaggaacagttggaagatgcccggcgtcttaaggcgatttatgagaagaagaaaaacgaattagggctgtctcaagaatcagtggctgataagatgggtatgggacagtccggcgtcggggcgttatttaatggtataaacgcattaaatgcctacaacgccgctttactgaccaaaattttgaaagtctccgtagaagaatttagtccatctattgctagagagatatacgagatgtacgaagccgttagtatgcaaccatcgctgcgttcggagtatgaatatccagtcttttctcatgtccaagccggaatgttttcccccgagttgcggacatttacgaagggagacgcagagcgttgggtgtcgacaactaagaaagcctccgactctgccttctggctggaggtagaaggaaactctatgaccgcccctactggatctaagccttccttccctgatgggatgttgattctggttgatcctgaacaagcggttgagcccggggatttctgcattgcaagattaggcggcgacgaatttactttcaaaaaacttattcgcgattcgggacaagttttcctgcaaccactgaacccgcaatacccaatgatcccttgtaatgaatcatgcagtgttgttggtaaggtgatagcttcgcagtggccagaggagacctttgggtaatactagagaaaaaaaaaccccgcttcggcggggtttttttttgatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

SEQ ID NO: 25 - DNA, 12328 bp, synthetic construct
  source 1..12328: mol_type = other DNA
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaaacgcaaaaaaccccgcccctgacagggcggggttttttcgcgtgcgggtgccagggcgtgccctcgagttctctcagttgggggcgtgctattcgatagttgttaaggtcgcggtctgacgctcagtggaacaaccatctgcggtgataaattatctctggcggtgttgacataaataccactggcggtgatactgagcacatgtactcagatccaataggaggaacaatatgcgcgcgcttgttgtgattcgtttatcgcgtgtgacggacgccaccaccagtcccgaacgccaattagaaagctgtcaacaactttgtgctcaacgtggttgggatgtggtgggagtggccgaagaccttgatgtttcgggcgctgttgacccatttgatcgcaaacgtcgtcctaatttggcgcgttggctggcctttgaagaacagcctttcgatgtaattgtcgcatatcgcgttgaccgtctgacacgttccattcgccacctgcaacaacttgtgcattgggcggaagatcataagaaattagttgtttcggcaacagaggcccattttgacaccaccacacctttcgctgctgtagttattgccctgatgggtactgtcgctcaaatggagctcgaggcaattaaggaacgcaatcgctccgcggcccactttaacattcgtgctggcaagtatcgcggcagcttacccccgtggggttatctccccacacgtgtagatggcgaatggcgcctcgtccctgatccggtccaacgcgaacgtattctggaagtctaccatcgtgtagtggataatcatgaacccttacacctcgttgctcatgatctcaatcgccgcggcgttttgagccccaaagattattttgcccaattgcagggtcgtgaaccccaaggtcgcgaatggtccgccacagccttaaaacgctcaatgatttctgaagcaatgcttggctatgcaacgttgaatggcaaaactgttcgtgatgatgatggcgcgcccttggttcgcgccgaaccaattttgacacgcgaacaattagaagctcttcgtgcagaactggttaaaacatcgcgtgccaaaccggccgttagtactccttctcttctccttcgtgtcctgttttgtgctgtctgtggcgaacctgcctataaatttgctggtggcgggcgcaaacatcctcgttatcgttgtcgtagcatgggctttcccaaacattgtggcaatggtactgttgctatggcggaatgggatgccttttgtgaagaacaagtgttagaccttctgggcgatgcagaacgcctcgaaaaggtatgggtcgccgggagcgatagcgcagtggagctggctgaagtaaatgccgaactcgttgatttaactagcttgattggatctcctgcgtatcgcgccggatcgccacaacgtgaggccttagacgcgcgcatcgctgccttagctgcccgccaggaagaactcgaaggtttggaagcgcgtcctagcggatgggaatggcgtgaaactggtcaacgctttggagattggtggcgcgaacaagatacggctgccaagaatacatggctgcgttccatgaatgtgcgtcttacctttgatgtgcgtggtggtttaacccgtacaattgattttggcgacctgcaagaatatgaacaacacctgcgtctgggatcagtcgtggagcgcttgcatacaggcatgtcataaggcatctaactaaaaacaccctaacggcaaaataataaaaaagccggattaataatctggctttttatattctctcactaccatcggcgctacggcgtttcacttctgagttctgcagcggccgctactagtatggtaccattacgccgctacgtcttccgtgccgtcttgagcatcatcctcatcatcatccgttgggggtttagcccaagtaatactcgcacgtttctcaatcggggtgccttggccgcgtccggtcgttgatttggtcaccacaattttatcaacaaacaggcccacaaatacacgtttatcatccacacttgcacggccccaccagcttttcggacctgttgggtccgcatccgcatcttccggaaaccactgatccagcggcaatttgggtgcctcggccgcctcgagctctgccagacgttcctcggcgccctgttgacgtaaggttaatgccgcttgctgtttgcgaaaatgtttacgaccgaccggaccatcatatgccccggccgcacgatcctcatacagctcttcaagggcgttcagcgcatccgcacgttcggccaccagattggcgcgctctcctgatttctccggggcttcggtaagtttaccaaaacggcgcgccgcttcccataacaatgccagggtttcctcatcaccctctgcatgacgaattttattaaagatacgctcggctacaaatttatccagcgcggccatagacacattacaagttccctcatgttggcccggcgccgagggatcaacgactttgcgacggcgacaacgatatgaatctttaattgactcctcgccacgtttggatgtcataaccgcaccgcattcacaataaagtttatccatcgcgcttaagatcgcctgaccacggctcagacctttaccacgaccgcgaccatccagccaggcttgcagttcgtaccattccgcaggttcaataatggggccacagtccagttcaacaggacgcagggtaataggatcacgttggatacgatacccttcgattttggtcgttggggtcccatctggtttcttcttataaatgacttccgcagcaaatccggcgatgcggggatcgcgtaaaatgcgcatcactgtggcggggtcccacgccgagctcgcagttttcttgccgatggtttcaccacgcgttggaaccgcatccgcatccatacgtttgcacaagccggtaattgaaccaggatggatcgccgcctgggaacctggtttaaacggcagatgcttatgagttttaatttcgcgccaccaccaacgaatgacatctggttcaaattcaaacgggcctgtcagcggggttgttgaatgggccagtttattaatcaccacgttaaccatgcgaccattacgggtaatttctttggtttcgctaacaagctcgaagccgtaaggcgctttaccgcccacatatccacccagctcacgttgtaaattcttagtatccaggattttggcgctctttaaactgctctccttgtgtgaggcatccagacgcatgatcaggtgaatcaggtccatgacattgccttggcgaaacacaccctcttgggttgagacgatggtgacacctaacgccagtaactcgcttacgatggggattgcatccatcactttgagacgagaaaagcgagaaacatcgtacacgataatcatatttaagcggcccgcacgacactcatttaaaatacgctcaaattcaggacgttcggctgtaccaaaagcgctggtccctggagcctctgaaaagtgacccacaaaacgaaagcgaccaccatcacgttccacctcacgttgcagatctgcggctttatcctcattcgcggagcgttgggttgcgggggacgcggctgagctgttttcacgttcacgagattggcgatcgtaagcacccgcgtacgtgtccatattgttcctcctattggatctgagtacatgcaaccattatcaccgccagaggtaaaatagtcaacacgcgcggtgttagatatttatcccttgcggtgatattggtaacgaatcagacaattgacggcgacaccatcgaatggcgcaaacctttcgcggtatggcatgatagcgcccggaagagagtcaattcagggtggtgaatgtataagaaggagatatacatatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgactcgagtctggtccaccgctgagcaataactagcataaccccttggggcctctaaacgggtcttgaggggttttttgcgatctgaagcgaaccatgacgttagttcctattccgaagttcctattctctagaaagtataggaacttcgaagcagctcttgttcagaacgctcggtcttgcacaccgggcgttttttctttgtgagtccagcggccggcttgtcgacgacggcggtctccgtcgtcaggatcatccgggcatgccaacacaattaacatctcaatcaaggtaaatgctttttgctttttttgccaatcatcagataactatggcggcacgtgcattaaccacggttgtatcccgtctaaagtactcgtgtgcgggtgccagggcgtgcccttgggctccccgggcgcgtactccggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatagtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggcggaaaaaccccgccctgtcaggggcggggtttttggcgtacgtctcgaggtcgacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataactgcaggaggtacccggggatcctctagaaataattttgtttaactttaagaaggagatatacatatgcggggttctcatcatcatcatcatcatggtatggctagcatgactggtggacagcaaatgggtcgggatctgtacgacgatgacgataaggatccgatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcctgcaggatatctggatccacgaagcttcccaccgctgagcaataactagcataaccccttggggcctctaaacgggtattgaggggttttttgccactcactacagcagagccatgtagggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggcccgctaacgcggcaccccatccccccaggggctgcgcccctcggccgcgaacgacctcaccccaaaaatggcagccacgtagaaagccagtccgcagaaacggtgctgaccccggatgaatgtcagctactgggctatctggacaagggaaaacgcaagcgaaaagcattgacaattaatcatcggctcgtataatgtgtggaaagtgccccaactggggtaacctttgagttctctcagttgggggcgtttaataaactatggaagtatgtacagtcttgcaatgttgagtgaacaaacttccataataaaattgcgagtttttatttcgtttatttcaattaaggtaactaaaaaactcctttacgtcgtggtttgtctggtcaaccaccgcggtctcagtggtgtacggtacaaacccaattattgaagcggctaacgccgctttttttgtttctggtctcccgtcgtggtttgtctggtcaaccaccgcggactcagtggtgtacggtacaaacccagtgccccaactggggtaacctaggagttctctcagttgggggcgtaacaattgggatccactgctctttaacaatttatcagatccaataggaggaacaatatgagagaccagtaactcggtaccaaattccagaaaagacacccgaaagggtgttttttcgttttggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaaagtatacatcatggcagacaaacaaaaggatggaatcaaagttaacttcaaaattagacacaacattgaagatggaagcgttcaactagcagaccattatcaacgaaatactccaattggcgatggtctcgtaacgcaaaaaaccccgcccctgacagggcggggttttttcgctttagtttctcgtttcttcttcttccttcatagcttgatccgaaaagttactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatcccaggtcttcacttttgaaaagcattgacaattaatcatcggctcgtataatgtgtggaagtgcgggtgccagggcgtgcccaggggctccccgggcgcgtactccggccggcttgtcgacgacggcggactccgtcgtcaggatcatccgggctctaactaaaaacaccctaacgggtgttttttcttttctggtctcccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagacactgctctttaacaatttatcagatccaataggaggaacaatatgtctaccaaaaaaaaaccacttacacaggaacagttggaagatgcccggcgtcttaaggcgatttatgagaagaagaaaaacgaattagggctgtctcaagaatcagtggctgataagatgggtatgggacagtccggcgtcggggcgttatttaatggtataaacgcattaaatgcctacaacgccgctttactgaccaaaattttgaaagtctccgtagaagaatttagtccatctattgctagagagatatacgagatgtacgaagccgttagtatgcaaccatcgctgcgttcggagtatgaatatccagtcttttctcatgtccaagccggaatgttttcccccgagttgcggacatttacgaagggagacgcagagcgttgggtgtcgacaactaagaaagcctccgactctgccttctggctggaggtagaaggaaactctatgaccgcccctactggatctaagccttccttccctgatgggatgttgattctggttgatcctgaacaagcggttgagcccggggatttctgcattgcaagattaggcggcgacgaatttactttcaaaaaacttattcgcgattcgggacaagttttcctgcaaccactgaacccgcaatacccaatgatcccttgtaatgaatcatgcagtgttgttggtaaggtgatagcttcgcagtggccagaggagacctttgggtaatactagagaaaaaaaaaccccgcttcggcggggtttttttttgatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

SEQ ID NO: 26 - DNA, 12331 bp, synthetic construct
  source 1..12331: mol_type = other DNA
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaaacgcaaaaaaccccgcccctgacagggcggggttttttcgcgtgcgggtgccagggcgtgccctcgagttctctcagttgggggcgtgctattcgatagttgttaaggtcgcggtctgacgctcagtggaacaaccatctgcggtgataaattatctctggcggtgttgacataaataccactggcggtgatactgagcacatgtactcagatccaataggaggaacaatatgcgcgcgcttgttgtgattcgtttatcgcgtgtgacggacgccaccaccagtcccgaacgccaattagaaagctgtcaacaactttgtgctcaacgtggttgggatgtggtgggagtggccgaagaccttgatgtttcgggcgctgttgacccatttgatcgcaaacgtcgtcctaatttggcgcgttggctggcctttgaagaacagcctttcgatgtaattgtcgcatatcgcgttgaccgtctgacacgttccattcgccacctgcaacaacttgtgcattgggcggaagatcataagaaattagttgtttcggcaacagaggcccattttgacaccaccacacctttcgctgctgtagttattgccctgatgggtactgtcgctcaaatggagctcgaggcaattaaggaacgcaatcgctccgcggcccactttaacattcgtgctggcaagtatcgcggcagcttacccccgtggggttatctccccacacgtgtagatggcgaatggcgcctcgtccctgatccggtccaacgcgaacgtattctggaagtctaccatcgtgtagtggataatcatgaacccttacacctcgttgctcatgatctcaatcgccgcggcgttttgagccccaaagattattttgcccaattgcagggtcgtgaaccccaaggtcgcgaatggtccgccacagccttaaaacgctcaatgatttctgaagcaatgcttggctatgcaacgttgaatggcaaaactgttcgtgatgatgatggcgcgcccttggttcgcgccgaaccaattttgacacgcgaacaattagaagctcttcgtgcagaactggttaaaacatcgcgtgccaaaccggccgttagtactccttctcttctccttcgtgtcctgttttgtgctgtctgtggcgaacctgcctataaatttgctggtggcgggcgcaaacatcctcgttatcgttgtcgtagcatgggctttcccaaacattgtggcaatggtactgttgctatggcggaatgggatgccttttgtgaagaacaagtgttagaccttctgggcgatgcagaacgcctcgaaaaggtatgggtcgccgggagcgatagcgcagtggagctggctgaagtaaatgccgaactcgttgatttaactagcttgattggatctcctgcgtatcgcgccggatcgccacaacgtgaggccttagacgcgcgcatcgctgccttagctgcccgccaggaagaactcgaaggtttggaagcgcgtcctagcggatgggaatggcgtgaaactggtcaacgctttggagattggtggcgcgaacaagatacggctgccaagaatacatggctgcgttccatgaatgtgcgtcttacctttgatgtgcgtggtggtttaacccgtacaattgattttggcgacctgcaagaatatgaacaacacctgcgtctgggatcagtcgtggagcgcttgcatacaggcatgtcataaggcatctaactaaaaacaccctaacggcaaaataataaaaaagccggattaataatctggctttttatattctctcactaccatcggcgctacggcgtttcacttctgagttctgcagcggccgctactagtatggtaccattacgccgctacgtcttccgtgccgtcttgagcatcatcctcatcatcatccgttgggggtttagcccaagtaatactcgcacgtttctcaatcggggtgccttggccgcgtccggtcgttgatttggtcaccacaattttatcaacaaacaggcccacaaatacacgtttatcatccacacttgcacggccccaccagcttttcggacctgttgggtccgcatccgcatcttccggaaaccactgatccagcggcaatttgggtgcctcggccgcctcgagctctgccagacgttcctcggcgccctgttgacgtaaggttaatgccgcttgctgtttgcgaaaatgtttacgaccgaccggaccatcatatgccccggccgcacgatcctcatacagctcttcaagggcgttcagcgcatccgcacgttcggccaccagattggcgcgctctcctgatttctccggggcttcggtaagtttaccaaaacggcgcgccgcttcccataacaatgccagggtttcctcatcaccctctgcatgacgaattttattaaagatacgctcggctacaaatttatccagcgcggccatagacacattacaagttccctcatgttggcccggcgccgagggatcaacgactttgcgacggcgacaacgatatgaatctttaattgactcctcgccacgtttggatgtcataaccgcaccgcattcacaataaagtttatccatcgcgcttaagatcgcctgaccacggctcagacctttaccacgaccgcgaccatccagccaggcttgcagttcgtaccattccgcaggttcaataatggggccacagtccagttcaacaggacgcagggtaataggatcacgttggatacgatacccttcgattttggtcgttggggtcccatctggtttcttcttataaatgacttccgcagcaaatccggcgatgcggggatcgcgtaaaatgcgcatcactgtggcggggtcccacgccgagctcgcagttttcttgccgatggtttcaccacgcgttggaaccgcatccgcatccatacgtttgcacaagccggtaattgaaccaggatggatcgccgcctgggaacctggtttaaacggcagatgcttatgagttttaatttcgcgccaccaccaacgaatgacatctggttcaaattcaaacgggcctgtcagcggggttgttgaatgggccagtttattaatcaccacgttaaccatgcgaccattacgggtaatttctttggtttcgctaacaagctcgaagccgtaaggcgctttaccgcccacatatccacccagctcacgttgtaaattcttagtatccaggattttggcgctctttaaactgctctccttgtgtgaggcatccagacgcatgatcaggtgaatcaggtccatgacattgccttggcgaaacacaccctcttgggttgagacgatggtgacacctaacgccagtaactcgcttacgatggggattgcatccatcactttgagacgagaaaagcgagaaacatcgtacacgataatcatatttaagcggcccgcacgacactcatttaaaatacgctcaaattcaggacgttcggctgtaccaaaagcgctggtccctggagcctctgaaaagtgacccacaaaacgaaagcgaccaccatcacgttccacctcacgttgcagatctgcggctttatcctcattcgcggagcgttgggttgcgggggacgcggctgagctgttttcacgttcacgagattggcgatcgtaagcacccgcgtacgtgtccatattgttcctcctattggatctgagtacatgcaaccattatcaccgccagaggtaaaatagtcaacacgcgcggtgttagatatttatcccttgcggtgatattggtaacgaatcagacaattgacggcgacaccatcgaatggcgcaaacctttcgcggtatggcatgatagcgcccggaagagagtcaattcagggtggtgaatgtataagaaggagatatacatatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgactcgagtctggtccaccgctgagcaataactagcataaccccttggggcctctaaacgggtcttgaggggttttttgcgatctgaagcgaaccatgacgttagttcctattccgaagttcctattctctagaaagtataggaacttcgaagcagctcttgttcagaacgctcggtcttgcacaccgggcgttttttctttgtgagtccagcgttagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccggagtacgcgcccggggagcccaagggcacgccctggcacccgcacacgagtactttagacgggatacaaccgtggttaatgcacgtgccgccatagttatctgatgattggcaaaaaaagcaaaaagcatttaccttgattgagatgttaattgtgttggcatgcccggatgatcctgacgacggagaccgccgtcgtcgacaagccggccgagcggaaaaaccccgccctgtcaggggcggggtttttggcgtacgtctcgaggtcgacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataactgcaggaggtacccggggatcctctagaaataattttgtttaactttaagaaggagatatacatatgcggggttctcatcatcatcatcatcatggtatggctagcatgactggtggacagcaaatgggtcgggatctgtacgacgatgacgataaggatccgatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcctgcaggatatctggatccacgaagcttcccaccgctgagcaataactagcataaccccttggggcctctaaacgggtattgaggggttttttgccactcactacagcagagccatgtagggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggcccgctaacgcggcaccccatccccccaggggctgcgcccctcggccgcgaacgacctcaccccaaaaatggcagccacgtagaaagccagtccgcagaaacggtgctgaccccggatgaatgtcagctactgggctatctggacaagggaaaacgcaagcgggagaccagaaacaaaaaaagcggcgttagccgcttcaataattgggtttgtaccgtacaccactgagaccgcggtggttgaccagacaaaccacgacgtaaaggagttttttagttaccttaattgaaataaacgaaataaaaactcgcaattttattatggaagtttgttcactcaacattgcaagactgtacatacttccatagtttattaaacgcccccaactgagagaactcaaaggttaccccagttggggcactttccacacattatacgagccgatgattaattgtcaagcctttttggtcgtggtttgtctggtcaaccaccgcggactcagtggtgtacggtacaaacccagtgccccaactggggtaacctaggagttctctcagttgggggcgtaacaattgggatccactgctctttaacaatttatcagatccaataggaggaacaatatgagagaccagtaactcggtaccaaattccagaaaagacacccgaaagggtgttttttcgttttggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaaagtatacatcatggcagacaaacaaaaggatggaatcaaagttaacttcaaaattagacacaacattgaagatggaagcgttcaactagcagaccattatcaacgaaatactccaattggcgatggtctcgtaacgcaaaaaaccccgcccctgacagggcggggttttttcgctttagtttctcgtttcttcttcttccttcatagcttgatccgaaaagttactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatcccaggtcttcacttttgaaaagcattgacaattaatcatcggctcgtataatgtgtggaagtgcgggtgccagggcgtgcccaggggctccccgggcgcgtactccggccggcttgtcgacgacggcggactccgtcgtcaggatcatccgggctctaactaaaaacaccctaacgggtgttttttcttttctggtctcccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagacactgctctttaacaatttatcagatccaataggaggaacaatatgtctaccaaaaaaaaaccacttacacaggaacagttggaagatgcccggcgtcttaaggcgatttatgagaagaagaaaaacgaattagggctgtctcaagaatcagtggctgataagatgggtatgggacagtccggcgtcggggcgttatttaatggtataaacgcattaaatgcctacaacgccgctttactgaccaaaattttgaaagtctccgtagaagaatttagtccatctattgctagagagatatacgagatgtacgaagccgttagtatgcaaccatcgctgcgttcggagtatgaatatccagtcttttctcatgtccaagccggaatgttttcccccgagttgcggacatttacgaagggagacgcagagcgttgggtgtcgacaactaagaaagcctccgactctgccttctggctggaggtagaaggaaactctatgaccgcccctactggatctaagccttccttccctgatgggatgttgattctggttgatcctgaacaagcggttgagcccggggatttctgcattgcaagattaggcggcgacgaatttactttcaaaaaacttattcgcgattcgggacaagttttcctgcaaccactgaacccgcaatacccaatgatcccttgtaatgaatcatgcagtgttgttggtaaggtgatagcttcgcagtggccagaggagacctttgggtaatactagagaaaaaaaaaccccgcttcggcggggtttttttttgatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

SEQ ID NO: 27 - DNA, 9493 bp, synthetic construct
  source 1..9493: mol_type = other DNA
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaaacgcaaaaaaccccgcccctgacagggcggggttttttcgcgtgcgggtgccagggcgtgccctcgagttctctcagttgggggcgtgctattcgatagttgttaaggtcgcggtctgacgctcagtggaacaaccatctgcggtgataaattatctctggcggtgttgacataaataccactggcggtgatactgagcacatgtactcagatccaataggaggaacaatatgcgcgcgcttgttgtgattcgtttatcgcgtgtgacggacgccaccaccagtcccgaacgccaattagaaagctgtcaacaactttgtgctcaacgtggttgggatgtggtgggagtggccgaagaccttgatgtttcgggcgctgttgacccatttgatcgcaaacgtcgtcctaatttggcgcgttggctggcctttgaagaacagcctttcgatgtaattgtcgcatatcgcgttgaccgtctgacacgttccattcgccacctgcaacaacttgtgcattgggcggaagatcataagaaattagttgtttcggcaacagaggcccattttgacaccaccacacctttcgctgctgtagttattgccctgatgggtactgtcgctcaaatggagctcgaggcaattaaggaacgcaatcgctccgcggcccactttaacattcgtgctggcaagtatcgcggcagcttacccccgtggggttatctccccacacgtgtagatggcgaatggcgcctcgtccctgatccggtccaacgcgaacgtattctggaagtctaccatcgtgtagtggataatcatgaacccttacacctcgttgctcatgatctcaatcgccgcggcgttttgagccccaaagattattttgcccaattgcagggtcgtgaaccccaaggtcgcgaatggtccgccacagccttaaaacgctcaatgatttctgaagcaatgcttggctatgcaacgttgaatggcaaaactgttcgtgatgatgatggcgcgcccttggttcgcgccgaaccaattttgacacgcgaacaattagaagctcttcgtgcagaactggttaaaacatcgcgtgccaaaccggccgttagtactccttctcttctccttcgtgtcctgttttgtgctgtctgtggcgaacctgcctataaatttgctggtggcgggcgcaaacatcctcgttatcgttgtcgtagcatgggctttcccaaacattgtggcaatggtactgttgctatggcggaatgggatgccttttgtgaagaacaagtgttagaccttctgggcgatgcagaacgcctcgaaaaggtatgggtcgccgggagcgatagcgcagtggagctggctgaagtaaatgccgaactcgttgatttaactagcttgattggatctcctgcgtatcgcgccggatcgccacaacgtgaggccttagacgcgcgcatcgctgccttagctgcccgccaggaagaactcgaaggtttggaagcgcgtcctagcggatgggaatggcgtgaaactggtcaacgctttggagattggtggcgcgaacaagatacggctgccaagaatacatggctgcgttccatgaatgtgcgtcttacctttgatgtgcgtggtggtttaacccgtacaattgattttggcgacctgcaagaatatgaacaacacctgcgtctgggatcagtcgtggagcgcttgcatacaggcatgtcataaggcatctaactaaaaacaccctaacggcaaaataataaaaaagccggattaataatctggctttttatattctctcactaccatcggcgctacggcgtttcacttctgagttctgcagcggccgctactagtatggtaccattacgccgctacgtcttccgtgccgtcttgagcatcatcctcatcatcatccgttgggggtttagcccaagtaatactcgcacgtttctcaatcggggtgccttggccgcgtccggtcgttgatttggtcaccacaattttatcaacaaacaggcccacaaatacacgtttatcatccacacttgcacggccccaccagcttttcggacctgttgggtccgcatccgcatcttccggaaaccactgatccagcggcaatttgggtgcctcggccgcctcgagctctgccagacgttcctcggcgccctgttgacgtaaggttaatgccgcttgctgtttgcgaaaatgtttacgaccgaccggaccatcatatgccccggccgcacgatcctcatacagctcttcaagggcgttcagcgcatccgcacgttcggccaccagattggcgcgctctcctgatttctccggggcttcggtaagtttaccaaaacggcgcgccgcttcccataacaatgccagggtttcctcatcaccctctgcatgacgaattttattaaagatacgctcggctacaaatttatccagcgcggccatagacacattacaagttccctcatgttggcccggcgccgagggatcaacgactttgcgacggcgacaacgatatgaatctttaattgactcctcgccacgtttggatgtcataaccgcaccgcattcacaataaagtttatccatcgcgcttaagatcgcctgaccacggctcagacctttaccacgaccgcgaccatccagccaggcttgcagttcgtaccattccgcaggttcaataatggggccacagtccagttcaacaggacgcagggtaataggatcacgttggatacgatacccttcgattttggtcgttggggtcccatctggtttcttcttataaatgacttccgcagcaaatccggcgatgcggggatcgcgtaaaatgcgcatcactgtggcggggtcccacgccgagctcgcagttttcttgccgatggtttcaccacgcgttggaaccgcatccgcatccatacgtttgcacaagccggtaattgaaccaggatggatcgccgcctgggaacctggtttaaacggcagatgcttatgagttttaatttcgcgccaccaccaacgaatgacatctggttcaaattcaaacgggcctgtcagcggggttgttgaatgggccagtttattaatcaccacgttaaccatgcgaccattacgggtaatttctttggtttcgctaacaagctcgaagccgtaaggcgctttaccgcccacatatccacccagctcacgttgtaaattcttagtatccaggattttggcgctctttaaactgctctccttgtgtgaggcatccagacgcatgatcaggtgaatcaggtccatgacattgccttggcgaaacacaccctcttgggttgagacgatggtgacacctaacgccagtaactcgcttacgatggggattgcatccatcactttgagacgagaaaagcgagaaacatcgtacacgataatcatatttaagcggcccgcacgacactcatttaaaatacgctcaaattcaggacgttcggctgtaccaaaagcgctggtccctggagcctctgaaaagtgacccacaaaacgaaagcgaccaccatcacgttccacctcacgttgcagatctgcggctttatcctcattcgcggagcgttgggttgcgggggacgcggctgagctgttttcacgttcacgagattggcgatcgtaagcacccgcgtacgtgtccatattgttcctcctattggatctgagtacatgcaaccattatcaccgccagaggtaaaatagtcaacacgcgcggtgttagatatttatcccttgcggtgatattggtaacgaatcagacaattgacggcgacaccatcgaatggcgcaaacctttcgcggtatggcatgatagcgcccggaagagagtcaattcagggtggtgaatgtataagaaggagatatacatatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgactcgagtctggtccaccgctgagcaataactagcataaccccttggggcctctaaacgggtcttgaggggttttttgcgatctgaagcgaaccatgacgttagttcctattccgaagttcctattctctagaaagtataggaacttcgaagcagctcttgttcagaacgctcggtcttgcacaccgggcgttttttctttgtgagtccagcgtcgtggtttgtctggtcaaccaccgcggactcagtggtgtacggtacaaacccagtgccccaactggggtaacctaggagttctctcagttgggggcgtaagcggaaaaaccccgccctgtcaggggcggggtttttggcgtacgtctcgaggtcgacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataactgcaggaggtacccggggatcctctagaaataattttgtttaactttaagaaggagatatacatatgcggggttctcatcatcatcatcatcatggtatggctagcatgactggtggacagcaaatgggtcgggatctgtacgacgatgacgataaggatccgatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcctgcaggatatctggatccacgaagcttcccaccgctgagcaataactagcataaccccttggggcctctaaacgggtattgaggggttttttgccactcactacagcagagcagccacgtagaaagccagtccgcagaaacggtgctgaccccggatgaatgtcagctactgggctatctggacaagggaaaacgcaagcgtttagtttctcgtttcttcttcttccttcatagcttgatccgaaaagttactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatcccaggtcttcacttttgaaaagcattgacaattaatcatcggctcgtataatgtgtggaagtgcgggtgccagggcgtgcccaggggctccccgggcgcgtactccggccggcttgtcgacgacggcggactccgtcgtcaggatcatccgggctctaactaaaaacaccctaacgggtgttttttcttttctggtctcccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagacactgctctttaacaatttatcagatccaataggaggaacaatatgtctaccaaaaaaaaaccacttacacaggaacagttggaagatgcccggcgtcttaaggcgatttatgagaagaagaaaaacgaattagggctgtctcaagaatcagtggctgataagatgggtatgggacagtccggcgtcggggcgttatttaatggtataaacgcattaaatgcctacaacgccgctttactgaccaaaattttgaaagtctccgtagaagaatttagtccatctattgctagagagatatacgagatgtacgaagccgttagtatgcaaccatcgctgcgttcggagtatgaatatccagtcttttctcatgtccaagccggaatgttttcccccgagttgcggacatttacgaagggagacgcagagcgttgggtgtcgacaactaagaaagcctccgactctgccttctggctggaggtagaaggaaactctatgaccgcccctactggatctaagccttccttccctgatgggatgttgattctggttgatcctgaacaagcggttgagcccggggatttctgcattgcaagattaggcggcgacgaatttactttcaaaaaacttattcgcgattcgggacaagttttcctgcaaccactgaacccgcaatacccaatgatcccttgtaatgaatcatgcagtgttgttggtaaggtgatagcttcgcagtggccagaggagacctttgggtaatactagagaaaaaaaaaccccgcttcggcggggtttttttttgatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

SEQ ID NO: 28 - DNA, 4960 bp, synthetic construct
  source 1..4960: mol_type = other DNA
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaatatcctccttagttcctattccgaagttcctattctctagaaagtataggaacttcggcgcgcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaggcgcgccatttaaatgaagttcctattccgaagttcctattctctagaaagtataggaacttcgaagcagctccagcgaaaaaccccgccctgtcaggggcggggtttttggcgtacgtctcgaggtcgacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataactgcaggaggtacccggggatcctctagaaataattttgtttaactttaagaaggagatatacatatgcggggttctcatcatcatcatcatcatggtatggctagcatgactggtggacagcaaatgggtcgggatctgtacgacgatgacgataaggatccgatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcctgcaggatatctggatccacgaagcttcccaccgctgagcaataactagcataaccccttggggcctctaaacgggtattgaggggttttttgccactcactacagcagagccatgtagggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggcccgctaacgcggcaccccatccccccaggggctgcgcccctcggccgcgaacgacctcaccccaaaaatggcagccacgtagaaagccagtccgcagaaacggtgctgaccccggatgaatgtcagctactgggctatctggacaagggaaaacgcaagcgagacctgggatcctgggtattaaaaagaagatctatttatttagagatctgttctattgtgatctcttattaggatcgcactgccctgtggataacaaggatccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctgacagagttatccacagtagatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

SEQ ID NO: 29 - DNA, 11263 bp, synthetic construct
  source 1..11263: mol_type = other DNA
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaaacgcaaaaaaccccgcccctgacagggcggggttttttcgcgtgcgggtgccagggcgtgccctcgagttctctcagttgggggcgtgctattcgatagttgttaaggtcgcggtctgacgctcagtggaacaaccatctgcggtgataaattatctctggcggtgttgacataaataccactggcggtgatactgagcacatgtactcagatccaataggaggaacaatatgcgcgcgcttgttgtgattcgtttatcgcgtgtgacggacgccaccaccagtcccgaacgccaattagaaagctgtcaacaactttgtgctcaacgtggttgggatgtggtgggagtggccgaagaccttgatgtttcgggcgctgttgacccatttgatcgcaaacgtcgtcctaatttggcgcgttggctggcctttgaagaacagcctttcgatgtaattgtcgcatatcgcgttgaccgtctgacacgttccattcgccacctgcaacaacttgtgcattgggcggaagatcataagaaattagttgtttcggcaacagaggcccattttgacaccaccacacctttcgctgctgtagttattgccctgatgggtactgtcgctcaaatggagctcgaggcaattaaggaacgcaatcgctccgcggcccactttaacattcgtgctggcaagtatcgcggcagcttacccccgtggggttatctccccacacgtgtagatggcgaatggcgcctcgtccctgatccggtccaacgcgaacgtattctggaagtctaccatcgtgtagtggataatcatgaacccttacacctcgttgctcatgatctcaatcgccgcggcgttttgagccccaaagattattttgcccaattgcagggtcgtgaaccccaaggtcgcgaatggtccgccacagccttaaaacgctcaatgatttctgaagcaatgcttggctatgcaacgttgaatggcaaaactgttcgtgatgatgatggcgcgcccttggttcgcgccgaaccaattttgacacgcgaacaattagaagctcttcgtgcagaactggttaaaacatcgcgtgccaaaccggccgttagtactccttctcttctccttcgtgtcctgttttgtgctgtctgtggcgaacctgcctataaatttgctggtggcgggcgcaaacatcctcgttatcgttgtcgtagcatgggctttcccaaacattgtggcaatggtactgttgctatggcggaatgggatgccttttgtgaagaacaagtgttagaccttctgggcgatgcagaacgcctcgaaaaggtatgggtcgccgggagcgatagcgcagtggagctggctgaagtaaatgccgaactcgttgatttaactagcttgattggatctcctgcgtatcgcgccggatcgccacaacgtgaggccttagacgcgcgcatcgctgccttagctgcccgccaggaagaactcgaaggtttggaagcgcgtcctagcggatgggaatggcgtgaaactggtcaacgctttggagattggtggcgcgaacaagatacggctgccaagaatacatggctgcgttccatgaatgtgcgtcttacctttgatgtgcgtggtggtttaacccgtacaattgattttggcgacctgcaagaatatgaacaacacctgcgtctgggatcagtcgtggagcgcttgcatacaggcatgtcataaggcatctaactaaaaacaccctaacggcaaaataataaaaaagccggattaataatctggctttttatattctctcactaccatcggcgctacggcgtttcacttctgagttctgcagcggccgctactagtatggtaccattacgccgctacgtcttccgtgccgtcttgagcatcatcctcatcatcatccgttgggggtttagcccaagtaatactcgcacgtttctcaatcggggtgccttggccgcgtccggtcgttgatttggtcaccacaattttatcaacaaacaggcccacaaatacacgtttatcatccacacttgcacggccccaccagcttttcggacctgttgggtccgcatccgcatcttccggaaaccactgatccagcggcaatttgggtgcctcggccgcctcgagctctgccagacgttcctcggcgccctgttgacgtaaggttaatgccgcttgctgtttgcgaaaatgtttacgaccgaccggaccatcatatgccccggccgcacgatcctcatacagctcttcaagggcgttcagcgcatccgcacgttcggccaccagattggcgcgctctcctgatttctccggggcttcggtaagtttaccaaaacggcgcgccgcttcccataacaatgccagggtttcctcatcaccctctgcatgacgaattttattaaagatacgctcggctacaaatttatccagcgcggccatagacacattacaagttccctcatgttggcccggcgccgagggatcaacgactttgcgacggcgacaacgatatgaatctttaattgactcctcgccacgtttggatgtcataaccgcaccgcattcacaataaagtttatccatcgcgcttaagatcgcctgaccacggctcagacctttaccacgaccgcgaccatccagccaggcttgcagttcgtaccattccgcaggttcaataatggggccacagtccagttcaacaggacgcagggtaataggatcacgttggatacgatacccttcgattttggtcgttggggtcccatctggtttcttcttataaatgacttccgcagcaaatccggcgatgcggggatcgcgtaaaatgcgcatcactgtggcggggtcccacgccgagctcgcagttttcttgccgatggtttcaccacgcgttggaaccgcatccgcatccatacgtttgcacaagccggtaattgaaccaggatggatcgccgcctgggaacctggtttaaacggcagatgcttatgagttttaatttcgcgccaccaccaacgaatgacatctggttcaaattcaaacgggcctgtcagcggggttgttgaatgggccagtttattaatcaccacgttaaccatgcgaccattacgggtaatttctttggtttcgctaacaagctcgaagccgtaaggcgctttaccgcccacatatccacccagctcacgttgtaaattcttagtatccaggattttggcgctctttaaactgctctccttgtgtgaggcatccagacgcatgatcaggtgaatcaggtccatgacattgccttggcgaaacacaccctcttgggttgagacgatggtgacacctaacgccagtaactcgcttacgatggggattgcatccatcactttgagacgagaaaagcgagaaacatcgtacacgataatcatatttaagcggcccgcacgacactcatttaaaatacgctcaaattcaggacgttcggctgtaccaaaagcgctggtccctggagcctctgaaaagtgacccacaaaacgaaagcgaccaccatcacgttccacctcacgttgcagatctgcggctttatcctcattcgcggagcgttgggttgcgggggacgcggctgagctgttttcacgttcacgagattggcgatcgtaagcacccgcgtacgtgtccatattgttcctcctattggatctgagtacatgcaaccattatcaccgccagaggtaaaatagtcaacacgcgcggtgttagatatttatcccttgcggtgatattggtaacgaatcagacaattgacggcgacaccatcgaatggcgcaaacctttcgcggtatggcatgatagcgcccggaagagagtcaattcagggtggtgaatgtataagaaggagatatacatatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgactcgagtctggtccaccgctgagcaataactagcataaccccttggggcctctaaacgggtcttgaggggttttttgcgatctgaagcgaaccatgacgttagttcctattccgaagttcctattctctagaaagtataggaacttcgaagcagctcttgttcagaacgctcggtcttgcacaccgggcgttttttctttgtgagtccagcgtcgtggtttgtctggtcaaccaccgcggactcagtggtgtacggtacaaacccagtgccccaactggggtaacctaggagttctctcagttgggggcgtaggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggcggaaaaaccccgccctgtcaggggcggggtttttggcgtacgtctcgaggtcgacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataactgcaggaggtacccggggatcctctagaaataattttgtttaactttaagaaggagatatacatatggatattcagatgacccagagcccgtccagcctgtcagcaagcgttggcgaccgtgttacgattacctgccgtgcctcgcaagatgtgaacaccgcggtggcctggtatcagcaaaaaccgggcaaagcaccgaaactgctgatttatagtgcttcctttctgtactctggtgttccgtcccgtttctcaggctcgcgcagcggtaccgattttaccctgacgatcagctctctgcagccggaagacttcgccacgtattactgccagcaacactacacgaccccgccgaccttcggccagggcaccaaagtggaaatcaaaggcgccagcggcggtggcggttctggtggcggtggcagcggtggcggtggctcgagcgaagtccaactggtcgaatcgggtggtggtctggttcagccgggcggttcactgcgtctgtcgtgtgctgcctcgggtttcaacatcaaagatacctatattcattgggtgcgtcaggccccgggtaaaggtctggagtgggttgcccgcatctacccgaccaacggctatacgcgttacgccgatagcgttaaaggtcgctttaccatttctgcagacaccagtaaaaacacggcttatctgcagatgaactctctgcgtgcggaagacacggccgtgtattactgcagtcgctggggcggtgatggcttttatgcaatggactactggggccagggtaccctggttacggtcagctctggatcgcatcaccatcaccatcactaataggaattcctgcaggatatctggatccacgaagcttcccaccgctgagcaataactagcataaccccttggggcctctaaacgggtattgaggggttttttgccactcactacagcagagccatgtagggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggcccgctaacgcggcaccccatccccccaggggctgcgcccctcggccgcgaacgacctcaccccaaaaatggcagccacgtagaaagccagtccgcagaaacggtgctgaccccggatgaatgtcagctactgggctatctggacaagggaaaacgcaagcgtttagtttctcgtttcttcttcttccttcatagcttgatccgaaaagttactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatcccaggtcttcacttttgaaaagcattgacaattaatcatcggctcgtataatgtgtggaagtgcgggtgccagggcgtgcccaggggctccccgggcgcgtactccggccggcttgtcgacgacggcggactccgtcgtcaggatcatccgggctctaactaaaaacaccctaacgggtgttttttcttttctggtctcccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagacactgctctttaacaatttatcagatccaataggaggaacaatatgtctaccaaaaaaaaaccacttacacaggaacagttggaagatgcccggcgtcttaaggcgatttatgagaagaagaaaaacgaattagggctgtctcaagaatcagtggctgataagatgggtatgggacagtccggcgtcggggcgttatttaatggtataaacgcattaaatgcctacaacgccgctttactgaccaaaattttgaaagtctccgtagaagaatttagtccatctattgctagagagatatacgagatgtacgaagccgttagtatgcaaccatcgctgcgttcggagtatgaatatccagtcttttctcatgtccaagccggaatgttttcccccgagttgcggacatttacgaagggagacgcagagcgttgggtgtcgacaactaagaaagcctccgactctgccttctggctggaggtagaaggaaactctatgaccgcccctactggatctaagccttccttccctgatgggatgttgattctggttgatcctgaacaagcggttgagcccggggatttctgcattgcaagattaggcggcgacgaatttactttcaaaaaacttattcgcgattcgggacaagttttcctgcaaccactgaacccgcaatacccaatgatcccttgtaatgaatcatgcagtgttgttggtaaggtgatagcttcgcagtggccagaggagacctttgggtaatactagagaaaaaaaaaccccgcttcggcggggtttttttttgatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

SEQ ID NO: 30 - DNA, 11026 bp, synthetic construct
  source 1..11026: mol_type = other DNA
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaaacgcaaaaaaccccgcccctgacagggcggggttttttcgcgtgcgggtgccagggcgtgccctcgagttctctcagttgggggcgtgctattcgatagttgttaaggtcgcggtctgacgctcagtggaacaaccatctgcggtgataaattatctctggcggtgttgacataaataccactggcggtgatactgagcacatgtactcagatccaataggaggaacaatatgcgcgcgcttgttgtgattcgtttatcgcgtgtgacggacgccaccaccagtcccgaacgccaattagaaagctgtcaacaactttgtgctcaacgtggttgggatgtggtgggagtggccgaagaccttgatgtttcgggcgctgttgacccatttgatcgcaaacgtcgtcctaatttggcgcgttggctggcctttgaagaacagcctttcgatgtaattgtcgcatatcgcgttgaccgtctgacacgttccattcgccacctgcaacaacttgtgcattgggcggaagatcataagaaattagttgtttcggcaacagaggcccattttgacaccaccacacctttcgctgctgtagttattgccctgatgggtactgtcgctcaaatggagctcgaggcaattaaggaacgcaatcgctccgcggcccactttaacattcgtgctggcaagtatcgcggcagcttacccccgtggggttatctccccacacgtgtagatggcgaatggcgcctcgtccctgatccggtccaacgcgaacgtattctggaagtctaccatcgtgtagtggataatcatgaacccttacacctcgttgctcatgatctcaatcgccgcggcgttttgagccccaaagattattttgcccaattgcagggtcgtgaaccccaaggtcgcgaatggtccgccacagccttaaaacgctcaatgatttctgaagcaatgcttggctatgcaacgttgaatggcaaaactgttcgtgatgatgatggcgcgcccttggttcgcgccgaaccaattttgacacgcgaacaattagaagctcttcgtgcagaactggttaaaacatcgcgtgccaaaccggccgttagtactccttctcttctccttcgtgtcctgttttgtgctgtctgtggcgaacctgcctataaatttgctggtggcgggcgcaaacatcctcgttatcgttgtcgtagcatgggctttcccaaacattgtggcaatggtactgttgctatggcggaatgggatgccttttgtgaagaacaagtgttagaccttctgggcgatgcagaacgcctcgaaaaggtatgggtcgccgggagcgatagcgcagtggagctggctgaagtaaatgccgaactcgttgatttaactagcttgattggatctcctgcgtatcgcgccggatcgccacaacgtgaggccttagacgcgcgcatcgctgccttagctgcccgccaggaagaactcgaaggtttggaagcgcgtcctagcggatgggaatggcgtgaaactggtcaacgctttggagattggtggcgcgaacaagatacggctgccaagaatacatggctgcgttccatgaatgtgcgtcttacctttgatgtgcgtggtggtttaacccgtacaattgattttggcgacctgcaagaatatgaacaacacctgcgtctgggatcagtcgtggagcgcttgcatacaggcatgtcataaggcatctaactaaaaacaccctaacggcaaaataataaaaaagccggattaataatctggctttttatattctctcactaccatcggcgctacggcgtttcacttctgagttctgcagcggccgctactagtatggtaccattacgccgctacgtcttccgtgccgtcttgagcatcatcctcatcatcatccgttgggggtttagcccaagtaatactcgcacgtttctcaatcggggtgccttggccgcgtccggtcgttgatttggtcaccacaattttatcaacaaacaggcccacaaatacacgtttatcatccacacttgcacggccccaccagcttttcggacctgttgggtccgcatccgcatcttccggaaaccactgatccagcggcaatttgggtgcctcggccgcctcgagctctgccagacgttcctcggcgccctgttgacgtaaggttaatgccgcttgctgtttgcgaaaatgtttacgaccgaccggaccatcatatgccccggccgcacgatcctcatacagctcttcaagggcgttcagcgcatccgcacgttcggccaccagattggcgcgctctcctgatttctccggggcttcggtaagtttaccaaaacggcgcgccgcttcccataacaatgccagggtttcctcatcaccctctgcatgacgaattttattaaagatacgctcggctacaaatttatccagcgcggccatagacacattacaagttccctcatgttggcccggcgccgagggatcaacgactttgcgacggcgacaacgatatgaatctttaattgactcctcgccacgtttggatgtcataaccgcaccgcattcacaataaagtttatccatcgcgcttaagatcgcctgaccacggctcagacctttaccacgaccgcgaccatccagccaggcttgcagttcgtaccattccgcaggttcaataatggggccacagtccagttcaacaggacgcagggtaataggatcacgttggatacgatacccttcgattttggtcgttggggtcccatctggtttcttcttataaatgacttccgcagcaaatccggcgatgcggggatcgcgtaaaatgcgcatcactgtggcggggtcccacgccgagctcgcagttttcttgccgatggtttcaccacgcgttggaaccgcatccgcatccatacgtttgcacaagccggtaattgaaccaggatggatcgccgcctgggaacctggtttaaacggcagatgcttatgagttttaatttcgcgccaccaccaacgaatgacatctggttcaaattcaaacgggcctgtcagcggggttgttgaatgggccagtttattaatcaccacgttaaccatgcgaccattacgggtaatttctttggtttcgctaacaagctcgaagccgtaaggcgctttaccgcccacatatccacccagctcacgttgtaaattcttagtatccaggattttggcgctctttaaactgctctccttgtgtgaggcatccagacgcatgatcaggtgaatcaggtccatgacattgccttggcgaaacacaccctcttgggttgagacgatggtgacacctaacgccagtaactcgcttacgatggggattgcatccatcactttgagacgagaaaagcgagaaacatcgtacacgataatcatatttaagcggcccgcacgacactcatttaaaatacgctcaaattcaggacgttcggctgtaccaaaagcgctggtccctggagcctctgaaaagtgacccacaaaacgaaagcgaccaccatcacgttccacctcacgttgcagatctgcggctttatcctcattcgcggagcgttgggttgcgggggacgcggctgagctgttttcacgttcacgagattggcgatcgtaagcacccgcgtacgtgtccatattgttcctcctattggatctgagtacatgcaaccattatcaccgccagaggtaaaatagtcaacacgcgcggtgttagatatttatcccttgcggtgatattggtaacgaatcagacaattgacggcgacaccatcgaatggcgcaaacctttcgcggtatggcatgatagcgcccggaagagagtcaattcagggtggtgaatgtataagaaggagatatacatatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgactcgagtctggtccaccgctgagcaataactagcataaccccttggggcctctaaacgggtcttgaggggttttttgcgatctgaagcgaaccatgacgttagttcctattccgaagttcctattctctagaaagtataggaacttcgaagcagctcttgttcagaacgctcggtcttgcacaccgggcgttttttctttgtgagtccagcgtcgtggtttgtctggtcaaccaccgcggactcagtggtgtacggtacaaacccagtgccccaactggggtaacctaggagttctctcagttgggggcgtaggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggcggaaaaaccccgccctgtcaggggcggggtttttggcgtacgtctcgaggtcgacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataactgcaggaggtacccggggatcctctagaaataattttgtttaactttaagaaggagatatacatatgggacaccaccaccatcaccatcaccaccatcactcttcaggtcatattgatgatgacgacaaggcagccggctcaatcactaccttgcccgccttgccagaggatggtggatctggggcttttcctccgggtcattttaaggaccctaaacgcttgtactgtaaaaacggaggtttcttcttacgtattcatcccgatggacgtgttgacggcgtgcgcgaaaaatcagacccccacattaaattgcaactgcaagcagaagagcgtggcgtagtgagcattaagggagtttgtgcaaatcgctatttagccatgaaggaagatggacgtttgttggcctcgaagtgtgtgacagatgaatgcttcttttttgaacgcctggagtctaataactacaacacataccgttcgcgcaagtacacctcttggtacgttgcgttgaagcgtacaggacagtacaaacttggatctaaaacgggcccaggacagaaagcaatcctgttcttaccaatgtccgcgaaatcctaataggaattcctgcaggatatctggatccacgaagcttcccaccgctgagcaataactagcataaccccttggggcctctaaacgggtattgaggggttttttgccactcactacagcagagccatgtagggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggcccgctaacgcggcaccccatccccccaggggctgcgcccctcggccgcgaacgacctcaccccaaaaatggcagccacgtagaaagccagtccgcagaaacggtgctgaccccggatgaatgtcagctactgggctatctggacaagggaaaacgcaagcgtttagtttctcgtttcttcttcttccttcatagcttgatccgaaaagttactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatcccaggtcttcacttttgaaaagcattgacaattaatcatcggctcgtataatgtgtggaagtgcgggtgccagggcgtgcccaggggctccccgggcgcgtactccggccggcttgtcgacgacggcggactccgtcgtcaggatcatccgggctctaactaaaaacaccctaacgggtgttttttcttttctggtctcccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagacactgctctttaacaatttatcagatccaataggaggaacaatatgtctaccaaaaaaaaaccacttacacaggaacagttggaagatgcccggcgtcttaaggcgatttatgagaagaagaaaaacgaattagggctgtctcaagaatcagtggctgataagatgggtatgggacagtccggcgtcggggcgttatttaatggtataaacgcattaaatgcctacaacgccgctttactgaccaaaattttgaaagtctccgtagaagaatttagtccatctattgctagagagatatacgagatgtacgaagccgttagtatgcaaccatcgctgcgttcggagtatgaatatccagtcttttctcatgtccaagccggaatgttttcccccgagttgcggacatttacgaagggagacgcagagcgttgggtgtcgacaactaagaaagcctccgactctgccttctggctggaggtagaaggaaactctatgaccgcccctactggatctaagccttccttccctgatgggatgttgattctggttgatcctgaacaagcggttgagcccggggatttctgcattgcaagattaggcggcgacgaatttactttcaaaaaacttattcgcgattcgggacaagttttcctgcaaccactgaacccgcaatacccaatgatcccttgtaatgaatcatgcagtgttgttggtaaggtgatagcttcgcagtggccagaggagacctttgggtaatactagagaaaaaaaaaccccgcttcggcggggtttttttttgatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

SEQ ID NO: 31 - DNA, 10123 bp, synthetic construct
  source 1..10123: mol_type = other DNA
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaaacgcaaaaaaccccgcccctgacagggcggggttttttcgcgtgcgggtgccagggcgtgccctcgagttctctcagttgggggcgtgctattcgatagttgttaaggtcgcggtctgacgctcagtggaacaaccatctgcggtgataaattatctctggcggtgttgacataaataccactggcggtgatactgagcacatgtactcagatccaataggaggaacaatatgcgcgcgcttgttgtgattcgtttatcgcgtgtgacggacgccaccaccagtcccgaacgccaattagaaagctgtcaacaactttgtgctcaacgtggttgggatgtggtgggagtggccgaagaccttgatgtttcgggcgctgttgacccatttgatcgcaaacgtcgtcctaatttggcgcgttggctggcctttgaagaacagcctttcgatgtaattgtcgcatatcgcgttgaccgtctgacacgttccattcgccacctgcaacaacttgtgcattgggcggaagatcataagaaattagttgtttcggcaacagaggcccattttgacaccaccacacctttcgctgctgtagttattgccctgatgggtactgtcgctcaaatggagctcgaggcaattaaggaacgcaatcgctccgcggcccactttaacattcgtgctggcaagtatcgcggcagcttacccccgtggggttatctccccacacgtgtagatggcgaatggcgcctcgtccctgatccggtccaacgcgaacgtattctggaagtctaccatcgtgtagtggataatcatgaacccttacacctcgttgctcatgatctcaatcgccgcggcgttttgagccccaaagattattttgcccaattgcagggtcgtgaaccccaaggtcgcgaatggtccgccacagccttaaaacgctcaatgatttctgaagcaatgcttggctatgcaacgttgaatggcaaaactgttcgtgatgatgatggcgcgcccttggttcgcgccgaaccaattttgacacgcgaacaattagaagctcttcgtgcagaactggttaaaacatcgcgtgccaaaccggccgttagtactccttctcttctccttcgtgtcctgttttgtgctgtctgtggcgaacctgcctataaatttgctggtggcgggcgcaaacatcctcgttatcgttgtcgtagcatgggctttcccaaacattgtggcaatggtactgttgctatggcggaatgggatgccttttgtgaagaacaagtgttagaccttctgggcgatgcagaacgcctcgaaaaggtatgggtcgccgggagcgatagcgcagtggagctggctgaagtaaatgccgaactcgttgatttaactagcttgattggatctcctgcgtatcgcgccggatcgccacaacgtgaggccttagacgcgcgcatcgctgccttagctgcccgccaggaagaactcgaaggtttggaagcgcgtcctagcggatgggaatggcgtgaaactggtcaacgctttggagattggtggcgcgaacaagatacggctgccaagaatacatggctgcgttccatgaatgtgcgtcttacctttgatgtgcgtggtggtttaacccgtacaattgattttggcgacctgcaagaatatgaacaacacctgcgtctgggatcagtcgtggagcgcttgcatacaggcatgtcataaggcatctaactaaaaacaccctaacggcaaaataataaaaaagccggattaataatctggctttttatattctctcactaccatcggcgctacggcgtttcacttctgagttctgcagcggccgctactagtatggtaccattacgccgctacgtcttccgtgccgtcttgagcatcatcctcatcatcatccgttgggggtttagcccaagtaatactcgcacgtttctcaatcggggtgccttggccgcgtccggtcgttgatttggtcaccacaattttatcaacaaacaggcccacaaatacacgtttatcatccacacttgcacggccccaccagcttttcggacctgttgggtccgcatccgcatcttccggaaaccactgatccagcggcaatttgggtgcctcggccgcctcgagctctgccagacgttcctcggcgccctgttgacgtaaggttaatgccgcttgctgtttgcgaaaatgtttacgaccgaccggaccatcatatgccccggccgcacgatcctcatacagctcttcaagggcgttcagcgcatccgcacgttcggccaccagattggcgcgctctcctgatttctccggggcttcggtaagtttaccaaaacggcgcgccgcttcccataacaatgccagggtttcctcatcaccctctgcatgacgaattttattaaagatacgctcggctacaaatttatccagcgcggccatagacacattacaagttccctcatgttggcccggcgccgagggatcaacgactttgcgacggcgacaacgatatgaatctttaattgactcctcgccacgtttggatgtcataaccgcaccgcattcacaataaagtttatccatcgcgcttaagatcgcctgaccacggctcagacctttaccacgaccgcgaccatccagccaggcttgcagttcgtaccattccgcaggttcaataatggggccacagtccagttcaacaggacgcagggtaataggatcacgttggatacgatacccttcgattttggtcgttggggtcccatctggtttcttcttataaatgacttccgcagcaaatccggcgatgcggggatcgcgtaaaatgcgcatcactgtggcggggtcccacgccgagctcgcagttttcttgccgatggtttcaccacgcgttggaaccgcatccgcatccatacgtttgcacaagccggtaattgaaccaggatggatcgccgcctgggaacctggtttaaacggcagatgcttatgagttttaatttcgcgccaccaccaacgaatgacatctggttcaaattcaaacgggcctgtcagcggggttgttgaatgggccagtttattaatcaccacgttaaccatgcgaccattacgggtaatttctttggtttcgctaacaagctcgaagccgtaaggcgctttaccgcccacatatccacccagctcacgttgtaaattcttagtatccaggattttggcgctctttaaactgctctccttgtgtgaggcatccagacgcatgatcaggtgaatcaggtccatgacattgccttggcgaaacacaccctcttgggttgagacgatggtgacacctaacgccagtaactcgcttacgatggggattgcatccatcactttgagacgagaaaagcgagaaacatcgtacacgataatcatatttaagcggcccgcacgacactcatttaaaatacgctcaaattcaggacgttcggctgtaccaaaagcgctggtccctggagcctctgaaaagtgacccacaaaacgaaagcgaccaccatcacgttccacctcacgttgcagatctgcggctttatcctcattcgcggagcgttgggttgcgggggacgcggctgagctgttttcacgttcacgagattggcgatcgtaagcacccgcgtacgtgtccatattgttcctcctattggatctgagtacatgcaaccattatcaccgccagaggtaaaatagtcaacacgcgcggtgttagatatttatcccttgcggtgatattggtaacgaatcagacaattgacggcgacaccatcgaatggcgcaaacctttcgcggtatggcatgatagcgcccggaagagagtcaattcagggtggtgaatgtataagaaggagatatacatatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgactcgagtctggtccaccgctgagcaataactagcataaccccttggggcctctaaacgggtcttgaggggttttttgcgatctgaagcgaaccatgacgttagttcctattccgaagttcctattctctagaaagtataggaacttcgaagcagctcttgttcagaacgctcggtcttgcacaccgggcgttttttctttgtgagtccagcgtcgtggtttgtctggtcaaccaccgcggactcagtggtgtacggtacaaacccagtgccccaactggggtaacctaggagttctctcagttgggggcgtagcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgaaaaaccccgccctgtcaggggcggggtttttggcgtacgtctcgaggtcgacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataactgcaggaggtacccggggatcctctagaaataattttgtttaactttaagaaggagatatacatatgcggggttctcatcatcatcatcatcatggtatggctagcatgactggtggacagcaaatgggtcgggatctgtacgacgatgacgataaggatccgatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcctgcaggatatctggatccacgaagcttcccaccgctgagcaataactagcataaccccttggggcctctaaacgggtattgaggggttttttgccactcactacagcagagcagccacgtagaaagccagtccgcagaaacggtgctgaccccggatgaatgtcagctactgggctatctggacaagggaaaacgcaagcgtttagtttctcgtttcttcttcttccttcatagcttgatccgaaaagttactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatcccaggtcttcacttttgaaaagcattgacaattaatcatcggctcgtataatgtgtggaagtgcgggtgccagggcgtgcccaggggctccccgggcgcgtactccggccggcttgtcgacgacggcggactccgtcgtcaggatcatccgggctctaactaaaaacaccctaacgggtgttttttcttttctggtctcccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagacactgctctttaacaatttatcagatccaataggaggaacaatatgtctaccaaaaaaaaaccacttacacaggaacagttggaagatgcccggcgtcttaaggcgatttatgagaagaagaaaaacgaattagggctgtctcaagaatcagtggctgataagatgggtatgggacagtccggcgtcggggcgttatttaatggtataaacgcattaaatgcctacaacgccgctttactgaccaaaattttgaaagtctccgtagaagaatttagtccatctattgctagagagatatacgagatgtacgaagccgttagtatgcaaccatcgctgcgttcggagtatgaatatccagtcttttctcatgtccaagccggaatgttttcccccgagttgcggacatttacgaagggagacgcagagcgttgggtgtcgacaactaagaaagcctccgactctgccttctggctggaggtagaaggaaactctatgaccgcccctactggatctaagccttccttccctgatgggatgttgattctggttgatcctgaacaagcggttgagcccggggatttctgcattgcaagattaggcggcgacgaatttactttcaaaaaacttattcgcgattcgggacaagttttcctgcaaccactgaacccgcaatacccaatgatcccttgtaatgaatcatgcagtgttgttggtaaggtgatagcttcgcagtggccagaggagacctttgggtaatactagagaaaaaaaaaccccgcttcggcggggtttttttttgatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

SEQ ID NO: 32 - DNA, 11180 bp, synthetic construct
  source 1..11180: mol_type = other DNA
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaaacgcaaaaaaccccgcccctgacagggcggggttttttcgcgtgcgggtgccagggcgtgccctcgagttctctcagttgggggcgtgctattcgatagttgttaaggtcgcggtctgacgctcagtggaacaaccatctgcggtgataaattatctctggcggtgttgacataaataccactggcggtgatactgagcacatgtactcagatccaataggaggaacaatatgcgcgcgcttgttgtgattcgtttatcgcgtgtgacggacgccaccaccagtcccgaacgccaattagaaagctgtcaacaactttgtgctcaacgtggttgggatgtggtgggagtggccgaagaccttgatgtttcgggcgctgttgacccatttgatcgcaaacgtcgtcctaatttggcgcgttggctggcctttgaagaacagcctttcgatgtaattgtcgcatatcgcgttgaccgtctgacacgttccattcgccacctgcaacaacttgtgcattgggcggaagatcataagaaattagttgtttcggcaacagaggcccattttgacaccaccacacctttcgctgctgtagttattgccctgatgggtactgtcgctcaaatggagctcgaggcaattaaggaacgcaatcgctccgcggcccactttaacattcgtgctggcaagtatcgcggcagcttacccccgtggggttatctccccacacgtgtagatggcgaatggcgcctcgtccctgatccggtccaacgcgaacgtattctggaagtctaccatcgtgtagtggataatcatgaacccttacacctcgttgctcatgatctcaatcgccgcggcgttttgagccccaaagattattttgcccaattgcagggtcgtgaaccccaaggtcgcgaatggtccgccacagccttaaaacgctcaatgatttctgaagcaatgcttggctatgcaacgttgaatggcaaaactgttcgtgatgatgatggcgcgcccttggttcgcgccgaaccaattttgacacgcgaacaattagaagctcttcgtgcagaactggttaaaacatcgcgtgccaaaccggccgttagtactccttctcttctccttcgtgtcctgttttgtgctgtctgtggcgaacctgcctataaatttgctggtggcgggcgcaaacatcctcgttatcgttgtcgtagcatgggctttcccaaacattgtggcaatggtactgttgctatggcggaatgggatgccttttgtgaagaacaagtgttagaccttctgggcgatgcagaacgcctcgaaaaggtatgggtcgccgggagcgatagcgcagtggagctggctgaagtaaatgccgaactcgttgatttaactagcttgattggatctcctgcgtatcgcgccggatcgccacaacgtgaggccttagacgcgcgcatcgctgccttagctgcccgccaggaagaactcgaaggtttggaagcgcgtcctagcggatgggaatggcgtgaaactggtcaacgctttggagattggtggcgcgaacaagatacggctgccaagaatacatggctgcgttccatgaatgtgcgtcttacctttgatgtgcgtggtggtttaacccgtacaattgattttggcgacctgcaagaatatgaacaacacctgcgtctgggatcagtcgtggagcgcttgcatacaggcatgtcataaggcatctaactaaaaacaccctaacggcaaaataataaaaaagccggattaataatctggctttttatattctctcactaccatcggcgctacggcgtttcacttctgagttctgcagcggccgctactagtatggtaccattacgccgctacgtcttccgtgccgtcttgagcatcatcctcatcatcatccgttgggggtttagcccaagtaatactcgcacgtttctcaatcggggtgccttggccgcgtccggtcgttgatttggtcaccacaattttatcaacaaacaggcccacaaatacacgtttatcatccacacttgcacggccccaccagcttttcggacctgttgggtccgcatccgcatcttccggaaaccactgatccagcggcaatttgggtgcctcggccgcctcgagctctgccagacgttcctcggcgccctgttgacgtaaggttaatgccgcttgctgtttgcgaaaatgtttacgaccgaccggaccatcatatgccccggccgcacgatcctcatacagctcttcaagggcgttcagcgcatccgcacgttcggccaccagattggcgcgctctcctgatttctccggggcttcggtaagtttaccaaaacggcgcgccgcttcccataacaatgccagggtttcctcatcaccctctgcatgacgaattttattaaagatacgctcggctacaaatttatccagcgcggccatagacacattacaagttccctcatgttggcccggcgccgagggatcaacgactttgcgacggcgacaacgatatgaatctttaattgactcctcgccacgtttggatgtcataaccgcaccgcattcacaataaagtttatccatcgcgcttaagatcgcctgaccacggctcagacctttaccacgaccgcgaccatccagccaggcttgcagttcgtaccattccgcaggttcaataatggggccacagtccagttcaacaggacgcagggtaataggatcacgttggatacgatacccttcgattttggtcgttggggtcccatctggtttcttcttataaatgacttccgcagcaaatccggcgatgcggggatcgcgtaaaatgcgcatcactgtggcggggtcccacgccgagctcgcagttttcttgccgatggtttcaccacgcgttggaaccgcatccgcatccatacgtttgcacaagccggtaattgaaccaggatggatcgccgcctgggaacctggtttaaacggcagatgcttatgagttttaatttcgcgccaccaccaacgaatgacatctggttcaaattcaaacgggcctgtcagcggggttgttgaatgggccagtttattaatcaccacgttaaccatgcgaccattacgggtaatttctttggtttcgctaacaagctcgaagccgtaaggcgctttaccgcccacatatccacccagctcacgttgtaaattcttagtatccaggattttggcgctctttaaactgctctccttgtgtgaggcatccagacgcatgatcaggtgaatcaggtccatgacattgccttggcgaaacacaccctcttgggttgagacgatggtgacacctaacgccagtaactcgcttacgatggggattgcatccatcactttgagacgagaaaagcgagaaacatcgtacacgataatcatatttaagcggcccgcacgacactcatttaaaatacgctcaaattcaggacgttcggctgtaccaaaagcgctggtccctggagcctctgaaaagtgacccacaaaacgaaagcgaccaccatcacgttccacctcacgttgcagatctgcggctttatcctcattcgcggagcgttgggttgcgggggacgcggctgagctgttttcacgttcacgagattggcgatcgtaagcacccgcgtacgtgtccatattgttcctcctattggatctgagtacatgcaaccattatcaccgccagaggtaaaatagtcaacacgcgcggtgttagatatttatcccttgcggtgatattggtaacgaatcagacaattgacggcgacaccatcgaatggcgcaaacctttcgcggtatggcatgatagcgcccggaagagagtcaattcagggtggtgaatgtataagaaggagatatacatatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgactcgagtctggtccaccgctgagcaataactagcataaccccttggggcctctaaacgggtcttgaggggttttttgcgatctgaagcgaaccatgacgttagttcctattccgaagttcctattctctagaaagtataggaacttcgaagcagctcttgttcagaacgctcggtcttgcacaccgggcgttttttctttgtgagtccagcgtcgtggtttgtctggtcaaccaccgcggactcagtggtgtacggtacaaacccagtgccccaactggggtaacctaggagttctctcagttgggggcgtagcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgcttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataactgcaggaggtacccggggatcctctagaaataattttgtttaactttaagaaggagatatacatatgcggggttctcatcatcatcatcatcatggtatggctagcatgactggtggacagcaaatgggtcgggatctgtacgacgatgacgataaggatccgatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcctgcaggatatctggatccacgaagcttcccaccgctgagcaataactagcataaccccttggggcctctaaacgggtattgaggggttttttgccactcactacagcagagcagccacgtagaaagccagtccgcagaaacggtgctgaccccggatgaatgtcagctactgggctatctggacaagggaaaacgcaagcgtttagtttctcgtttcttcttcttccttcatagcttgatccgaaaagttactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatcccaggtcttcacttttgaaaagcattgacaattaatcatcggctcgtataatgtgtggaagtgcgggtgccagggcgtgcccaggggctccccgggcgcgtactccggccggcttgtcgacgacggcggactccgtcgtcaggatcatccgggctctaactaaaaacaccctaacgggtgttttttcttttctggtctcccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagacactgctctttaacaatttatcagatccaataggaggaacaatatgtctaccaaaaaaaaaccacttacacaggaacagttggaagatgcccggcgtcttaaggcgatttatgagaagaagaaaaacgaattagggctgtctcaagaatcagtggctgataagatgggtatgggacagtccggcgtcggggcgttatttaatggtataaacgcattaaatgcctacaacgccgctttactgaccaaaattttgaaagtctccgtagaagaatttagtccatctattgctagagagatatacgagatgtacgaagccgttagtatgcaaccatcgctgcgttcggagtatgaatatccagtcttttctcatgtccaagccggaatgttttcccccgagttgcggacatttacgaagggagacgcagagcgttgggtgtcgacaactaagaaagcctccgactctgccttctggctggaggtagaaggaaactctatgaccgcccctactggatctaagccttccttccctgatgggatgttgattctggttgatcctgaacaagcggttgagcccggggatttctgcattgcaagattaggcggcgacgaatttactttcaaaaaacttattcgcgattcgggacaagttttcctgcaaccactgaacccgcaatacccaatgatcccttgtaatgaatcatgcagtgttgttggtaaggtgatagcttcgcagtggccagaggagacctttgggtaatactagagaaaaaaaaaccccgcttcggcggggtttttttttgatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

SEQ ID NO: 33 - DNA, 10123 bp, synthetic construct
  source 1..10123: mol_type = other DNA
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaaacgcaaaaaaccccgcccctgacagggcggggttttttcgcgtgcgggtgccagggcgtgccctcgagttctctcagttgggggcgtgctattcgatagttgttaaggtcgcggtctgacgctcagtggaacaaccatctgcggtgataaattatctctggcggtgttgacataaataccactggcggtgatactgagcacatgtactcagatccaataggaggaacaatatgcgcgcgcttgttgtgattcgtttatcgcgtgtgacggacgccaccaccagtcccgaacgccaattagaaagctgtcaacaactttgtgctcaacgtggttgggatgtggtgggagtggccgaagaccttgatgtttcgggcgctgttgacccatttgatcgcaaacgtcgtcctaatttggcgcgttggctggcctttgaagaacagcctttcgatgtaattgtcgcatatcgcgttgaccgtctgacacgttccattcgccacctgcaacaacttgtgcattgggcggaagatcataagaaattagttgtttcggcaacagaggcccattttgacaccaccacacctttcgctgctgtagttattgccctgatgggtactgtcgctcaaatggagctcgaggcaattaaggaacgcaatcgctccgcggcccactttaacattcgtgctggcaagtatcgcggcagcttacccccgtggggttatctccccacacgtgtagatggcgaatggcgcctcgtccctgatccggtccaacgcgaacgtattctggaagtctaccatcgtgtagtggataatcatgaacccttacacctcgttgctcatgatctcaatcgccgcggcgttttgagccccaaagattattttgcccaattgcagggtcgtgaaccccaaggtcgcgaatggtccgccacagccttaaaacgctcaatgatttctgaagcaatgcttggctatgcaacgttgaatggcaaaactgttcgtgatgatgatggcgcgcccttggttcgcgccgaaccaattttgacacgcgaacaattagaagctcttcgtgcagaactggttaaaacatcgcgtgccaaaccggccgttagtactccttctcttctccttcgtgtcctgttttgtgctgtctgtggcgaacctgcctataaatttgctggtggcgggcgcaaacatcctcgttatcgttgtcgtagcatgggctttcccaaacattgtggcaatggtactgttgctatggcggaatgggatgccttttgtgaagaacaagtgttagaccttctgggcgatgcagaacgcctcgaaaaggtatgggtcgccgggagcgatagcgcagtggagctggctgaagtaaatgccgaactcgttgatttaactagcttgattggatctcctgcgtatcgcgccggatcgccacaacgtgaggccttagacgcgcgcatcgctgccttagctgcccgccaggaagaactcgaaggtttggaagcgcgtcctagcggatgggaatggcgtgaaactggtcaacgctttggagattggtggcgcgaacaagatacggctgccaagaatacatggctgcgttccatgaatgtgcgtcttacctttgatgtgcgtggtggtttaacccgtacaattgattttggcgacctgcaagaatatgaacaacacctgcgtctgggatcagtcgtggagcgcttgcatacaggcatgtcataaggcatctaactaaaaacaccctaacggcaaaataataaaaaagccggattaataatctggctttttatattctctcactaccatcggcgctacggcgtttcacttctgagttctgcagcggccgctactagtatggtaccattacgccgctacgtcttccgtgccgtcttgagcatcatcctcatcatcatccgttgggggtttagcccaagtaatactcgcacgtttctcaatcggggtgccttggccgcgtccggtcgttgatttggtcaccacaattttatcaacaaacaggcccacaaatacacgtttatcatccacacttgcacggccccaccagcttttcggacctgttgggtccgcatccgcatcttccggaaaccactgatccagcggcaatttgggtgcctcggccgcctcgagctctgccagacgttcctcggcgccctgttgacgtaaggttaatgccgcttgctgtttgcgaaaatgtttacgaccgaccggaccatcatatgccccggccgcacgatcctcatacagctcttcaagggcgttcagcgcatccgcacgttcggccaccagattggcgcgctctcctgatttctccggggcttcggtaagtttaccaaaacggcgcgccgcttcccataacaatgccagggtttcctcatcaccctctgcatgacgaattttattaaagatacgctcggctacaaatttatccagcgcggccatagacacattacaagttccctcatgttggcccggcgccgagggatcaacgactttgcgacggcgacaacgatatgaatctttaattgactcctcgccacgtttggatgtcataaccgcaccgcattcacaataaagtttatccatcgcgcttaagatcgcctgaccacggctcagacctttaccacgaccgcgaccatccagccaggcttgcagttcgtaccattccgcaggttcaataatggggccacagtccagttcaacaggacgcagggtaataggatcacgttggatacgatacccttcgattttggtcgttggggtcccatctggtttcttcttataaatgacttccgcagcaaatccggcgatgcggggatcgcgtaaaatgcgcatcactgtggcggggtcccacgccgagctcgcagttttcttgccgatggtttcaccacgcgttggaaccgcatccgcatccatacgtttgcacaagccggtaattgaaccaggatggatcgccgcctgggaacctggtttaaacggcagatgcttatgagttttaatttcgcgccaccaccaacgaatgacatctggttcaaattcaaacgggcctgtcagcggggttgttgaatgggccagtttattaatcaccacgttaaccatgcgaccattacgggtaatttctttggtttcgctaacaagctcgaagccgtaaggcgctttaccgcccacatatccacccagctcacgttgtaaattcttagtatccaggattttggcgctctttaaactgctctccttgtgtgaggcatccagacgcatgatcaggtgaatcaggtccatgacattgccttggcgaaacacaccctcttgggttgagacgatggtgacacctaacgccagtaactcgcttacgatggggattgcatccatcactttgagacgagaaaagcgagaaacatcgtacacgataatcatatttaagcggcccgcacgacactcatttaaaatacgctcaaattcaggacgttcggctgtaccaaaagcgctggtccctggagcctctgaaaagtgacccacaaaacgaaagcgaccaccatcacgttccacctcacgttgcagatctgcggctttatcctcattcgcggagcgttgggttgcgggggacgcggctgagctgttttcacgttcacgagattggcgatcgtaagcacccgcgtacgtgtccatattgttcctcctattggatctgagtacatgcaaccattatcaccgccagaggtaaaatagtcaacacgcgcggtgttagatatttatcccttgcggtgatattggtaacgaatcagacaattgacggcgacaccatcgaatggcgcaaacctttcgcggtatggcatgatagcgcccggaagagagtcaattcagggtggtgaatgtataagaaggagatatacatatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgactcgagtctggtccaccgctgagcaataactagcataaccccttggggcctctaaacgggtcttgaggggttttttgcgatctgaagcgaaccatgacgttagttcctattccgaagttcctattctctagaaagtataggaacttcgaagcagctcttgttcagaacgctcggtcttgcacaccgggcgttttttctttgtgagtccagcgtcgtggtttgtctggtcaaccaccgcggactcagtggtgtacggtacaaacccagtgccccaactggggtaacctaggagttctctcagttgggggcgtagcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgaaaaaccccgccctgtcaggggcggggtttttggcgtacgtctcgaggtcgacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataactgcaggaggtacccggggatcctctagaaataattttgtttaactttaagaaggagatatacatatgcggggttctcatcatcatcatcatcatggtatggctagcatgactggtggacagcaaatgggtcgggatctgtacgacgatgacgataaggatccgatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcctgcaggatatctggatccacgaagcttcccaccgctgagcaataactagcataaccccttggggcctctaaacgggtattgaggggttttttgccactcactacagcagagcagccacgtagaaagccagtccgcagaaacggtgctgaccccggatgaatgtcagctactgggctatctggacaagggaaaacgcaagcgtttagtttctcgtttcttcttcttccttcatagcttgatccgaaaagttactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatcccaggtcttcacttttgaaaagcattgacaattaatcatcggctcgtataatgtgtggaagtgcgggtgccagggcgtgcccaggggctccccgggcgcgtactccggccggcttgtcgacgacggcggactccgtcgtcaggatcatccgggctctaactaaaaacaccctaacgggtgttttttcttttctggtctcccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagacactgctctttaacaatttatcagatccaataggaggaacaatatgtctaccaaaaaaaaaccacttacacaggaacagttggaagatgcccggcgtcttaaggcgatttatgagaagaagaaaaacgaattagggctgtctcaagaatcagtggctgataagatgggtatgggacagtccggcgtcggggcgttatttaatggtataaacgcattaaatgcctacaacgccgctttactgaccaaaattttgaaagtctccgtagaagaatttagtccatctattgctagagagatatacgagatgtacgaagccgttagtatgcaaccatcgctgcgttcggagtatgaatatccagtcttttctcatgtccaagccggaatgttttcccccgagttgcggacatttacgaagggagacgcagagcgttgggtgtcgacaactaagaaagcctccgactctgccttctggctggaggtagaaggaaactctatgaccgcccctactggatctaagccttccttccctgatgggatgttgattctggttgatcctgaacaagcggttgagcccggggatttctgcattgcaagattaggcggcgacgaatttactttcaaaaaacttattcgcgattcgggacaagttttcctgcaaccactgaacccgcaatacccaatgatcccttgtaatgaatcatgcagtgttgttggtaaggtgatagcttcgcagtggccagaggagacctttgggtaatactagagaaaaaaaaaccccgcttcggcggggtttttttttgatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

SEQ ID NO: 34 - DNA, 5055 bp, synthetic construct
  source 1..5055: mol_type = other DNA
gggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggcccgctaacgcggcaccccatccccccaggggctgcgcccctcggccgcgaacgacctcaccccaaaaatggcagccacgtagaaagccagtccgcagaaacggtgctgaccccggatgaatgtcagctactgggctatctggacaagggaaaacgcaagcgaaaagcattgacaattaatcatcggctcgtataatgtgtggaaagtgccccaactggggtaacctttgagttctctcagttgggggcgtttaataaactatggaagtatgtacagtcttgcaatgttgagtgaacaaacttccataataaaattgcgagtttttatttcgtttatttcaattaaggtaactaaaaaactcctttacgtcgtggtttgtctggtcaaccaccgcggtctccgtcgtcaggatcatccgggcatgccaacacaattaacatctcaatcaaggtaaatgctttttgctttttttgccaatcatcagataactatggcggcacgtgcattaaccacggttgtatcccgtctaaagtactcgtgtgcgggtgccagggcgtgcccttgggctccccgggcgcgtactccggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatagtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctaataagcggaaaaaccccgccctgtcaggggcggggtttttggcgtacgtctcgaggtcgacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataactgcaggaggtacccggggatcctctagaaataattttgtttaactttaagaaggagatatacatatgcggggttctcatcatcatcatcatcatggtatggctagcatgactggtggacagcaaatgggtcgggatctgtacgacgatgacgataaggatccgatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaattcctgcaggatatctggatccacgaagcttcccaccgctgagcaataactagcataaccccttggggcctctaaacgggtattgaggggttttttgccactcactacagcagagccatgta

SEQ ID NO: 35 - DNA, 526 bp, synthetic construct
  source 1..526: mol_type = other DNA
gggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccgg

SEQ ID NO: 36 - DNA, 10177 bp, synthetic construct
  source 1..10177: mol_type = other DNA
aacgtcccaacggtgagcacgacggctttggcacggaacttcagtcccatttgggtaacagcaccgaccacgcgatcgttttcgacaataagatcttcaaccgcctgctggaagatcatcaggttcggttggttctccagcgccgtacgtaccgcctgacggtagagcacacgatccgcctgagctcgggtagcgcgaaccgccggtcctttgcttgcgtttagtatcctaaactggatacccgcctgatcgatcgctttcgccatcagaccgccgagtgcatccacttcttttaccagatgtcccttcccaataccgccgatcgccgggttgcagctcatctgccccagagtgtcgatattgtgtgtcaaaagcagagtctgttgacccatacgcgccgcggccatcgcggcctcggtgcctgcatgacccccgccaatgatgatgacgtcaaaaggatccggataaaacatggtgattgcctcgcataacgcggtatgaaaatggattgaagcccgggccgtggattctactcaactttgtcggcttgagaaacgcaaaaaaccccgcccctgacagggcggggttttttcgcgtgcgggtgccagggcgtgccctcgagttctctcagttgggggcgtgctattcgatagttgttaaggtcgcggtctgacgctcagtggaacaaccatctgcggtgataaattatctctggcggtgttgacataaataccactggcggtgatactgagcacatgtactcagatccaataggaggaacaatatgcgcgcgcttgttgtgattcgtttatcgcgtgtgacggacgccaccaccagtcccgaacgccaattagaaagctgtcaacaactttgtgctcaacgtggttgggatgtggtgggagtggccgaagaccttgatgtttcgggcgctgttgacccatttgatcgcaaacgtcgtcctaatttggcgcgttggctggcctttgaagaacagcctttcgatgtaattgtcgcatatcgcgttgaccgtctgacacgttccattcgccacctgcaacaacttgtgcattgggcggaagatcataagaaattagttgtttcggcaacagaggcccattttgacaccaccacacctttcgctgctgtagttattgccctgatgggtactgtcgctcaaatggagctcgaggcaattaaggaacgcaatcgctccgcggcccactttaacattcgtgctggcaagtatcgcggcagcttacccccgtggggttatctccccacacgtgtagatggcgaatggcgcctcgtccctgatccggtccaacgcgaacgtattctggaagtctaccatcgtgtagtggataatcatgaacccttacacctcgttgctcatgatctcaatcgccgcggcgttttgagccccaaagattattttgcccaattgcagggtcgtgaaccccaaggtcgcgaatggtccgccacagccttaaaacgctcaatgatttctgaagcaatgcttggctatgcaacgttgaatggcaaaactgttcgtgatgatgatggcgcgcccttggttcgcgccgaaccaattttgacacgcgaacaattagaagctcttcgtgcagaactggttaaaacatcgcgtgccaaaccggccgttagtactccttctcttctccttcgtgtcctgttttgtgctgtctgtggcgaacctgcctataaatttgctggtggcgggcgcaaacatcctcgttatcgttgtcgtagcatgggctttcccaaacattgtggcaatggtactgttgctatggcggaatgggatgccttttgtgaagaacaagtgttagaccttctgggcgatgcagaacgcctcgaaaaggtatgggtcgccgggagcgatagcgcagtggagctggctgaagtaaatgccgaactcgttgatttaactagcttgattggatctcctgcgtatcgcgccggatcgccacaacgtgaggccttagacgcgcgcatcgctgccttagctgcccgccaggaagaactcgaaggtttggaagcgcgtcctagcggatgggaatggcgtgaaactggtcaacgctttggagattggtggcgcgaacaagatacggctgccaagaatacatggctgcgttccatgaatgtgcgtcttacctttgatgtgcgtggtggtttaacccgtacaattgattttggcgacctgcaagaatatgaacaacacctgcgtctgggatcagtcgtggagcgcttgcatacaggcatgtcataaggcatctaactaaaaacaccctaacggcaaaataataaaaaagccggattaataatctggctttttatattctctcactaccatcggcgctacggcgtttcacttctgagttctgcagcggccgctactagtatggtaccattacgccgctacgtcttccgtgccgtcttgagcatcatcctcatcatcatccgttgggggtttagcccaagtaatactcgcacgtttctcaatcggggtgccttggccgcgtccggtcgttgatttggtcaccacaattttatcaacaaacaggcccacaaatacacgtttatcatccacacttgcacggccccaccagcttttcggacctgttgggtccgcatccgcatcttccggaaaccactgatccagcggcaatttgggtgcctcggccgcctcgagctctgccagacgttcctcggcgccctgttgacgtaaggttaatgccgcttgctgtttgcgaaaatgtttacgaccgaccggaccatcatatgccccggccgcacgatcctcatacagctcttcaagggcgttcagcgcatccgcacgttcggccaccagattggcgcgctctcctgatttctccggggcttcggtaagtttaccaaaacggcgcgccgcttcccataacaatgccagggtttcctcatcaccctctgcatgacgaattttattaaagatacgctcggctacaaatttatccagcgcggccatagacacattacaagttccctcatgttggcccggcgccgagggatcaacgactttgcgacggcgacaacgatatgaatctttaattgactcctcgccacgtttggatgtcataaccgcaccgcattcacaataaagtttatccatcgcgcttaagatcgcctgaccacggctcagacctttaccacgaccgcgaccatccagccaggcttgcagttcgtaccattccgcaggttcaataatggggccacagtccagttcaacaggacgcagggtaataggatcacgttggatacgatacccttcgattttggtcgttggggtcccatctggtttcttcttataaatgacttccgcagcaaatccggcgatgcggggatcgcgtaaaatgcgcatcactgtggcggggtcccacgccgagctcgcagttttcttgccgatggtttcaccacgcgttggaaccgcatccgcatccatacgtttgcacaagccggtaattgaaccaggatggatcgccgcctgggaacctggtttaaacggcagatgcttatgagttttaatttcgcgccaccaccaacgaatgacatctggttcaaattcaaacgggcctgtcagcggggttgttgaatgggccagtttattaatcaccacgttaaccatgcgaccattacgggtaatttctttggtttcgctaacaagctcgaagccgtaaggcgctttaccgcccacatatccacccagctcacgttgtaaattcttagtatccaggattttggcgctctttaaactgctctccttgtgtgaggcatccagacgcatgatcaggtgaatcaggtccatgacattgccttggcgaaacacaccctcttgggttgagacgatggtgacacctaacgccagtaactcgcttacgatggggattgcatccatcactttgagacgagaaaagcgagaaacatcgtacacgataatcatatttaagcggcccgcacgacactcatttaaaatacgctcaaattcaggacgttcggctgtaccaaaagcgctggtccctggagcctctgaaaagtgacccacaaaacgaaagcgaccaccatcacgttccacctcacgttgcagatctgcggctttatcctcattcgcggagcgttgggttgcgggggacgcggctgagctgttttcacgttcacgagattggcgatcgtaagcacccgcgtacgtgtccatattgttcctcctattggatctgagtacatgcaaccattatcaccgccagaggtaaaatagtcaacacgcgcggtgttagatatttatcccttgcggtgatattggtaacgaatcagacaattgacggcgacaccatcgaatggcgcaaacctttcgcggtatggcatgatagcgcccggaagagagtcaattcagggtggtgaatgtataagaaggagatatacatatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgactcgagtctggtccaccgctgagcaataactagcataaccccttggggcctctaaacgggtcttgaggggttttttgcgatctgaagcgaaccatgacgttagttcctattccgaagttcctattctctaggtataggaacttcgaagcagctcttgttcagaacgctcggtcttgcacaccgggcgttttttctttgtgagtccagcgtcgtggtttgtctggtcaaccaccgcggactcagtggtgtacggtacaaacccagtgccccaactggggtaacctaggagttctctcagttgggggcgtaacaattgggatccactgctctttaacaatttatcagatccaataggaggaacaatatgcggggttctcatcatcatcatcatcatggtatggctagcatgactggtggacagcaaatgggtcgggatctgtacgacgatgacgataaggatccgatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggcggaaaaaccccgccctgtcaggggcggggtttttggcgtacgtctcgaggtcgacgccactcactacagcagagccatgtagggccgccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggcccgctaacgcggcaccccatccccccaggggctgcgcccctcggccgcgaacgacctcaccccaaaaatggcagccacgtagaaagccagtccgcagaaacggtgctgaccccggatgaatgtcagctactgggctatctggacaagggaaaacgcaagcgtttagtttctcgtttcttcttcttccttcatagcttgatccgaaaagttactgtggataactctgtcaggaagcttggatcaaccggtagttatccaaagaacaactgttgttcagtttttgagttgtgtataacccctcattctgatcccagcttatacggtccaggatcaccgatcattcacagttaatgatcctttccaggttgttgatcttaaaagccggatccttgttatccacagggcagtgcgatcctaataagagatcacaatagaacagatctctaaataaatagatcttctttttaatacccaggatcccaggtcttcacttttgaaaagcattgacaattaatcatcggctcgtataatgtgtggaagtgcgggtgccagggcgtgcccaggggctccccgggcgcgtactccggccggcttgtcgacgacggcggactccgtcgtcaggatcatccgggctctaactaaaaacaccctaacgggtgttttttcttttctggtctcccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagacactgctctttaacaatttatcagatccaataggaggaacaatatgtctaccaaaaaaaaaccacttacacaggaacagttggaagatgcccggcgtcttaaggcgatttatgagaagaagaaaaacgaattagggctgtctcaagaatcagtggctgataagatgggtatgggacagtccggcgtcggggcgttatttaatggtataaacgcattaaatgcctacaacgccgctttactgaccaaaattttgaaagtctccgtagaagaatttagtccatctattgctagagagatatacgagatgtacgaagccgttagtatgcaaccatcgctgcgttcggagtatgaatatccagtcttttctcatgtccaagccggaatgttttcccccgagttgcggacatttacgaagggagacgcagagcgttgggtgtcgacaactaagaaagcctccgactctgccttctggctggaggtagaaggaaactctatgaccgcccctactggatctaagccttccttccctgatgggatgttgattctggttgatcctgaacaagcggttgagcccggggatttctgcattgcaagattaggcggcgacgaatttactttcaaaaaacttattcgcgattcgggacaagttttcctgcaaccactgaacccgcaatacccaatgatcccttgtaatgaatcatgcagtgttgttggtaaggtgatagcttcgcagtggccagaggagacctttgggtaatactagagaaaaaaaaaccccgcttcggcggggtttttttttgatcgcacgatctgtatacttatttgagtaaattaacccacgatcccagccattcttctgccggatcttccggaatgtcgtgatcaagaatgttgatcttcagtgtttcgcctgtctgttttgcaccggaatttttgagttctgcctcgagtttatcgatagccccacaaaaggtgtcatattcacgactgccaataccgattgcgccaaagcggactgcagaaagatcgggcttctgttcctgcaatgcttcatagaaaggagaaaggttgtccggaatatctccggcaccgtgggtggagctgataaccagccagatccctgaggcaggtaaatcttctaacagcggaccgtgcagcgtttcggtggtaaaacccgcctcttccagcttttcagccaggtgttctgctacatattcggcaccgccgagggtgctgccgctgataagagtgatatctgccat

(19) United States
(12) Patent Application Publication
(10) Pub. No.: US 2026/0275360 A1
(43) Pub. Date: Sep. 17, 2026

(54) METHOD OF BIOSYNTHESIS

(71) Applicant: OÜ GEARBOX BIOSCIENCES, Tartu (EE)

(72) Inventors: Arvi JÕERS, Tartu (EE); Villu KASARI, Tartu (EE); Marje KASARI, Tartu (EE)

(21) Appl. No.: 19/168,791

(22) PCT Filed: Mar. 28, 2024
(86) PCT No.: PCT/EP2024/058636

§ 371 Date: Sep. 24, 2025

(30) Foreign Application Priority Data
Mar. 28, 2023 (GB) 2304540.4

(51) Int. Cl. C12N 15/70; C12N 1/20

(52) U.S. Cl. CPC C12N 15/70; C12N 1/20

(57) ABSTRACT

The present invention relates generally to methods of producing products by cells and in particular to the field of industrial biotechnology or microbiology where living cells are used to produce useful chemicals and products, e.g. biological products such as proteins. The present invention relates to a method of producing a product by a cell, the method comprising: excising one or more nucleic acid sequences from a chromosome in the cell, wherein the one or more nucleic acid sequences comprises a gene of interest and an origin of replication; forming an extra-chromosomal DNA molecule, wherein the extra-chromosomal DNA molecule comprises the excised gene of interest and an excised origin of replication; and inducing expression of the gene of interest from the extra-chromosomal DNA molecule. The present invention also relates to cells, polynucleotide vectors, and uses thereof for producing products.

The present invention relates generally to methods of producing products by cells and in particular to the field of industrial biotechnology or microbiology where living cells are used to produce useful chemicals and products, e.g. biological products such as proteins.

Recombinant proteins are an important class of products that are used in many applications including food processing, therapeutics, diagnostics, chemical synthesis etc. For practical reasons recombinant proteins must be synthesised by living cells. Both prokaryotic and eukaryotic cells are used for this, depending on the nature of the product. Gram-negative bacterium Escherichia coli (E. coli) is one of the most widely used protein production hosts and many commercially available recombinant proteins are produced in E. coli.

A typical protein production endeavour in E. coli starts with a cloning of a gene-of-interest (GOI) into a plasmid. Plasmids are autonomously replicating DNA molecules that can be present in bacteria (and in some eukaryotes) in multiple copies. This is beneficial in protein production—having multiple copies of GOI present in a cell intrinsically results in a higher expression level of protein-of-interest (POI). This gives plasmid-based protein expression systems a clear advantage over methods where GOI is present in the genome as a single copy.

To ensure that plasmids are not lost during the production, their presence must be selected for. By far, the most common way to do that is to use antibiotics. In this setup, plasmids carry an antibiotic resistance gene and antibiotics are added into the growth medium. As a result, only plasmid-containing cells can grow.

There are several problems associated with the use of antibiotics in protein production. Some antibiotics are degraded or inactivated so they must be resupplied during the cultivation to maintain an efficient selection. In some situations antibiotics must be removed during the protein purification and this can add additional purification steps, increasing the cost and decreasing protein yield. Removal of antibiotics is crucial if the protein is used in preparation of human food (food enzymes) or as pharmaceuticals (therapeutic proteins). Humans can be allergic to some antibiotics and their unintentional use can damage normal microflora. In addition, the final product must also be free of antibiotic resistance genes that could spread through horizontal gene transfer. These requirements have led to establishment of strict quality control mechanisms by regulatory agencies and encouragement to abandon the use of antibiotics in protein production. Many protein production companies want to abandon the use of antibiotics in large-scale productions altogether.

Several alternative selection mechanisms have been developed. One group of methods uses episomal complementation of auxotrophic strains to stabilise the plasmids. In this case genes encoding for essential metabolic functions are inactivated in the genome and re-introduced on the plasmid. This makes the presence of plasmid essential for growth, if the metabolite cannot be acquired otherwise, and the GOI is maintained on the same plasmid. The patent EP0284126B1 mentions genes leu, his, trp or the like as auxotrophic selection markers. The U.S. Pat. No. 8,465,946B2 discloses a selection system using a glycerine-3-phosphate dehydrogenase gene as auxotrophic marker and the patent EP0185512B1 discloses dal gene as auxotrophic marker.

The use of such auxotrophic markers in industrial fermentation is, however, difficult, as almost all necessary substances (amino acids, etc.) are present in industrial fermentation media. Industrial medium contains undefined complex mixtures like corn steep liquor and yeast extract, and cells can balance their inability to synthesise a certain metabolite by absorption of this metabolite from the culture medium. The use of chemically defined medium, consisting of only pure components, is in most cases prohibitively expensive.

Another known approach is to use toxin-antitoxin pairs for antibiotic-free selection of plasmids. The toxin gene is usually incorporated into host chromosomal DNA and the antitoxin gene is carried on the plasmid. When both toxin and antitoxin are expressed in the cells the antitoxin binding neutralises toxin and cells can grow. If the plasmid is lost from the cells the antitoxin level decreases, toxin escapes the neutralisation and inhibits the cell growth. Such a system is described for example in the U.S. Pat. Nos. 8,470,580B2 and 10,718,001B2, both utilizing CcdA/CcdB toxin-antitoxin system. The drawbacks of such a system is that it is cumbersome to handle as the toxin-expressing cells must always contain the antitoxin-expressing plasmid and thus the introduction of a new plasmid with GOI must be accompanied with removing the previously existing one. Also the strength of the selection can vary depending on the expression level of toxin and antitoxin.

Another selectable system is RNA selectable marker where RNA, produced from a plasmid, represses a toxic gene encoded in the genome. An example of such a system is described in the U.S. Pat. No. 10,144,935B2.

As an alternative to plasmid-based expression, GOI can be incorporated into the genome. In this case the whole setup is very stable and does not require any selection. The disadvantage of this method is that GOI is present only in a single copy and thus the expression level is low compared to multi-copy plasmids. It is possible to amplify the chromosomally integrated gene [1], [2], resulting in tandem array of GOI. Such a setup is, however, unstable and often relaxes back to the single copy [3].

The patent application EP0284126B1 discloses a method of multicopy integration where an essential gene is between two copies of GOI. The patent application WO1996023073A1 describes a method of random insertion of GOI together with a removable marker gene to the bacterial genome and subsequent removal of the marker gene (an antibiotic resistance gene). Repeating that procedure allows insertion of multiple copies of GOI. However, these one-by-one approaches are very laborious if it is desired to achieve copy-numbers like plasmids have (>20).

There are several variants of the methods mentioned above, many of them reviewed in [4] and [5].

Changing the GOI copy-number in living cells has been described using plasmids with two replication origins [6]. During plasmid maintenance, only the low copy-number origin of replication (oriS) is active and plasmid is present in a few copies per cell. When the higher copy-number oriV is activated the plasmid copy-number increases. This has been used to increase the yield of plasmid [6] or protein [7] encoded by it. However, the GOI stays on the plasmid all the time and a constant presence of antibiotic is required for selection of plasmid maintenance. Other systems are also described in the art where constant presence of antibiotic is required [8], [9].

Therefore, there exists a need for a method of expressing a gene of interest at high level in a host cell without the use of selection markers such as antibiotic resistance.

Here we describe a novel expression system which involves only a single GOI integration into a pre-determined location in the genome, does not require any selection (antibiotic or any other) during growth and production, and provides high level protein expression rivalling and exceeding the ones from multi-copy plasmids.

The solution provided herein is most suitable for use in a two-stage bioproduction process. In a two-stage bioprocess, the bioprocess is divided into two stages—at first the cells grow at maximum rate without (significant) production and, when enough biomass has accumulated, product production is induced. During the growth stage, the GOI is integrated (i.e. present) in the genome in a single copy. Such arrangement is very stable and does not require any selection. When the biomass has accumulated to the desired level, site-specific recombinases are induced in the cells. GOI together with surrounding sequences are excised from the genome and circularized, forming a fully functional plasmid. This plasmid will start to replicate and its copy-number increases. Importantly, no selection or antibiotic resistance gene is necessary because plasmid formation takes place late in growth stage and there are not enough cell divisions left to lose the plasmid from cells. When induced, the product, or protein of interest (POI), is expressed at high level, similar or exceeding the expression from conventional expression plasmids.

Thus, in one aspect, the invention provides a method of producing a product by a cell, the method comprising:
excising one or more nucleic acid sequences from a chromosome in the cell, wherein the one or more nucleic acid sequences comprises a gene of interest and an origin of replication; and
forming an extra-chromosomal DNA molecule, wherein the extra-chromosomal DNA molecule comprises the excised gene of interest and an (or the) excised origin of replication.

In embodiments, the invention provides a method of producing a product by a cell, the method comprising:
excising one or more nucleic acid sequences from a chromosome in the cell, wherein the one or more nucleic acid sequences comprises a gene of interest and an origin of replication;
forming an extra-chromosomal DNA molecule, wherein the extra-chromosomal DNA molecule comprises the excised gene of interest and an (or the) excised origin of replication; and
inducing expression of the gene of interest from the extra-chromosomal DNA molecule.

In embodiments, the expression of the gene of interest from the extra-chromosomal DNA molecule is induced by formation of the extra-chromosomal DNA molecule.

In embodiments, the expression of the gene of interest from the extra-chromosomal DNA molecule is induced at the same time as, or after, formation of the extra-chromosomal DNA molecule.

The step of inducing the expression of the gene of interest from the extra-chromosomal DNA molecule typically refers to an event or act which triggers the expression (or increased expression) of the gene of interest when the gene of interest is part of the extra-chromosomal DNA molecule. Thus, said step encompasses setups where the act of administration or introduction of an agent (e.g. chemical inducer) or condition (e.g. temperature change) leading to the induction of expression of the gene of interest takes place before, or at the same time as, or after, the excising and forming steps have taken place. Thus, the act leading to the induction of expression of the gene of interest (e.g. the addition of a chemical inducer for example to the cell culture medium, or the changing of temperature for example of the cell culture medium) may take place even before the excising and forming steps take place.

This may be done for example in setups where the expression of the gene of interest is not possible when the gene of interest is part of the chromosome (for example because the gene of interest is not operatively linked to a promoter when in the chromosomal arrangement), but is possible when the gene of interest is part of the extra-chromosomal molecule (for example because the gene of interest becomes operatively linked to a promoter through formation of the extra-chromosomal DNA molecule). In such setups, the act leading to the induction of expression of the gene of interest may take place before the excising and forming steps take place, in which case the inducing of expression of the gene of interest from the extra-chromosomal DNA molecule may take place at the same time as, or after (e.g. shortly or immediately after), the extra-chromosomal DNA molecule is formed.

It also may be possible to induce expression of the gene of interest from the extra-chromosomal DNA molecule at the same time as, or after, the extra-chromosomal DNA molecule is formed, by administering or introducing an agent (e.g. chemical inducer) or condition (e.g. temperature change) at the same time as, or after, the extra-chromosomal DNA molecule is formed (e.g. immediately after the extra-chromosomal DNA molecule is formed, or shortly after the extra-chromosomal DNA molecule is formed, or later than this, for example after the extra-chromosomal DNA molecule has been replicated). In such cases, the inducing of expression of the gene of interest from the extra-chromosomal DNA molecule may take place when the agent or condition are administered or introduced.

The above principles explained in relation to the step of “inducing the expression of the gene of interest from the extra-chromosomal DNA molecule” may be applied to the step of inducing replication of the extra-chromosomal DNA molecule, or any other induction step in the method herein, mutatis mutandis.

In other embodiments described in more detail elsewhere herein, it is also possible to induce expression from the extra-chromosomal DNA molecule by formation of the extra-chromosomal DNA molecule, for example by bringing about an operative linkage between an appropriate promoter and the gene of interest when the extra-chromosomal DNA molecule is formed.

The steps of the methods of the invention may be carried out in any appropriate order. Preferably, the steps of the methods of the invention are carried out in the order in which they are listed.

The steps of excising, forming, replicating and expressing as recited herein can all take place intracellularly in the same cell.

The term “origin of replication” is a term of the art and is understood by the skilled person. It refers generally to any sequence, e.g. nucleotide sequence, at which DNA replication can be initiated, for example by the binding of appropriate proteins and enzymes that are part of the replication machinery or necessary for the replication process. The origin of replication in a particular cell or cell line (e.g. a bacterial strain) may be referred to in the art by a specific name. For example, the origin of replication as found in the chromosome of Escherichia coli is referred to as oriC.

The extra-chromosomal DNA molecule as described herein is typically a double-stranded DNA molecule. The extra-chromosomal DNA molecule is formed from nucleic acid sequence(s) excised from the chromosome. The extra-chromosomal DNA molecule comprises a gene of interest and an origin of replication. The origin of replication, of course, functions to enable the replication of the extra-chromosomal DNA molecule within the cell. The replication machinery which cooperates with the origin of replication to cause replication of the extra-chromosomal DNA molecule may be located at any appropriate location within the cell, for example can be located on the extra-chromosomal DNA molecule excised from the chromosome (or on another extra-chromosomal DNA molecule), on the chromosome, or a combination of these, e.g. on both the extra-chromosomal DNA molecule excised from the chromosome and the chromosome. The gene of interest is arranged on the extra-chromosomal DNA molecule so as to facilitate or enable expression of the gene of interest. Thus, the function of the extra-chromosomal DNA molecule is to have the ability to be replicated (to increase copy number) and express the gene of interest, thus enabling high expression of the gene of interest. The extra-chromosomal DNA molecule may be linear or circular (or circularized). The extra-chromosomal DNA molecule may be a plasmid, for example a circular or linear plasmid, more preferably a circular plasmid.

The extra-chromosomal DNA molecule is formed from one or more of the one or more nucleic acid sequences that are excised from the chromosome during the method of the invention.

The excising step may comprise the excising of multiple nucleic acid sequences from the chromosome, wherein these multiple nucleic acid sequences are then joined (for example by ligation, for example by the action of DNA ligase) so as to form the extra-chromosomal DNA molecule. The multiple nucleic acid sequences together comprise a gene of interest and an origin of replication. Thus, the gene of interest and the origin of replication may be located on (or within) different such nucleic acid sequences.

Thus, in scenarios where the excising step involves the excising of multiple nucleic acid sequences from the chromosome, the excision (or excision event or excision reaction or excision process) of the origin of replication from the chromosome may be different to the excision (or excision event or excision reaction or excision process) of the gene of interest from the chromosome. In this scenario, the excised nucleic acid sequence comprising the gene of interest may then be joined (for example by the action of DNA ligase) with the excised nucleic acid sequence comprising an (or the) origin of replication (and optionally other excised nucleic acid sequences, where three or more nucleic acid sequences are excised) in order to form the extra-chromosomal DNA molecule.

Alternatively and more preferably, the one or more nucleic acid sequences is one nucleic acid sequence. Here, the excising step involves the excising of a single nucleic acid sequence, e.g. the excising step may consist of the excision of a single nucleic acid sequence from the chromosome, and the extra-chromosomal DNA molecule may consist of said single nucleic acid sequence, i.e. the singly excised nucleic acid sequence. Thus, in such embodiments, the gene of interest is excised as part of the same DNA molecule as an (or the) origin of replication. Alternatively viewed, the gene of interest is excised from the chromosome through the same excision (or excision event, excision reaction or excision process) as the excision of an (or the) origin of replication. Alternatively viewed, in the excising step, the gene of interest and origin of replication are excised as a single (or one, i.e. only one) DNA molecule. Thus, as a result of the excising step, the gene of interest and the origin of replication exist extra-chromosomally (e.g. form an extra-chromosomal DNA molecule) as part of the same (or a single, or one, i.e. only one) DNA molecule. In such embodiments where the gene of interest and the origin of replication are excised as a single DNA molecule (or a single nucleic acid sequence), the gene of interest and the origin of replication may be located close to one another in the chromosome, for example such that it is readily possible for these components to be excised together, for example in a single excision event, e.g. a single recombination event.

The excising step and the forming step of the method of the invention may take place sequentially. For example, further modifications of the excised DNA molecule may be desired or needed after the excising step, before the extra-chromosomal DNA molecule is formed. For example, the one or more nucleic acid sequences excised during the excising step may be excised as one or more linear DNA molecules, in which case the method (e.g. the forming step of the method) may comprise ligating one or more ends of the excised DNA molecules together, for example to join (or ligate) multiple excised DNA molecules together (in embodiments where multiple DNA sequences are excised from the chromosome) and/or to circularise the excised DNA sequence(s). For example, where the “one or more nucleic acid sequences” as referred to herein consists of a single (or one, i.e. only one) DNA sequence, the method may comprise (or the forming step of the method may comprise) ligating the two ends of the excised DNA sequence together, e.g. to circularise the excised DNA sequence. In such an example, said circularisation (or circularisation event or circularisation reaction or circularisation process) may be considered to form the “extra-chromosomal DNA molecule” (e.g. a single extra-chromosomal DNA molecule) as referred to herein, and thus the excising step and forming step could be considered to be sequential. Circularisation of nucleic acid sequences is achievable through standard methods which are known in the art, for example using DNA ligase.

Of course, in embodiments where it is contemplated that the excising step and forming step are sequential, it is meant that the excising step takes place before the forming step.

Alternatively and more preferably, the excising step and the forming step of the method of the invention take place simultaneously.

For example, this may be the case if the excising step were to comprise the excision of the gene of interest together with (or as part of the same molecule as) an (or the) origin of replication, with no further modification to the excised DNA molecule thereafter. In such an example, the excised DNA molecule could be considered to be the extra-chromosomal DNA molecule, and thus the excising step and the forming step could be considered to be simultaneous.

In more preferred embodiments, the excising step and the forming step may be performed together, e.g. be a single step, event or reaction, e.g. be a single recombination step (or single recombination event or reaction). Thus in another aspect the invention provides a method of producing a product by a cell, the method comprising recombining a nucleic acid sequence from a chromosome in the cell to form an extra-chromosomal DNA molecule, wherein the nucleic acid sequence (or the extra-chromosomal DNA molecule) comprises a gene of interest and an origin of replication. Other aspects and embodiments of the invention described herein may be applied to this aspect of the invention mutatis mutandis where appropriate. Thus, for example, references to “excising and forming” would be framed in this context as “recombining”, and references to an “excised” nucleic acid sequence(s) would be framed in this context as a “recombined” nucleic acid sequence(s).

In embodiments, the invention provides a method of producing a product by a cell, the method comprising:
recombining a nucleic acid sequence from a chromosome in the cell to form an extra-chromosomal DNA molecule, wherein the nucleic acid sequence (or the extra-chromosomal DNA molecule) comprises a gene of interest and an origin of replication; and
inducing expression of the gene of interest from the extra-chromosomal DNA molecule.

In the present specification, the process of excising of the nucleic acid sequence(s) from the chromosome and the forming of the extra-chromosomal DNA molecule (in some preferred embodiments, the recombination step or event) may also be referred to as “POPing” or “POPing out”.

The excising and forming steps can be carried out using standard genome editing techniques in the art, for example using a CRISPR/Cas system or recombinases, for example using site-specific recombination, for example using a serine recombinase or tyrosine recombinase system.

In another aspect, the invention provides a method of producing a product by a cell, wherein the cell comprises a chromosome comprising a gene of interest and an origin of replication, the method comprising:
excising one or more nucleic acid sequences from the chromosome, wherein the one or more nucleic acid sequences comprises a gene of interest and an origin of replication; thereby forming (or in order to form) an extra-chromosomal DNA molecule, wherein the extra-chromosomal DNA molecule comprises (or consists of) the excised gene of interest and an (or the) excised origin of replication.

In embodiments, the invention provides a method of producing a product by a cell, wherein the cell comprises a chromosome comprising a gene of interest and an origin of replication, the method comprising:
excising one or more nucleic acid sequences from the chromosome, wherein the one or more nucleic acid sequences comprises a gene of interest and an origin of replication; thereby forming (or in order to form) an extra-chromosomal DNA molecule, wherein the extra-chromosomal DNA molecule comprises (or consists of) the excised gene of interest and an (or the) excised origin of replication; and
inducing expression of the gene of interest from the extra-chromosomal DNA molecule.

In another aspect, the invention provides a method of producing a product by a cell, the method comprising:
excising a gene of interest and an origin of replication from a chromosome in the cell; and
forming an extra-chromosomal DNA molecule in the cell comprising the excised gene of interest and an (or the) excised origin of replication.

In embodiments, the invention provides a method of producing a product by a cell, the method comprising:
excising a gene of interest and an origin of replication from a chromosome in the cell;
forming an extra-chromosomal DNA molecule in the cell comprising the excised gene of interest and an (or the) excised origin of replication; and
inducing expression of the gene of interest from the extra-chromosomal DNA molecule.

Other aspects and embodiments of the invention described herein may be applied to these aspects of the invention mutatis mutandis where appropriate.

The type(s) of origin of replication that is used in the extra-chromosomal DNA molecule is not particularly limited. For example, the origin of replication may be an inducible origin of replication, or a non-inducible (or active, or constantly active, or constitutive) origin of replication, or an origin of replication that is not inducible artificially or by artificial means.

In embodiments, the origin of replication (and/or the second origin of replication in the relevant embodiments as defined herein) is non-inducible; preferably a chromosomal origin of replication or oriC; more preferably Escherichia coli oriC.

In embodiments, the origin of replication (and/or the second origin of replication in the relevant embodiments as defined herein) is inducible; preferably a plasmid origin of replication or oriV; more preferably the origin of replication or oriV of plasmid RK2, pBR322, pMB1, ColE1, R6K, p15A, pSC101 or pUC.

In the art, in a large number of plasmids, oriV (or the “origin of vegetative replication”) often comprises or consists of direct repeats or iteron DNA sequences, which interact with Rep proteins to form the initial complex during the process of replication initiation.

In embodiments, the origin of replication may be stringent or relaxed.

In embodiments, the origin of replication (or activity of the origin of replication) is inducible by, or activatable by, or controllable by, or under the control of, an agent. The use of an inducible (or activatable or controllable etc.) origin of replication is advantageous because it enables the timing of replication of the extra-chromosomal DNA molecule and the level of replication (or copy-number) of the extra-chromosomal DNA molecule to be controlled (i.e. the extra-chromosomal DNA comprising the gene of interest and said origin of replication).

In embodiments, the origin of replication is an origin of replication which is induced or activated (or only induced or only activated) upon formation of the extra-chromosomal DNA molecule. Alternatively viewed, the origin of replication is inactive when in its initial location, e.g. on the chromosome (or before formation of the extra-chromosomal DNA molecule), but is active when part of the extra-chromosomal DNA molecule.

The origin of replication may be under the control of (or dependent upon the presence or absence or level of) an exogenous or heterologous agent (or inducer). The origin of replication may be induced by administration or inoculation of an agent (or inducer), for example by manual administration or inoculation of an agent (or inducer), for example by a human operator into the cell or cell population or cell culture. Alternatively, the origin of replication may be induced by formation of the extra-chromosomal DNA molecule.

The biochemical mechanism by which the origin of replication is rendered inducible (or repressible or controllable) is not particularly limited. For example, an agent (or inducer) which induces the activation of the origin of replication may be constitutively expressed or inducibly expressed. This may be achieved for example by operatively linking a gene encoding the agent to a promoter. The promoter may be an inducible promoter or a constitutive promoter. The promoter may be operatively linked to the agent by (or through or via or upon or as a result of) formation of the extra-chromosomal DNA molecule.

For example, the inducible origin of replication may be oriV (originally from RK2 plasmid) under the control of the protein TrfA. oriV requires TrfA for activation (i.e. for initiation of replication) and if TrfA is not present (or is not expressed), then oriV is inactive (i.e. does not initiate replication).

In embodiments, the origin of replication is an endogenous or homologous origin of replication, or a chromosomal origin of replication, or a native or wild-type chromosomal origin of replication, or an endogenous or homologous chromosomal origin of replication.

In embodiments, the origin of replication is a (or a naturally or a native) chromosomal (or chromosome derived, or chromosome originating, or naturally or native chromosomal) origin of replication. By this, it is meant that the origin of replication is naturally (or originally or endogenously or usually) located in a chromosome (irrespective of where it is located in a cell before, during or after the method of the invention is performed). For example, the origin of replication may be oriC, for example from E. coli. Such an origin of replication is thus typically native or endogenous to (or is the wild-type origin of replication of) the type of cell that is being used to produce a product in the methods of the invention.

In embodiments, the origin of replication is an (or a naturally) extra-chromosomal (or non-chromosomal) origin of replication (or extra-chromosomally derived, or extra-chromosomally originating, or naturally extra-chromosomal), preferably a plasmid derived or vector derived (or plasmid originating or vector originating, or naturally plasmid or vector derived) origin of replication (irrespective of where it is located in a cell before, during or after the method of the invention is performed). For example, the origin of replication may be oriV, for example from RK2 plasmid.

In embodiments, the origin of replication is an origin of replication whose normal or natural location is within a cell which is different to the cell being used in the method. For example, the origin of replication may be an exogenous or heterologous or non-native origin of replication, or an extra-chromosomal (or extra-chromosomally derived) origin of replication (e.g. a non-chromosomal origin of replication or an origin of replication from a plasmid or vector), or an exogenous or heterologous extrachromosomal (e.g. plasmid or vector) origin of replication. For example (and as already mentioned above), the origin of replication may be oriV, for example from RK2 plasmid. Thus a heterologous origin of replication is engineered into the chromosome.

In embodiments, the extra-chromosomal DNA molecule (i.e. the extra-chromosomal DNA molecule formed during the forming step of the method of the invention) comprises two or more origins of replication, for example two, three, four or five origins of replication, preferably two origins of replication. For example, the extra-chromosomal DNA molecule may comprise a chromosomal (or naturally chromosomal) origin of replication and an extra-chromosomal (or naturally extra-chromosomal or naturally non-chromosomal) origin of replication.

An example of a POP system designed by the present inventors is POP2 (an example of which is shown in FIG. 1). In the case of POP2, the chromosome, before the excising and forming steps (before POPing), contains two origins of replication (oriV and oriC). Then, in the POPing process, oriV is excised from the chromosome together with a gene of interest as a single unit to form a plasmid.

In embodiments, the origin of replication and the gene of interest which then form part of the extra-chromosomal DNA molecule are excised from the chromosome as part of the same nucleic acid sequence (or the same DNA molecule).

In embodiments, the nucleic acid sequence(s) (i.e. the nucleic acid sequence(s) to form the extra-chromosomal DNA molecule) are excised from the chromosome by site-specific recombination. Site-specific recombination involves the activity of site-specific recombinases at site-specific recombination sites. Thus in order to achieve excision by site-specific recombination, the gene of interest (and optionally, and preferably, the origin of replication) may be flanked by site-specific recombination sites. The cell may thus also comprise (or comprise one or more genes encoding) one or more site-specific recombinases which are directed to or specific for those site-specific recombination sites. Thus, in embodiments the nucleic acid sequence(s) is (are) flanked by site-specific recombination sites.

In embodiments, the excising step and the forming step take place simultaneously.

In embodiments, the excising step and the forming step take place simultaneously by site-specific recombination.

In embodiments, the origin of replication of the extra-chromosomal DNA molecule is an extra-chromosomal (i.e. naturally extra-chromosomal or non-chromosomal) origin of replication, or a non-native origin of replication, or an artificially inducible origin of replication, more preferably a plasmid origin of replication such as oriV, more preferably oriV from RK2 plasmid.

Where the origin of replication is inducible, the cell may comprise a gene (or a gene encoding an agent, or an agent) for controlling the activity (or induction) of the origin of replication. The gene may be located on the (or a) chromosome or may be located extra-chromosomally.

A gene for controlling activity of the origin of replication may be located chromosomally (or on the (or a) chromosome) or may be located extra-chromosomally (for example after the excising step or forming step, or throughout the method). Thus, in embodiments, the origin of replication (or activity of the origin of replication) on the extra-chromosomal DNA molecule is controlled by a gene which is located chromosomally (i.e. on the (or a) chromosome) or extra-chromosomally.

A gene for controlling activity of the origin of replication may be located chromosomally throughout the method, or may remain on the (or a) chromosome throughout the method. Thus, in embodiments, the origin of replication (or the activity of the origin of replication) is controlled by a gene which is located on the (or a) chromosome.

A gene for controlling activity of the (or a) origin of replication may be located extra-chromosomally throughout the method, or may remain outside the chromosome (or extra-chromosomal) throughout the method. Thus, in embodiments, the (or a) origin of replication (or the activity of the (or a) origin of replication) is controlled by a gene which is located outside the chromosome (or extra-chromosomally).

A gene for controlling activity of the origin of replication may be excised from the chromosome as part of the excising step. More preferably, said gene is excised as part of the same nucleic acid sequence as the gene of interest (and optionally, and preferably, the (or a) origin of replication).

In embodiments, the one or more nucleic acid sequence comprise or comprises (or is (or are) flanked by) site-specific recombination sites.

In embodiments, the nucleic acid sequence comprises a gene for controlling activity of the origin of replication.

The product production method of the invention may be further enhanced by combining it with the Switcher Technology first disclosed in Patent application WO2022117827A1. Specifically, this involves the inactivation of the origin(s) of replication in the chromosome, preferably by excision of the origin(s) of replication from the chromosome. In cells which contain only a single chromosome, the inactivation of the origin(s) of replication in the chromosome is advantageous because it stops cell division and growth while keeping the cells in a metabolically active state. The use of the Switcher Technology is also advantageous in the present invention because, by stopping cell division, the extent to which any copies of the extra-chromosomal DNA molecule may be lost from the cell is reduced.

Thus, in embodiments, the method of the invention involves inactivating an origin of replication in the chromosome. In embodiments, as a result of the method the chromosome lacks a functional origin of replication (or has no functional origin of replication, or does not have a functional origin of replication). This may be achieved by excising the endogenous chromosomal origin of replication from the chromosome, or by mutating the endogenous chromosomal origin of replication, for example so that it can no longer function. This is advantageous because it can result in an increase in protein production. Thus, in embodiments, the method comprises inactivating the endogenous (or native) chromosomal origin of replication (or origins of replication) in the chromosome, preferably wherein as a result of the method the chromosome lacks a functional origin of replication.

Thus, in embodiments of the method of the invention, as a result of the method, the chromosome lacks (or does not comprise or does not contain) a functional origin of replication.

Thus, in embodiments, the method of the invention comprises excising a second origin of replication from the chromosome, preferably wherein the extra-chromosomal DNA molecule comprises the second excised origin of replication.

In embodiments, the nucleic acid sequence(s) (to be excised from the chromosome) comprise a gene of interest and two origins of replication, and the extra-chromosomal DNA molecule comprises the gene of interest and one or both origins of replication.

In embodiments, the nucleic acid sequence(s) (to be excised from the chromosome) comprise a gene of interest and two origins of replication, and the extra-chromosomal DNA molecule comprises one of the origins of replication, but does not comprise the second origin of replication.

In embodiments, the endogenous (or native) chromosomal origin of replication (or origins of replication) is excised from the chromosome as part of the excising step (for example as part of the same nucleic acid sequence as the gene of interest), and said endogenous and/or chromosomal origin of replication forms part of the extra-chromosomal DNA molecule. Thus, in such embodiments the endogenous and/or chromosomal origin of replication is transferred from the chromosome to form part of the extra-chromosomal DNA molecule.

In embodiments, the one or more nucleic acid sequences (i.e. to be excised from the chromosome as described herein) comprises two origins of replication. In such embodiments, the one of the origins of replication (preferably a non-native or heterologous or artificially inducible or plasmid origin of replication) may be excised to form part of the extra-chromosomal DNA molecule, while the other origin of replication (preferably a native or chromosomal or endogenous origin of replication) does not form part of the extra-chromosomal DNA molecule. Alternatively, both origins of replication may form part of the extra-chromosomal DNA molecule.

Alternatively viewed, in embodiments, the method comprises irreversibly inactivating the origin of replication (or all of the origins of replication) in the chromosome (or the chromosomes) of the cell, or excising the origin of replication (or all of the origins of replication) from the chromosome (or the chromosomes) of the cell. For example, where the chromosome of the cell (as modified for the present invention) comprises one origin of replication (or two origins of replication), the method comprises excising the origin of replication (or both origins of replication) from the chromosome.

Combining the two technologies led to three different designs—POP1, POP3 (which includes POP3A and POP3B) and POP4—general schematics of which are shown in the Figures and are explained in more detail in the Examples. Thus, the POP2 system is unlike POP1, POP3 and POP4 in that in POP1, POP3 and POP4 the origin (endogenous origin) of replication (for example oriC) is removed from the chromosome as part of the method, whereas this is not done in the POP2 design. In other words, in the POP2 design, the native or endogenous or wild-type chromosomal origin(s) of replication remains in the chromosome, e.g. is not removed or irreversibly inactivated.

POP5, POP6 and POP7 were also designed. These are the same as POP1 but with the trfA gene and RK2 oriV replaced with pMB1-type origins. These were generated to confirm that the POP concept is not restricted to the plasmid types whose replication is Rep protein dependent.

POP8 was also designed. This is a chemical inducer-free system. In particular, no chemical inducer needs to be added in order to induce expression of the gene of interest from the plasmid, because expression of the gene of interest is induced by formation of the plasmid.

In the case of POP2, the chromosome, before POPing, contains two origins of replication (oriV and oriC). Then, in the POPing process, oriV is excised from the chromosome together with the gene of interest as a single unit to form a plasmid. Meanwhile the other origin of replication (oriC) is not excised and remains on the chromosome.

Thus, in embodiments, the one or more nucleic acid sequence(s) to be excised from the chromosome comprise a gene of interest and two origins of replication, and the extra-chromosomal DNA molecule comprises the excised gene of interest and an excised origin of replication (or one of, or at least one of, said excised origins of replication). More preferably, the extra-chromosomal DNA molecule comprises the excised gene of interest and one of the excised origins of replication, but does not comprise the other excised origin of replication. Preferably, the origins of replication consist of one non-native (or heterologous) or inducible (or artificially inducible) origin of replication (preferably a plasmid origin of replication, more preferably oriV, more preferably oriV from RK2 plasmid) and one native or wild-type or non-inducible (or not artificially inducible) origin of replication (preferably a chromosomal origin of replication, more preferably oriC, more preferably E. coli oriC). Preferably, the origin of replication that forms part of the extra-chromosomal DNA molecule is a non-native (or heterologous etc.) origin of replication and/or the origin of replication that does not form part of the extra-chromosomal DNA molecule is a native (or wild-type etc.) origin of replication.

In the case of POP1 and POP8, the chromosome, before POPing, contains two origins of replication (oriV and oriC). Then, in the POPing process, both origins of replication are excised from the chromosome together with gene of interest as a single unit to form a plasmid. POP1 and POP8 differ in that, in POP1, the gene of interest is under the control of an inducible promoter (both before and after POPing), whereas, in POP8, the gene of interest is operatively linked with a constitutive promoter through POPing (i.e. by formation of the plasmid).

Thus, in embodiments, the one or more nucleic acid sequence(s) to be excised from the chromosome comprise a gene of interest and two origins of replication, and the extra-chromosomal DNA molecule comprises the excised gene of interest and the two excised origins of replication. Preferably, the origins of replication consist of one non-native (or heterologous) or inducible (or artificially inducible) origin of replication (preferably a plasmid origin of replication, more preferably oriV, more preferably oriV from RK2 plasmid) and one native or wild-type or non-inducible (or not artificially inducible) origin of replication (preferably a chromosomal (or native chromosomal or wild-type chromosomal) origin of replication, more preferably oriC, more preferably E. coli oriC).

In the case of POP5, POP6 and POP7, the chromosome, before POPing, contains two origins of replication (oriC and the pMB1-type origin). Then, in the POPing process, both origins of replication are excised from the chromosome together with the gene of interest as a single unit to form a plasmid.

In the case of POP4, the chromosome, before POPing, contains one origin of replication (oriC). Then, in the POPing process, the origin of replication is excised from the chromosome together with the gene of interest as a single unit to form a plasmid.

Thus, in embodiments, the one or more nucleic acid sequence(s) (to be excised from the chromosome) comprise the gene of interest and an origin of replication, and the extra-chromosomal DNA molecule comprises the excised gene of interest and an (or the) excised origin of replication. Preferably, the origin of replication (or the excised origin of replication) is a native or non-inducible (or not artificially inducible) origin of replication (preferably a chromosomal (or native chromosomal or wild-type chromosomal) origin of replication, more preferably oriC, more preferably E. coli oriC). Alternatively, the origin of replication is a non-native (or heterologous) or inducible (or artificially inducible) origin of replication (preferably a plasmid origin of replication, more preferably oriV, more preferably oriV from RK2 plasmid).

In the case of POP3A and POP3B, the chromosome, before POPing, contains two origins of replication (oriV and oriC). Then, in the POPing process, oriV is excised from the chromosome together with the gene of interest as a single unit to form a plasmid. Additionally, oriC is excised as a separate molecule, which does not form part of the plasmid (extra-chromosomal DNA molecule) comprising the excised gene of interest. Hence, in POP3 the excision of oriC and the POP plasmid are two separate events.

Thus, in embodiments of the method of the invention, the one or more nucleic acid sequence(s) (to be excised from the chromosome) comprise the gene of interest and an origin of replication, and the method comprises a step of irreversibly inactivating all of the origins of replication in the chromosome (or all the chromosomes in the cell). Preferably, the origin of replication is a native or non-inducible (or not artificially inducible) origin of replication (preferably a chromosomal (or native chromosomal or wild-type chromosomal) origin of replication, more preferably oriC, more preferably E. coli oriC). Alternatively, the origin of replication is a non-native (or heterologous) or inducible (or artificially inducible) origin of replication (preferably a plasmid origin of replication, more preferably oriV, more preferably oriV from RK2 plasmid).

Alternatively, in embodiments of the method of the invention, the one or more nucleic acid sequence(s) (to be excised from the chromosome) comprise the gene of interest and two origins of replication, and the extra-chromosomal DNA molecule comprises the gene of interest and an origin of replication (or one of, or at least one of, the origins of replication).

Alternatively, in embodiments of the method of the invention, the one or more nucleic acid sequence(s) (to be excised from the chromosome) comprise the gene of interest and two origins of replication, and the extra-chromosomal DNA molecule comprises the gene of interest and one of said origins of replication but does not comprise the other origin of replication.

An advantage of the present invention is that expression of the gene of interest can take place in the cell at high copy-number without the need for a selectable marker (a marker for selection) within the extra-chromosomal DNA molecule. Without wishing to be bound by theory, it is understood that no selection or antibiotic resistance gene is necessary because formation of the extra-chromosomal DNA molecule takes place late in the growth stage and there are not enough cell divisions left to lose the extra-chromosomal DNA molecule from the cells. This reduction in the loss of extra-chromosomal DNA molecules from the cells may also be achieved or further improved where the Switcher technology is used, i.e. when the method (e.g. the excising and forming steps) results in the inactivation of the chromosomal origin of replication, such that the cell no longer comprises a functional origin of replication in its chromosome(s).

Thus, in preferred methods of the invention, selectable markers such as antibiotic resistance genes are not required or are not used.

A marker for selection is typically a nucleotide sequence encoding a protein that confers selective resistance upon a cell in which it is expressed. The marker for selection is typically an antibiotic resistance gene. The chloramphenicol resistance gene, the ampicillin resistance gene and the kanamycin resistance gene are typical selection markers. Other markers for selection include a gene conferring the ability to use an artificial nitrogen source or gene conferring the ability to use an artificial carbon source. Thus, in embodiments the nucleic acid sequence(s) to be excised from the chromosome and/or the extra-chromosomal DNA molecule do not comprise (or contain or have) these components.

Thus, in embodiments, the one or more nucleic acid sequence(s) does not comprise a selectable marker.

Thus, in embodiments the nucleic acid sequence(s) to be excised from the chromosome, and/or the extra-chromosomal DNA molecule, do not comprise (or do not contain, or do not have) a marker for selection (or do not comprise (or do not contain, or do not have) a selection or a selectable marker.

In embodiments, the nucleic acid sequence(s) to be excised from the chromosome, and/or the extra-chromosomal DNA molecule, do not comprise (or do not contain, or do not have) a marker for selection wherein the marker for selection is a nucleotide sequence encoding a protein that confers selective resistance upon a cell in which it is expressed.

In embodiments, the nucleic acid sequence(s) to be excised from the chromosome, and/or the extra-chromosomal DNA molecule, do not comprise (or do not contain, or do not have) an antibiotic resistance gene.

In embodiments, the nucleic acid sequence(s) to be excised from the chromosome, and/or the extra-chromosomal DNA molecule, do not comprise (or do not contain, or do not have) the chloramphenicol resistance gene, the ampicillin resistance gene and/or the kanamycin resistance gene.

In embodiments, the nucleic acid sequence(s) to be excised from the chromosome, and/or the extra-chromosomal DNA molecule, does not comprise (or do not contain, or do not have) a gene conferring the ability to use an artificial nutrient source.

In embodiments, the nucleic acid sequence(s) to be excised from the chromosome, and/or the extra-chromosomal DNA molecule, do not comprise (or do not contain, or do not have) a gene conferring the ability to use an artificial nitrogen source and/or a gene conferring the ability to use an artificial carbon source.

In embodiments, the nucleic acid sequence(s) to be excised from the chromosome, and/or the extra-chromosomal DNA molecule, do not comprise (or do not contain, or do not have) a dnaA gene, or do not comprise (or do not contain, or do not have) a dnaA gene from Bacillus subtilis or do not comprise (or do not contain, or do not have) a Bacillus subtilis origin of replication.

In embodiments, the nucleic acid sequence(s) to be excised from the chromosome, and/or the extra-chromosomal DNA molecule, do not comprise (or do not contain, or do not have) a toxin, anti-toxin, and/or auxotrophic marker.

Thus, an exemplary cell may, before the excising and forming steps are performed, comprise a chromosome comprising a gene of interest and an origin of replication flanked by recombination sites, e.g. site-specific recombination sites.

In embodiments, the one or more nucleic acid sequences as defined herein (i.e. the one or more nucleic acid sequences to be excised from the chromosome) is or are (or are each) flanked by site-specific recombination sites (e.g. site-specific recognition and cleavage sites) for recognition (or recognition and cleavage) by a site-specific recombinase. In the case of multiple nucleic acid sequences, the pairs of site-specific recombination sites may be the same or different. In the case of multiple nucleic acid sequences, the pairs of site-specific recombination sites may be appropriately complementary so that, following excision, the multiple excised nucleic acid sequences may be joined (or ligated) to form the extra-chromosomal DNA molecule as described herein.

A relevant site-specific recombinase can then bind and cleave said site so as to facilitate or enable DNA recombination.

In embodiments, the one or more nucleic acid sequences (to be excised from the chromosome) is or are (or are each) flanked by site-specific recombination sites, wherein each site is recognizable by a site-specific recombinase enzyme or enzymes. For example, where one nucleic acid sequence is to be excised in the excising step of the invention, two site-specific recombination sites may be present, one positioned 5′ to (or at the 5′ end of, or at one end of) the nucleic acid sequence, and one positioned 3′ to (or at the 3′ end of, or at the other end of) the nucleic acid sequence. Where multiple nucleic acid sequences are to be excised in the excising step of the invention, multiple pairs of site-specific recombination sites may be present, for example one for each nucleic acid sequence, for example wherein each pair flanks each nucleic acid sequence.

In embodiments, the site-specific recombination sites (or the sites within each pair of site-specific recombination sites) have different sequences, for example attB and attP sites. Alternatively, the site-specific recombination sites (or the sites within each pair of site-specific recombination sites) have the same sequence (i.e. the site-specific recombination sites are identical), for example loxP sites. The appropriate site-specific recombination sites are chosen depending on the recombinase enzyme being used and would be readily derivable for a person skilled in the art.

The term “recombinase” as used herein encompasses any site-specific enzyme capable of the excising and/or forming steps of the invention. Such recombinases may be capable of DNA manipulation by excision, insertion, inversion or translocation. The term “recombinase” encompasses not only recombinases but also integrases, invertases and resolvases.

The term “site-specific recombinase” (or site-specific integrase or site-specific invertase or site-specific resolvase) as used herein means an enzyme which catalyzes DNA exchange reactions between target site sequences that are specific to that recombinase. Hence, the site-specific recombinases (and site-specific recombinase sites) as used herein can facilitate the excising and/or forming steps of the invention.

The relative orientation of site-specific recombination sites, e.g. a pair of site-specific recombination sites, determines the outcome of the recombination event. The term “orientation” (or direction or directionality) of the site-specific recombination sites as used herein is generally understood in the art and relates to the direction that the site-specific recombinase site is provided in the polynucleotide. For example, where site-specific recombination sites, e.g. a pair of site-specific recombination sites, on the same polynucleotide have opposite orientations, inversion of the flanked sequence within the polynucleotide may occur (for example with a pair of loxP sites (in the Cre-lox system), or with a pair consisting of an attP site and an attB site (for example as found in serine recombinase systems). In contrast, where two site-specific recombination sites have the same orientation (or parallel orientation), excision of the flanked sequence from the polynucleotide may occur.

Hence, in embodiments the site-specific recombination sites (preferably serine site-specific recombination sites) flank (i.e. are positioned either side of, or at either end of) the nucleic acid sequence in the chromosome (i.e. the nucleic acid sequence to be excised) in an orientation such that site-specific recombination (e.g. when catalysed by an appropriate enzyme) leads to excision of the nucleic acid sequence from the chromosome (and optionally also the formation, or simultaneous formation, of the extra-chromosomal DNA molecule). Preferably, the site-specific recombination sites are positioned in the same orientation in the chromosome.

In embodiments, the site-specific recombination sites comprise site-specific recombination sites for recognition by a serine recombinase. Preferably, the serine recombinase is γδ, Bxb1, φC31 or TP901. However, there are many different serine recombinases described in the art and experimentally and more than 4000 new ones predicted by bioinformatics (Yang et al., 2014) [12]. Any of these may be used.

In embodiments, the site-specific recombination sites comprise site-specific recombination sites for recognition by a serine integrase.

The mechanism of recombination by serine site-specific integrases (a subfamily of serine site-specific recombinases) is generally understood in the art. Serine site-specific integrases catalyse recombination of attP and attB sites, generating attL and attR sites. The outcome of recombination of attP and attB sites is dependent on the orientation of the site-specific recombination sites. Recombination of an attP site and an attB site having the same orientation and located on the same polynucleotide results in excision of the flanked sequence from the polynucleotide, leaving an attR or attL site in the remaining polynucleotide.

In embodiments, the site-specific recombination sites comprise site-specific recombination sites for recognition by a tyrosine recombinase, preferably wherein the tyrosine recombinase is Cre, Dre, Flp, KD, B2 or B3.

Examples of site-specific recombinase technologies suitable for use in the present invention include serine site-specific recombination, tyrosine site-specific recombination, Cre-Lox recombination, FLP-FRT recombination, and homologous recombination.

In embodiments, the site-specific recombination sites comprise or consist of a pair of site-specific recombination sites.

The term “pair” of sites as used herein refers to two sites which are recognised by the same site-specific recombinase enzyme and wherein both sites must be present for the recombination event to occur. Hence, an example of a pair of site-specific recombination sites is an attP site and an attB site (e.g. for recognition by a serine recombinase/integrase), or two loxP sites (e.g. for recognition by Cre recombinase).

Other examples of pairs of site-specific recombination sites would be known in the art. Hence, other examples of pairs of site-specific recombination sites are: two FRT sites (e.g. for recognition by Flp recombinase); two rox sites (e.g. for recognition by Dre recombinase); two KD recombinase target (KDRT) sites (e.g. for recognition by KD recombinase); two B2 recombinase target (B2RT) sites (for recognition by B2 recombinase); or two B3 recombinase target (B3RT) sites (for recognition by B3 recombinase).

Thus, a “pair” of sites as described herein can be two different sites (with two different sequences) or two identical sites (with the same sequences) depending on the recombinase enzyme being used.

It is possible that a modified cell, comprising nucleic acid sequences to be excised from the chromosome in accordance with the invention, could undergo changes which inadvertently render the nucleic acid sequences non-excisable. Loss of excisability may be caused, for example, by mutation of one or more site-specific recombination sites, or by mutation of one or more genes encoding the site-specific recombinase(s) specific for said sites.

Hence, it may be beneficial to provide a cell wherein the nucleic acid sequence (or, in case of multiple nucleic acid sequences, each nucleic acid sequence) to be excised from the chromosome is flanked by two, three or four (or at least, or up to, two, three, or four) pairs of site-specific recombination sites, preferably two pairs of site-specific recombination sites. Without wishing to be bound by theory, the probability of such cells losing the ability to excise the nucleic acid sequence from the chromosome is reduced as compared to cells wherein the nucleic acid sequence is flanked by or comprises only one pair of site-specific recombination sites.

Hence, in embodiments, the site-specific recombination sites comprise or consist of two or more pairs, preferably two pairs, of site-specific recombination sites.

Alternatively viewed, the site-specific recombination sites comprise or consist of three, four, five or six pairs of site-specific recombination sites.

Where there are two or more pairs of site-specific recombination sites flanking the (or each) nucleic acid sequence to be excised from the chromosome, the pairs of site-specific recombination sites may be provided in any order along the chromosome providing the order is such so as to allow successful recombination to occur.

In embodiments, where two pairs of site-specific recombination sites are present, one pair of site-specific recombination sites can be nested within the other, i.e. one pair of recombination sites is flanked by the other pair of recombination sites. Hence, for two pairs of recombination sites A and B, the sites are arranged along the polynucleotide in the order A-B-B-A, or in the order B-A-A-B.

Alternatively, the site-specific recombination sites from each pair are provided alternately along the polynucleotide, such that neither pair is nested within the other pair; in other words, for two pairs of recombination sites A and B, the sites are arranged in the polynucleotide in the order A-B-A-B, or in the order B-A-B-A.

The individual components of the two or more pairs of recombination sites are spatially arranged in such a way that recombination can occur. Thus, in a preferred embodiment, an appropriate number of nucleotides separate the adjacent A and B sites (i.e. the sites from each pair which are adjacent). In an alternative preferred embodiment, no nucleotides separate the adjacent A and B sites (i.e. the adjacent sites from each pair); in other words, the adjacent A and B sites (i.e. the adjacent sites from each pair) form one contiguous sequence.

The embodiments provided in the above paragraphs apply to further sets of pairs of site-specific recombination sites, for example three site-specific recombination sites, mutatis mutandis.

In embodiments, the cell of the invention further comprises a gene encoding a site-specific recombinase.

The excising and forming step can be facilitated by site-specific recombinases (or site-specific recombinase activity). This can be achieved by using a cell wherein the chromosome has been modified such that the nucleic acid sequences to be excised are flanked by site-specific recombination sites. However, the method of excision is not particularly limited thereto; other genome editing techniques may be used in order to excise the nucleic acid sequences described herein, for example using tyrosine recombinases or CRISPR/Cas technology.

In embodiments, the gene encoding the site-specific recombinase is operatively linked to a promoter. For example, in the POP plasmids exemplified in the examples (POP1-8), the site-specific recombinases are φC31 integrase and Bxb1 under the control of mutated λPR(T41C) promoter and λPL promoter respectively.

The site-specific recombinase is selected appropriately such that it can act with the chosen site-specific recombination sites. Thus, in embodiments where two or more pairs of recombination sites are used, the cell (or the chromosome within the cell) may further comprise a gene encoding a site-specific recombinase for each of the pairs of recombination sites, as appropriate, e.g. genes encoding 2 or more recombinases, e.g. 3, 4, 5 or 6 recombinases, as appropriate. In addition, the genes encoding each of the site-specific recombinase enzymes may be operatively linked to the same or different promoters. In addition, the genes encoding each of the site-specific recombinase enzymes may be on the same or different nucleic acid sequences, e.g. on the same or different chromosome, or same or different extra-chromosomal DNA molecule, e.g. plasmid or construct; or one or more of said genes may be on a chromosome while others may be on an extra-chromosomal DNA molecule. Preferably said genes are located on a chromosome (or the same chromosome).

It will be appreciated that the embodiment of the promoters and induction systems described herein (for example in relation to the site-specific recombinases) may be alternatively or additionally be used to characterize the control of induction (or expression) of the gene of interest as described herein, mutatis mutandis.

The term “promoter” as used herein takes its art recognised meaning and is generally understood to mean a sequence of DNA to which proteins bind in order to control, initiate and/or inhibit transcription of the DNA sequence downstream of the promoter. Where it is stated that the gene encoding the recombinase enzyme (or gene of interest) is “operatively linked” to a promoter, it is meant that the promoter is in a correct functional location and/or orientation in relation to the gene to control transcription of the gene.

In embodiments, the promoter (for example the promoter for inducing the excising and/or forming steps, and/or the promoter of the gene of interest, and/or the promoter of the site-specific recombinase(s), and/or the promoter of an agent controlling the origin of replication) is sensitive to internal stimuli or internal conditions. For example, the promoter may be induced when the cell culture enters a specific phase of growth, as in case of PCP_2836 promoter (Ma et al., 2018, Microbial Cell Factories. BioMed Central Ltd., 17 (1), p. 185. doi: 10.1186/s12934-018-1031-7.), phoPR promoter (Paul et al., 2004, Journal of Bacteriology. American Society for Microbiology Journals, 186 (13), pp. 4262-4275. doi: 10.1128/JB.186.13.4262-4275.2004) and P170 promoter (Madsen et al., 1999, Molecular Microbiology. Blackwell Publishing Ltd., 32 (1), pp. 75-87. doi: 10.1046/j.1365-2958.1999.01326.x). Preferably, the promoter may be induced when the cell culture enters the exponential growth phase, or during the exponential growth phase. Other preferred settings include the use of auto-induction media (for example as described in Studier et al. 2005, Protein Expression and Purification, 2005, 41:207-234) and self-inducing systems (for example SILEX, as described in Briand et al. 2016, Scientific Reports, 6:33037).

Promoters (for example the promoter for inducing the excising and/or forming steps, and/or the promoter of the gene of interest, and/or the promoter of the site-specific recombinase(s), and/or the promoter of an agent controlling the origin of replication) which are sensitive to internal conditions are sometimes preferred in industrial settings since these promoters do not require intervention from the user (e.g. the administration of a chemical inducer) to be induced.

In embodiments, the promoter (for example the promoter for inducing the excising and/or forming steps, and/or the promoter of the gene of interest, and/or the promoter of the site-specific recombinase(s), and/or the promoter of an agent controlling the origin of replication) is sensitive to one or more external stimuli, for example is an inducible promoter. Hence, the promoter used can be selected based on the external stimulus or stimuli which the user wishes to initiate excising and/or forming with.

In some such embodiments, the promoter (for example the promoter for inducing the excising and/or forming steps, and/or the promoter of the gene of interest, and/or the promoter of the site-specific recombinase(s), and/or the promoter of an agent controlling the origin of replication) is temperature-sensitive, pH-sensitive, light-sensitive, or chemically-sensitive. Promoters with these and other properties, e.g. other inducible properties, are well known and described in the art and appropriate promoters for use in the invention (for example the promoter for inducing the excising and/or forming steps, and/or the promoter of the gene of interest, and/or the promoter of the site-specific recombinase(s), and/or the promoter of an agent controlling the origin of replication) could readily be selected.

For example, other types of gene expression control methods which can be used for the same purpose (for example for inducing the excising and/or forming steps, and/or for inducing expression of the gene of interest, and/or for inducing expression of the site-specific recombinase(s), and/or inducing expression of an agent controlling the origin of replication), include but are not limited to, induction by inducers (IPTG, arabinose, homoserine lactones, anhydrotetracycline etc) (Marschall, Sagmeister and Herwig, 2017)[16] (Lutz and Bujard, 1997)[15], (Cox, Surette and Elowitz, 2007)[17], targeted proteolysis (Cameron and Collins, 2014)[18], different variants of CRISPR/Cas9 (Qi et al., 2013)[19] etc. A person skilled in the art would know how to use such other types of gene expression control methods. For example, in the case of chemically induced promoters an appropriate chemical is added to the growth medium where it enters the cells either through diffusion or facilitated transport. Inside the cell it binds to its target transcriptional regulator and this leads to activation of transcription from the target promoters of said transcriptional regulator. If a transcriptional repressor controls the expression from the promoter and said transcriptional repressor is made sensitive to proteolysis then the expression from the promoter can be induced by inducing proteolysis of the said transcriptional regulator. Catalytically inactive CRISPR/Cas9 can be targeted to a promoter by expressing the appropriate guide RNA in the cell, and, depending on the exact configuration this can lead to either activation or repression of the said promoter.

Thus, in embodiments, the promoter (for example the promoter for inducing the excising and/or forming steps, and/or the promoter of the gene of interest, and/or the promoter of the site-specific recombinase(s), and/or the promoter of an agent controlling the origin of replication) is chemically-sensitive and suitable promoters would be readily available in the art. For example, preferred promoters are those wherein the promoter is inducible using isopropyl β-d-1-thiogalactopyranoside (IPTG), arabinose, homoserine lactones, or anhydrotetracycline, e.g. wherein the promoter is a promoter from the lac operon, ara operon, tet operon or lux operon, e.g. wherein the promoter is lac promoter (lac p), araBAD promoter, ppuI promoter, tet promoter (Pₜₑₜ promoter), or luxI promoter.

For example, the expression of a gene encoding a site-specific recombinase (and/or the expression of the gene of interest, and/or the expression of the site-specific recombinase(s), and/or the expression of an agent controlling the origin of replication) may be controlled under a lac operon expression system. The expression of said gene (and/or gene of interest) can be induced by IPTG or allolactose. For example, the gene (and/or gene of interest) may be provided on a polynucleotide sequence or construct (e.g. on a chromosome or on an extra-chromosomal DNA molecule or a plasmid) wherein the lac promoter and lac operator are provided upstream of the gene and wherein the polynucleotide sequence further comprises the lac repressor (lacI). Polynucleotide sequences and vectors suitable for control of target genes under this system are known in the art.

Alternatively, the expression of a gene encoding a site-specific recombinase (and/or the expression of the gene of interest, and/or the expression of the site-specific recombinase(s), and/or the expression of an agent controlling the origin of replication) may be controlled under an ara operon expression system. The expression of said gene (and/or gene of interest) can be induced by arabinose. For example, the gene (and/or gene of interest) may be provided on a polynucleotide sequence or construct (e.g. on a chromosome or on an extra-chromosomal DNA molecule), wherein the araBAD promoter is upstream of the gene (and/or gene of interest), and wherein the polynucleotide sequence or construct also comprises the other genetic elements necessary for gene regulation using the ara operon. The genetic elements of the ara operon necessary for gene regulation are known to the skilled person and are provided above.

Alternatively, and more preferably, the expression of a gene encoding a site-specific recombinase (and/or the expression of the gene of interest, and/or the expression of the site-specific recombinase(s), and/or the expression of an agent controlling the origin of replication) may be controlled under a lux operon expression system. The expression of said gene (and/or gene of interest) may be induced by acyl-homoserine lactones (or an acyl-homoserine lactone). For example, the gene (and/or gene of interest) may be provided on a polynucleotide sequence or construct (e.g. on a chromosome or on an extra-chromosomal DNA molecule), wherein the luxI promoter is upstream of (or operatively linked to) the gene, and wherein the polynucleotide sequence or construct also comprises the luxR gene, preferably wherein the luxR gene is constitutively expressed.

Alternatively, the cell may comprise a gene encoding a site-specific recombinase and the expression of said gene can be controlled under a Tet-Off or Tet-On expression system. The expression of said gene can be induced by tetracycline (or anhydrotetracycline or doxycycline). For example, the cell comprises (i) the gene provided on a plasmid, wherein a tetracycline-(or anhydrotetracycline- or doxycycline-) dependent promoter is upstream of the gene, and (ii) a tTA expression plasmid or an rtTA expression plasmid. Plasmids suitable for control of target genes under this system are known in the art.

In embodiments, expression of the gene encoding the site-specific recombinase (and/or the expression of the gene of interest) may be induced or controlled by targeted proteolysis.

In embodiments, expression of the gene encoding the site-specific recombinase (and/or the expression of the gene of interest) is induced or controlled by CRISPR interference (CRISPRi).

In preferred embodiments, the promoter (for example the promoter for inducing the excising and/or forming steps, and/or the promoter of the gene of interest, and/or the promoter of the site-specific recombinase(s), and/or the promoter of an agent controlling the origin of replication) is temperature-sensitive.

Any appropriate temperature-sensitive promoter can be used, of which there are many examples in the art.

A temperature-sensitive promoter used in the present invention can be selected to be activated (and/or at its highest activity level) at any suitable temperature. Preferably, the promoter may be a temperature-sensitive promoter which is activated (and/or at its highest activity level) at between 20 to 25° C., between 25 to 30° C., between 30 to 35° C., between 35 to 40° C., between 40 to 45° C., between 45 to 50° C., between 50 to 55° C., or between 55 to 60° C., or above 60° C. Preferably, the promoter may be activated (and/or at its highest activity level) at about 37° C.

Hence, in embodiments, a temperature-sensitive promoter used in the present invention (for example a promoter for inducing the excising and/or forming steps, and/or a promoter of the gene of interest) can be induced by changing (i.e. raising or lowering) the temperature of the cell (or cell culture) to the temperature at (or temperature range within) which the temperature-sensitive promoter is activated (and/or at its highest activity level). Hence, in order to induce the excising of the nucleic acid sequence(s) from the chromosome and the forming of the extra-chromosomal DNA molecule (and/or expression of the gene of interest, as the case may be), the temperature may be changed to between 20 to 25° C., between 25 to 30° C., between 30 to 35° C., between 35 to 40° C., between 40 to 45° C., between 45 to 50° C., between 50 to 55° C., or between 55 to 60° C., or above 60° C.

The temperature-sensitive promoter used may be activated at higher temperatures, in which case the temperature of the cell (or cell culture) may need to initially be lower (e.g. 30° C. or lower than 30° C.) and then raised (e.g. to 37° C., or higher than 37° C.) in order to induce activation and hence the excising of the nucleic acid sequence(s) from the chromosome and the forming of the extra-chromosomal DNA molecule (and/or expression of the gene of interest, as the case may be). Conversely, the temperature-sensitive promoter used may be activated at lower temperatures, in which case the temperature of the cell (or cell culture) may need to initially be higher (e.g. 37° C., or higher than 37° C.) and then lowered (e.g. to 30° C., or lower than 30° C.) in order to induce excising and forming (and/or expression of the gene of interest, as the case may be). Preferably, the promoter may be activated (and/or at its highest activity level) at or about 37° C. (or higher than 37° C.). Thus, conveniently the temperature of the cell (or cell culture) may initially be lower, e.g. at or about 30° C., e.g. between 25° C. and 33° C., and then raised (e.g. to at or about 37° C., e.g. between 36° C. and 39° C., or higher than 37° C.) in order to induce the excising and/or forming steps of the invention (and/or expression of the gene of interest, as the case may be).

After the temperature of the cell (or cell culture) has been raised or lowered so as to activate the temperature-sensitive promoter, the temperature may be further maintained, or may be altered (or lowered or raised) for example by the human operator, e.g. to provide an ideal or optimal temperature for expression of the gene of interest if this is different to the activation temperature (or activation temperature range) of the temperature-sensitive promoter. It is not a pre-requisite of the invention for the activation temperature to be maintained after the temperature-sensitive promoter has been activated. Thus, after the temperature of the cell (or cell culture) has been raised (or lowered) to match or exceed (or go below) the activation temperature of the temperature-sensitive promoter, the temperature may be lowered back down (or raised back up), for example to the previous temperature or starting temperature.

A preferred temperature-sensitive promoter for use in the present invention is a promoter which is controllable or regulated by a temperature-sensitive agent (or by a temperature-sensitive repressor, or by a temperature-sensitive activator), for example the phage lambda cI857 repressor. In embodiments, the temperature-sensitive promoter is phage lambda P_{R} or P_{L} promoter (or a modified version of P_{R} or P_{L} promoter), controlled by the lambda cI857 repressor, which are activated (and/or have their highest activity level) at or about 37° C. (Jechlinger et al., 1999, FEMS Microbiology Letters. Wiley/Blackwell (10.1111), 173 (2), pp. 347-352. doi: 10.1111/j.1574-6968.1999.tb13524.x). Thus, use of this promoter conveniently allows induction of the excising and forming steps of the invention (and/or expression of the gene of interest, as the case may be) by changing the temperature of the cell (or cell culture) from at or about 30° C. (e.g. between 25° C. and 33° C.), to at or about 37° C. (e.g. between 36° C. and 39° C., or higher than 37° C.).

In embodiments, functional elements, e.g. one or more or all of the functional elements (i.e. genetic elements) used in (or necessary for) the excising and/or forming steps of the invention are located on (or integrated in) one or more episomes or plasmids, or on some other appropriate extra-chromosomal DNA molecule within the cell. For example, in embodiments the genetic element(s) (or gene(s)) encoding the site-specific recombinase(s) is located on (or integrated in) one or more episomes or plasmids, or on one or more other appropriate extra-chromosomal elements within the cell. Alternatively, the entire recombinase expression system may be located on (or integrated in) one or more episomes or plasmids (or other appropriate elements). For example, the genetic element(s) (or gene(s)) encoding the site-specific recombinase(s), and the genetic elements (or genes) encoding the recombinase regulator components, may be located on (or integrated in) one or more episomes or plasmids (or other appropriate elements). The recombinase regulator components may include a temperature-sensitive promoter as defined herein (for example a P_{R} or P_{L} promoter or a modified version thereof), and optionally where appropriate a genetic element (or gene) encoding an agent which facilitates that sensitivity as defined herein, for example a gene encoding a temperature-sensitive repressor, for example a gene encoding the cI857 repressor.

In alternative and more preferred embodiments, functional elements, e.g. one or more or all of the functional elements (i.e. genetic elements) used in (or necessary for) the excising and/or forming steps of the invention are located on (or integrated in) the genome, for example on a chromosome (e.g. a bacterial chromosome). For example, in embodiments the genetic element(s) (or gene(s)) encoding the site-specific recombinase(s) is located on (or integrated in) a genome, for example on a chromosome (e.g. a bacterial chromosome). Alternatively and more preferably, the entire recombinase expression system may be located on (or integrated in) the genome. For example, the genetic elements (or gene(s)) encoding the site-specific recombinase(s), and the genetic elements (or gene(s)) encoding the recombinase regulator components, may be located on (or integrated in) the genome, for example on a chromosome. The recombinase regulator components may include a temperature-sensitive promoter as defined herein (for example a P_{R} or P_{L} promoter or a modified version thereof), and optionally where appropriate a genetic element (or gene) encoding an agent which facilitates that sensitivity as defined herein, for example a gene encoding a temperature-sensitive repressor, for example a gene encoding the cI857 repressor.

Alternatively, the genetic element(s) (or gene(s)) encoding the site-specific recombinase(s) may be located on the genome, while the genetic elements encoding the associated recombinase regulator components may be located on one or more episomes or plasmids; or vice versa.

In embodiments, one or more of the functional elements (i.e. genetic elements) used in (or necessary for) the irreversible inactivation of the origin of replication are codon optimized. For example, the genetic element(s) (or gene(s)) encoding the site-specific recombinase(s) may be codon optimized. Alternatively or in addition, the genetic element (or gene) encoding the temperature-sensitive agent which facilitates the sensitivity of the temperature-sensitive promoter (e.g. encoding a temperature-sensitive repressor, for example encoding cI857), is codon-optimized, where such temperature-sensitive promoter system is used.

In embodiments, the product is a product (or any product or any product of interest) produced in or producible by a cell, for example produced in or producible by a two-stage bioprocess method of the art. Such products are thus products which it is desired to prepare or produce in elevated, measurable or significant quantities, e.g. are desirable, useful, industrial, or commercial products, or products of value, e.g. to industry or research. Hence, in embodiments, the product is a small molecule. Alternatively or in addition, the product may be a polypeptide or protein or protein complex (or protein of interest), for example an antibody, antigen, enzyme, growth factor or cytokine. Preferably, the product is a pharmaceutical compound, medicinal product, food additive, colouring compound, fragrance component, industrial chemical, fine chemical or biofuel. Preferably, the biofuel is alcohol, for example butanol or isobutanol. Preferably, the product is encoded by the gene of interest, or the gene of interest encodes an enzyme or agent for producing the product.

The method of the invention is not limited to formation of only a single extra-chromosomal DNA molecule within the cell. For example, the method of the invention may comprise multiple (e.g. more than one) genes of interest and multiple origins of replication for the excision and formation of multiple extra-chromosomal DNA molecules (e.g. multiple plasmids) for the expression of said multiple genes of interest. This would be achievable for example by using separate excision and formation machinery for the excision and formation of each extra-chromosomal DNA molecule, e.g. using different serine recombinases (and using a cell comprising a chromosome with different corresponding site-specific recombination sites) for each nucleic acid sequence (or combination of nucleic acid sequences) required for the formation of each extra-chromosomal DNA molecule.

Steps necessary to enable a cell to produce a desired product, e.g. inducing or allowing expression of genes necessary for product synthesis or regulation or expression of the gene of interest, would be well known to a person skilled in the art. For example, where the product is a small molecule, one or more enzymes may be expressed which can produce the product from a given substrate. One or more of such enzymes may be encoded by the gene of interest(s) as described herein. The substrate can be available homogenously or endogenously in the cell, for example where the substrate is an intermediate of a metabolic pathway within the cell. Alternatively or in addition, said substrate may be provided heterologously (e.g. by expression of a heterologous gene in a cell) or exogenously, for example by administration to the culture medium.

In embodiments, the gene of interest encodes the product, or encodes an enzyme required in order to produce said product.

The gene of interest may be homologous (endogenous) or heterologous. Preferably the gene of interest is heterologous.

In embodiments, the gene (or the gene of interest) is operatively linked to a promoter. Such promoters can control the expression of the product or enzyme encoded by the gene of interest (GOI), e.g. the homologous (endogenous) or heterologous product or enzyme. Thus, in some embodiments such promoters can be endogenous promoters (e.g. promoters that are native to the gene or cell) or can be constitutive promoters in order to enable continuous or constant expression. Alternatively, such promoters can be inducible or heterologous promoters, which can for example be used to enable the induction of the production of the product encoded by the gene in the cell at a desired time point.

Appropriate inducible promoters would be well known and described in the art, and any of these could be used. For example, in embodiments, the promoter can be pH-sensitive, light-sensitive, temperature-sensitive, or chemically-sensitive. Further embodiments and details of such promoters and systems are discussed elsewhere herein (including embodiments as described herein in relation to promoters for control of site-specific recombinases—such embodiments may be applied as embodiments of promoters for control of the gene of interest mutatis mutandis). An exemplary system is also shown in the Examples.

In other embodiments, expression of the gene (gene of interest) or the promoter may be induced or controlled by targeted proteolysis or by other appropriate techniques such as CRISPR interference (CRISPRi).

The concept of CRISPR interference (CRISPRi) is well known in the art and is described for example in Lei et al. 2013 (Cell 152, 1173-1183). In CRISPRi, a catalytically inactive version of Cas9 is targeted to a specific DNA sequence using sgRNA with a specific sequence. The catalytically inactive Cas9 binds to the target DNA region (for example a promoter region) to block RNA polymerase binding and transcript elongation.

Hence, in embodiments of the cell wherein expression of the gene (gene of interest) or the promoter is induced or controlled by CRISPR interference (CRISPRi), the cell comprises a gene encoding a catalytically inactive Cas9 and an sgRNA for targeting the catalytically inactive Cas9 to the gene or promoter for producing the desired product such that expression of the gene or promoter is inhibited.

In embodiments, the gene of interest and/or product is homologous.

The term “homologous” is generally understood in the art. Hence, a “homologous gene of interest” or a “homologous product” in the context of the invention is a gene of interest or a product that is normally (i.e. naturally or natively) present in or produced by the cell (or cell line or strain). Hence, for a cell to possess a homologous gene of interest or produce a homologous product, it may not need to be exposed to a heterologous substrate. Hence “homologous” can mean “endogenous”.

In embodiments, the gene of interest is a homologous gene of interest, wherein the gene of interest is located in a non-native position within the cell.

Where the product is a homologous or endogenous product, the cell may already produce or be capable of producing the product before the excising of the nucleic acid sequence(s) from the chromosome and the forming of the extra-chromosomal DNA molecule. In this instance, the excising of the nucleic acid sequence(s) from the chromosome and the forming of the extra-chromosomal DNA molecule can be triggered in order to increase the levels of product or to overexpress the product, e.g. to increase the titer, yield and/or productivity of the cell in producing the homologous or endogenous product, e.g. compared to a control cell, as discussed elsewhere herein.

Preferably, the product is not produced (or not produced in significant amounts, or not produced in measurable amounts) before the excising and forming steps have taken place. Preferably, alternatively or in addition, the gene of interest is not expressed (or not expressed in significant amounts, or not expressed in measurable amounts) before the excising and forming steps have taken place.

In alternative and more preferred embodiments, the gene of interest and/or the product is heterologous.

The term “heterologous” is generally understood in the art. Hence a “heterologous gene of interest” or a “heterologous product” in the context of the invention is a gene of interest or a product that is not normally (i.e. not naturally or not natively) present in or produced by the cell (or cell line or strain). Hence, for a cell to possess a heterologous gene of interest or produce a heterologous product it may need to be modified genetically or to be exposed to a heterologous substrate. Hence “heterologous” can mean “exogenous”. The term “heterologous” contrasts with “homologous” and “endogenous”, which are also generally understood in the art.

Preferably, the product is not produced (or not produced in significant amounts, or not produced in measurable amounts) before the excising and forming steps have taken place. Preferably, alternatively or in addition, the gene of interest is not expressed (or not expressed in significant amounts, or not expressed in measurable amounts) before the excising and forming steps have taken place.

In embodiments, the nucleic acid sequence(s) (to be excised from the chromosome) comprise more than one (or multiple, e.g. two, three, four or five) genes of interest, and/or the extra-chromosomal DNA molecule comprises more than one (or multiple, e.g. two, three, four or five) genes of interest. The genes of interest may be the same or different.

A cell of the invention (e.g. a modified cell of the invention) can be generated using standard techniques in the art, for example using recombinases, for example using homologous recombination, for example using the phage lambda Red recombinase system (see for example Progress in Biophysics and Molecular Biology 147 (2019) 33e46). Such homologous recombination using recombinases can for example be carried out using a vector comprising the genetic elements of interest, for example a vector comprising a gene of interest and an origin of replication (or two origins of replication) flanked by site-specific recombination sites, wherein the site-specific recombination sites are themselves flanked by (i.e. nested within) nucleotide sequences homologous to the corresponding sequence in a (or the) chromosome of the cell of interest. A cell of the invention (e.g. a modified cell of the invention) can also be generated using non-homologous site-specific recombination. For example one can use the phage lambda integrase to integrate the cargo sequences into a naturally occurring attB site in the E. coli chromosome.

Thus, in one aspect, the invention provides a method of producing a cell of the invention.

In embodiments, the method of producing a cell of the invention comprises inserting one or more polynucleotide vectors as described herein into a (or the) chromosome of the cell. Preferably said one or more polynucleotide vectors are inserted into a (or the) chromosome using recombinases, for example using homologous recombination, for example using the phage lambda Red recombinase system, or using non-homologous site-specific recombination, for example using phage lambda integrase.

In the process of generating a cell of the invention, a (or the) original (i.e. native, natural, wild-type or endogenous) origin of replication of the starting bacterial cell may be replaced with a different origin of replication. For example, the inserted origin of replication may have a different sequence (e.g. the origin of replication may be from a different cell line or strain, or may be an exogenous sequence) as compared to an (or the) original (i.e. native, natural, wild-type or endogenous) origin of replication of the starting bacterial cell. Alternatively and more preferably, a (or the) origin of replication is the same (i.e. the same native, natural, wild-type or endogenous) origin of replication, i.e. the polynucleotide fragment encoding the original origin of replication is replaced, via the homologous recombination or other replacement process, with another polynucleotide fragment encoding the same origin of replication.

Similarly, in the methods of producing product and other aspects of the invention described herein, a (or the) origin of replication may have a different sequence (e.g. the origin of replication may be from a different cell line or strain, or may be an exogenous sequence) as compared to a (or the) original (i.e. native, natural, wild-type or endogenous) origin of replication of the cell used.

As described further below, the method of the present invention may advantageously provide an increase or improvement in gene copy number, gene expression and/or product production. However, it should be noted that the method of the present invention may be useful even if the production level in the cells of the present invention is the same as in a cell of the art. This is because, as antibiotics are not needed in the method of the present invention, product purification and waste management, etc., become easier and therefore the methods become more efficient and cost effective.

In embodiments the method of the present invention increases or improves the level or amount or concentration of product or product production (or product produced) by the cell, preferably in terms of titer, yield or productivity, or in terms of titer per cell, yield per cell, or productivity per cell. Preferably said increases are measurable or significant increases, for example are statistically or clinically significant. By way of example, a cell of the invention which can give rise to increases of at least, or up to, 1.5 fold, 2 fold, 2.5 fold, 3 fold, 3.5 fold, 4 fold, 4.5 fold, 5 fold, 6 fold, 7 fold, 8 fold, 9 fold or 10 fold, in levels of product production or product produced (preferably in terms of titer, yield or productivity, or in terms of titer per cell, yield per cell, or productivity per cell) or in activity of the enzyme or enzymes which produces said product or has a positive role in producing said product, is preferred. Any appropriate comparison can be used, for example an increase when compared to said levels observed from the same type of cell when the excising of the nucleic acid sequence(s) from the chromosome and the forming of the extra-chromosomal DNA molecule is not induced (e.g. does not or cannot take place), or in comparison to said levels from a control cell or cell line or strain, e.g. as described elsewhere.

The term “control strain” (or control cell) as used herein in general can refer to a cell which does not have an extra-chromosomal DNA molecule as described herein, or does not have the capability to produce an extra-chromosomal DNA molecule as described herein. For example, “control strain” can refer to a cell which differs from the cell of the invention in that the control strain is unmodified, or the chromosome of the control strain is unmodified, or the control strain contains a native or wild-type or unmodified chromosome, e.g. is a native or wild-type or unmodified strain or cell.

Alternatively, the term “control cell” or control cell line” or “control strain” may indicate a cell (for example the cell of the invention) when used in conditions where the excising of the nucleic acid sequence(s) from the chromosome and the forming of the extra-chromosomal DNA molecule is not triggered/induced (for example a cell of the invention as defined herein).

Alternatively, the term “control cell” or control cell line” or “control strain” may indicate a cell expressing a product in accordance with a method of the art. For example, the control cell may be transformed with a conventional plasmid for production of the product, wherein the conventional plasmid requires antibiotic selection in order to be retained within the cell. For example, the conventional plasmid may be an mRFP1-KanR plasmid as used in the Examples.

Appropriate methods of measuring levels of product production or product produced (preferably in terms of titer, yield or productivity, or in terms of titer per cell, yield per cell, or productivity per cell) or activity of the enzyme(s) which produces said product would be well known to a person skilled in the art. Thus, in some embodiments of the invention, the method will involve the step of detecting or determining the amount or level (e.g. the concentration) of product produced by a cell or cell line (e.g. when determining titer or yield), and optionally measuring the time taken to produce said amount or level (e.g. the concentration) of product produced by the cell.

Thus, typically the cells (or cell lines or strains) of the invention or for use in the methods of the invention will exhibit higher levels, sometimes significantly higher levels, of product production or product produced (preferably in terms of titer, yield or productivity, or in terms of titer per cell, yield per cell, or productivity per cell) after formation of the extra-chromosomal DNA molecule than a control cell as described above. If appropriate, the levels of product production can conveniently be measured or determined by methods known in the art. Thus, cells (or cell lines or strains) capable of higher (increased) levels, or significantly higher (increased) levels, of product production or product produced than a control (preferably in terms of titer, yield or productivity, or in terms of titer per cell, yield per cell, or productivity per cell), for example when assessed in vitro, form a yet further aspect of the invention.

The product produced by the cells may be secreted into the culture medium (or supernatant), or may be retained intracellularly. Therefore, the levels of production or product produced may be measured in terms of the level of product present in the culture medium (or supernatant), the level of product retained intracellularly (for example, this is measurable after standard protein extraction and purification), or both, as appropriate depending on the product concerned.

Viewed alternatively, cells (or cell lines or strains) capable of higher (increased) levels, or significantly higher (increased) levels, of product production or product produced when formation of the extra-chromosomal DNA molecule is triggered than a control cell (preferably in terms of titer, yield or productivity, or in terms of titer per cell, yield per cell, or productivity per cell), for example in the culture medium (or supernatant) of cells and/or intracellularly, for example when assessed in vitro, form a yet further aspect of the invention. For example, cells (or cell lines or strains) capable of higher (increased) levels, or significantly higher (increased) levels of product production or product produced in a subject when triggered (preferably in terms of titer, yield or productivity, or in terms of titer per cell, yield per cell, or productivity per cell), for example higher (increased) local levels of product production or product produced in a subject when excising and forming is triggered than a control cell, for example when assessed in vivo, form a yet further aspect of the invention, in particular where such an effect is observed when the cells are administered to a subject.

The cell of the present invention may be administered to the subject in any appropriate manner conventional for therapy, e.g. therapy using cells, and for the treatment of the indicated diseases, including but not limited to oral, sublingual, transdermal, cutaneous, rectal, nasal, vaginal, or ocular administration, or administration via inhalation or via buccal administration or by injection. Additionally, the cells or compositions of the present invention may be formulated for parenteral administration, for example by injection or continuous infusion. The route of administration may be any route that effectively transports the cell to the desired site without harming the recipient.

Preferably said higher levels or increases (in product production, or of product produced, or activity of the enzyme(s) which produces said product) in the cell are measurable or significant increases, for example are statistically or clinically significant. By way of example, cells (or cell lines or strains) which can give rise to increases of at least, or up to 10%, 20%, 30%, 40%, 50%, 60%, 70%, 80%, 90%, 100%, 150%, 200%, 250%, 300%, 350%, 400%, 450%, 500%, 550%, or higher, in levels of product or product production or product produced (preferably in terms of titer, yield or productivity, or in terms of titer per cell, yield per cell, or productivity per cell), for example local or in vitro levels of product production or product produced, or in levels of activity of the enzyme(s) which produces said product (for example when assessed in vitro), compared to the levels with a control as described above are preferred. Viewed alternatively, cells which can give rise to increases of at least, or up to, 1.5 fold, 2 fold, 2.5 fold, 3 fold, 3.5 fold, 4 fold, 4.5 fold, 5 fold, 6 fold, 7 fold, 8 fold, 9 fold, or 10 fold, in levels of product or product production or product produced (preferably in terms of titer, yield or productivity, or in terms of titer per cell, yield per cell, or productivity per cell), for example local or in vitro levels of product or product production or product produced, or in levels activity of the enzyme(s) which produces said product, compared to the levels with a control cell as described above are preferred. In vitro levels can conveniently be measured as described elsewhere herein, for example in cell cultures. Preferably, such increases are increases in extracellular (e.g. secreted) and/or intracellular levels of product (preferably in terms of titer or yield) or in activity of the enzyme(s) which produces said product, for example as measured in vitro, for example in the culture medium (or supernatant) of cell cultures, or intracellularly.

Appropriate methods for measuring the level of product or product production or product produced (preferably in terms of titer, yield or productivity, or in terms of titer per cell, yield per cell, or productivity per cell) or the activity of the enzyme(s) which produces said product are known to the skilled person and any of these can be used.

In embodiments the method of the present invention increases the level of product or product production or product produced by the cell (preferably in terms of titer, yield or productivity, or in terms of titer per cell, yield per cell, or productivity per cell). Preferably said increases are measurable or significant increases, for example are statistically or clinically significant. By way of example, it is preferred that the excising of the nucleic acid sequence(s) from the chromosome and the forming of the extra-chromosomal DNA molecule when used in the methods of the present invention gives rise to fold increases or percentage increases as defined above, in levels of product production or product produced (preferably in terms of titer, yield or productivity, or in terms of titer per cell, yield per cell, or productivity per cell) or in activity of the enzyme(s) which produces said product. Any appropriate comparison can be used, for example an increase in levels (preferably in terms of titer, yield or productivity, or in terms of titer per cell, yield per cell, or productivity per cell) when compared to the levels observed in the same cell (or same type of cell) when formation of the extra-chromosomal DNA molecule is not triggered, or the levels (preferably in terms of titer, yield or productivity, or in terms of titer per cell, yield per cell, or productivity per cell) when compared to the levels with a control cell, e.g. a control cell as described elsewhere herein.

Optionally, the level of product or product production or product produced (preferably titer, yield or productivity) by the cell can be quantified per number of cells (or per cell population size or per cell population density). In other words, the level of product or product production or product produced by the cell (preferably titer, yield or productivity) can be quantified in a manner which takes into account the number of cells (or cell population size or cell population density) used to produce the product.

Appropriate methods of measuring the cell population size or the numbers of cells are well known in the art including by measurement of optical density of the cell population, for example at 450 nm (OD₄₅₀) or 600 nm (OD₆₀₀) or 650 nm (OD₆₅₀).

Where the origin of replication(s) in the chromosome is (are) inactivated in the method of the invention (for example because the origin of replication is excised and forms part of the extra-chromosomal DNA molecule of the invention), the cell may exhibit a peak cell density which is different (higher or lower) to that of a cell which has not had its origin of replication removed.

In embodiments, the peak cell density (or maximum cell density, or maximal cell density, or cell density plateau) of a culture of the cell of the invention, or used in the methods of the invention, is at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, about 100% (or approximately the same, or the same), over 100%, at least 100%, at least 105%, at least 110%, about 110%, at least 115%, at least 120%, at least 125%, at least 130%, at least 135%, at least 140%, at least 145%, at least 150%, of the peak cell density the cell culture or of a control cell culture e.g. wherein excising and forming are not done or cannot occur. Values “up to” these percentages are also provided.

Preferably, the peak cell density (or maximum cell density, or maximal cell density, or cell density plateau) of a culture of the cell of the invention, or used in the methods of the invention, is at least 70%, at least 75%, at least 80%, at least 85%, at least 90%, at least 95%, at least 100%, about 100% (or approximately the same, or the same), over 100%, at least 100%, at least 105%, at least 110%, about 110%, at least 115%, or at least 120% of the peak cell density of a control cell culture e.g. wherein excising and forming are not done or cannot occur. Values “up to” these percentages are also provided.

Preferably, the peak cell density (or maximum cell density, or maximal cell density, or cell density plateau) of a culture of the cell of the invention, or used in the methods of the invention, is at least 90%, at least 95%, at least 100%, about 100% (or approximately the same, or the same) over 100%, at least 100%, at least 105%, at least 110%, or about 110%, of the peak cell density of a control cell culture e.g. excising and forming are not done or cannot occur. Values “up to” these percentages are also provided.

An appropriate control cell culture is as described elsewhere herein, and can for example be a culture of the cell of the invention wherein the excising of the nucleic acid sequence(s) from the chromosome and the forming of the extra-chromosomal DNA molecule is not induced (e.g. does not or cannot take place).

In embodiments, the level of product produced or level of production by a cell or modified cell of the invention, or used in the methods of the invention, is higher where the peak cell density (or maximum cell density, or maximal cell density, or cell density plateau) of the cell culture or modified cell culture is closer to that of a control cell culture, for example at least 70% of the peak cell density etc., of a control cell culture as described above.

In embodiments, the method of the invention increases the yield or titer of the product produced by the cell, or increases the productivity of the cell.

The term “titer” means the amount or concentration of product produced during or at the end of the bioprocess. Hence, titer can be measured in units of “g/L” or in any other appropriate units.

The term “yield” means the mass of product produced per mass of substrate (e.g. carbon and energy source) provided to the cells for the production method or bioprocess. Hence, yield can be measured in units of “g/g” or in any other appropriate units.

The term “productivity” relates to how quickly the cells are producing the product in the bioprocess. Hence, productivity is the amount or concentration of product produced per unit time. Hence, productivity can be measured in units of “g L⁻¹ h⁻¹”, i.e. grams per litre per hour or in any other appropriate units.

The cell may already produce the product before the formation of the extra-chromosomal DNA molecule is triggered. In this instance, triggering of the formation of the extra-chromosomal DNA molecule can be used to increase the levels of product or to overexpress the product, e.g. to increase the titer, yield and/or productivity of the cell in producing the homologous or endogenous product, e.g. compared to a control cell, as discussed elsewhere herein.

Preferably, the product is not produced (or not produced in significant amounts, or not produced in measurable amounts) before the excising and forming steps have taken place. Preferably, alternatively or in addition, the gene of interest is not expressed (or not expressed in significant amounts, or not expressed in measurable amounts) before the excising and forming steps have taken place.

In a typical method of the present invention, during the growth stage, the gene of interest is present in the genome in a single copy, and when the biomass has accumulated to the desired level, POPing is induced, resulting in the formation of an extra-chromosomal DNA molecule comprising an origin of replication and a gene of interest. This extra-chromosomal DNA molecule begins to replicate, and its copy number increases. Then, when induced, the gene of interest (and/or the desired product) is expressed at a level similar to or even exceeding the expression level from conventional expression plasmids. The product may then (or thereby) be produced, and then the product may be collected (or isolated).

The copy number of a sequence or genetic element may be expressed in the form of a ratio with respect to the copy number of the genome. This copy number ratio may be calculated for example by the technique described in the Examples or another appropriate method known in the art.

Thus, the extra-chromosomal DNA molecule copy number (or the extra-chromosomal DNA molecule to genome copy number ratio) may reach at least 2, 3, 4, 5, 6, 7, 8, 9 or 10 during the method of the invention, preferably at least 5. Alternatively, the copy number (or copy number ratio) may reach a level at least the same as a control cell, for example a control cell comprising a conventional expression plasmid using antibiotic selection as described herein. In embodiments, expression of the gene of interest may be induced once this copy number (or copy number ratio) is reached.

The activity of the agents responsible for triggering the excising and forming steps (also sometimes referred to herein as “POPing”) and optionally the inactivation of the origin of replication (“switching”) may be controlled to avoid premature formation of the extra-chromosomal DNA molecule and subsequent loss during the growth of the cells. For example, the inactivation may be induced or controlled through control of an agent responsible for or involved in (e.g. catalyzing) the excising and/or forming steps. For example, the excising and/or forming steps may be induced or controlled via a promoter which is operatively linked to a gene encoding an agent responsible for or involved in the excising and forming (“POPing”) steps. The excising and/or forming steps can then be triggered by induction of said promoter. Thus, preferred promoters for this are inducible promoters.

In embodiments of the invention comprising the excision of multiple nucleic acid sequence(s) from a (or the) chromosome, the nucleic acid sequences may be excised (or the excision events may be triggered) at different times. In embodiments where the method of the invention comprises a step of inactivating all of the origins of replication in the cell, this inactivation event (“switching”), and the excision of the nucleic acid sequence(s) which will go on to form part(s) of the extra-chromosomal DNA molecule (“POPing”), may be triggered at different times. However, preferably the switching and POPing take place at the same time. This may be achieved for example by placing the gene(s) responsible for switching and POPing under the control of the same promoter. More preferably, the switching and POPing may take place as part of the same excision event.

In embodiments, the method of the invention comprises: inducing expression of the gene of interest and/or inducing replication of the extra-chromosomal DNA molecule. Inducing replication of the extra-chromosomal DNA molecule may alternatively be referred to as activating an (or the) origin of replication of (or when it is part of) the extra-chromosomal DNA molecule; further embodiments in this regard are discussed elsewhere herein. The induction of expression of the gene of interest and the induction of replication of the extra-chromosomal DNA molecule may take place simultaneously or sequentially. If sequentially, they can be carried out in either order. The excising and forming steps may take place simultaneously or sequentially with one or both of the inducing steps.

Thus, in embodiments, the invention provides a method of producing a product by a cell, the method comprising:
excising one or more nucleic acid sequences from a chromosome in the cell, wherein the one or more nucleic acid sequences comprises a gene of interest and an origin of replication; and
forming an extra-chromosomal DNA molecule, wherein the extra-chromosomal DNA molecule comprises the excised gene of interest and an excised origin of replication;
inducing replication of the extra-chromosomal DNA molecule; and
inducing expression of the gene of interest from the extra-chromosomal DNA molecule.

Optionally, the method (and other methods of the invention described herein) may further comprise a step of (or may conclude with a step of, or have a final step of) collecting the product, for example as described elsewhere herein.

Thus, in embodiments, the invention provides a method of producing a product by a cell, the method comprising:
excising one or more nucleic acid sequences from a chromosome in the cell, wherein the one or more nucleic acid sequences comprises a gene of interest and an origin of replication; and
forming an extra-chromosomal DNA molecule, wherein the extra-chromosomal DNA molecule comprises the excised gene of interest and an excised origin of replication;
inducing expression of the gene of interest from the extra-chromosomal DNA molecule; and
inducing replication of the extra-chromosomal DNA molecule.

Optionally, the method (and other methods of the invention described herein) may further comprise a step of (or may conclude with a step of, or have a final step of) collecting the product, for example as described elsewhere herein.

In embodiments (for example of the one or more nucleic acid sequence(s), and/or of the extra chromosomal DNA molecule, of the method of the invention), the gene of interest and a gene for (or responsible for) controlling the replication of an (or the) origin of replication (e.g. the trfA gene) are operatively linked to (or under the control of) the same promoter.

Preferably, the expression of the gene of interest, and/or the replication of the extra-chromosomal DNA molecule, is induced (or activated) by formation of the extra-chromosomal DNA molecule. This may be achieved by designing the nucleic acid sequence(s) to be excised from the chromosome which will form the extra-chromosomal DNA molecule, such that, when the extra-chromosomal DNA molecule is formed (or through its formation), a promoter is operatively linked with the gene of interest and/or a gene for (or responsible for) controlling activity of an (or the) origin of replication.

This operative linkage of promoter to gene is typically achieved by joining said promoter sequence to said gene sequence, for example by ligating one or more of the excised nucleic acid sequences together to form the extra-chromosomal DNA molecule. Preferably the operative linkage may be achieved by circularization of an excised nucleic acid sequence (or where multiple excised nucleic acid sequences are joined and circularized) to form the extra-chromosomal DNA molecule described herein. More preferably, the operative linkage may be achieved where a promoter is recombined to the front (or upstream) of the gene of interest when the extra-chromosomal DNA molecule is formed. A list of possible strong promoters which may be used for this purpose include: tac promoter, trc promoter, hybrid P3 promoter, Pveg promoter, PlacIq1 promoter and J23101 promoter. However, any strong non-inducible (or constitutive) promoter may be used.

In embodiments, the expression of the gene of interest is higher (or is increased, or increases, or increases at a timepoint) after the excising and forming steps of the method of the invention have been performed. In embodiments, the gene of interest is not expressed (and/or the product is not produced) before the excising and forming steps of the method of the invention have been performed, but the gene of interest is expressed (and or the product is produced) after (or expression of the gene of interest is increased after, or expression of the gene of interest increases after, or expression of the gene of interest increases at a timepoint after) the excising and forming steps of the method have been performed. In embodiments, the gene of interest is not expressed (and/or the product is not produced) before the excising and forming steps of the method of the invention have been performed, and/or the gene of interest is expressed (and or the product is produced) after (or expression of the gene of interest is increased after, or expression of the gene of interest increases after, or expression of the gene of interest increases at a timepoint after) the excising and forming steps of the method have been performed.

It is advantageous to not express the gene of interest while it is in the chromosome because constant chromosomal recombinant protein expression has several drawbacks. For example, constant chromosomal recombinant protein expression drains resources and so cells may grow slower; and also generates selection pressure against high expression, so it is possible for non-expressing mutants to take over.

When it is stated that the gene of interest is “not expressed”, this term is also contemplated to include scenarios where the gene of interest is not significantly expressed, or not measurably expressed.

Preferably, the threshold optical density level after, e.g. immediately after, which excising of the nucleic acid sequence(s) from the chromosome and forming of the extra-chromosomal DNA molecule should be initiated or induced is at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of the peak cell density (or maximum cell density, or maximal cell density, or cell density plateau) of the cell culture after the excising and forming steps, or of a control cell culture, e.g. of the peak cell density of the cell culture in the absence of said excising and forming steps.

Preferably, the threshold optical density level after, e.g. immediately after, which excising of the nucleic acid sequence(s) from the chromosome and forming of the extra-chromosomal DNA molecule should be initiated or induced is between 1 to 5%, 5 to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, 70 to 75%, 75 to 80%, 80 to 85%, or 85 to 90% of the peak cell density (or maximum cell density, or maximal cell density, or cell density plateau) of the cell culture after said excising and forming steps, or of a control cell culture, e.g. of the peak cell density of the cell culture in the absence of said excising and forming steps. For example, the threshold optical density level after, e.g. immediately after, which excising of the nucleic acid sequence(s) from the chromosome and forming of the extra-chromosomal DNA molecule should be initiated or induced is 2-5%.

The optical density level for the peak or maximum etc., cell density as discussed above and elsewhere herein can be predetermined but will vary for different cells under different conditions, for example different growth conditions such as media and temperature, or the production of different products. Thus, using a percentage of maximum cell density that is achieved under chosen or desired conditions is particularly convenient to determine when excising of the nucleic acid sequence(s) from the chromosome and forming of the extra-chromosomal DNA molecule should be initiated or induced.

The control cell culture can for example be a culture of the cell of the invention wherein excising of the nucleic acid sequence(s) from the chromosome and forming of the extra-chromosomal DNA molecule is not induced (e.g. does not or cannot take place).

In embodiments, the cell exists within a cell population. Indeed, typically the cells are part of, or comprise or consist of, a cell population.

In preferred methods of the invention, the excising of the nucleic acid sequence(s) from the chromosome and the forming of the extra-chromosomal DNA molecule of the method of the invention take place during the exponential growth phase of the cell population. The appropriate time point during the exponential growth phase to carry out (or trigger) the excision and forming steps can readily be determined, e.g. for desired or maximum product production. For example, in some embodiments, it may be beneficial to carry out (or trigger) the excision and forming steps at the beginning (or in the first half) of the exponential growth phase where the cell density is low (or relatively low), or, in some embodiments, at a later part (e.g. in the second half, e.g. at or towards the end of) of the exponential growth phase where the cell density is high (e.g. higher or significantly higher than at the beginning of the exponential growth phase). The optimal timing for the excising of the nucleic acid sequence(s) from the chromosome and the forming of the extra-chromosomal DNA molecule may depend on the final (or maximum) cell density it is desired to achieve. If the desired final cell density is high then it may be desirable to induce the excising and forming later in the exponential growth phase, whereas if the desired final cell density is low (or does not need to be so high) then the excising and forming may be induced early in the exponential growth phase.

It is well understood in the art that the growth of cells in culture can typically be modelled with four different phases: (1) lag phase, (2) exponential phase, (3) stationary phase, and (4) death phase. During the lag phase, cells adapt to culture conditions, and minimal or no cell division occurs. During the exponential phase, cell division occurs and so the number of cells doubles with each consecutive time period. The stationary phase is reached due to a growth-limiting factor, for example the depletion of an essential nutrient. During the stationary phase the rate of cell growth matches the rate of cell death (for example, both can be effectively non-existent), i.e. the number of cells remains approximately the same. During or at the death phase, more cells die, and so the number of (living) cells decreases.

It is understood in the art that the time at which the exponential growth phase begins can either be predicted/known in advance (e.g. through knowledge of the growth pattern of the cell or cell line or strain in question under specific conditions/inoculation levels) or by regularly monitoring growth, for example by measurement of optical density of the cell culture, for example at 450 nm (OD₄₅₀) or 600 nm (OD₆₀₀) or 650 nm (OD₆₅₀).

In the methods of the present invention, the process of a cell changing from growth stage to production stage may be induced by (or may be caused by, or may be concomitant with) the the steps of excising of the nucleic acid sequence(s) from the chromosome and forming of the extra-chromosomal DNA molecule as described herein.

Expression of the gene of interest (and/or production of the product, i.e. the desired product) or induction of expression of the gene of interest (and/or induction of production of the desired product) may take place at any appropriate time point, for example at the same time as, or after, or by, formation of the extra-chromosomal DNA molecule.

In embodiments, expression of the gene of interest (and/or production of the desired product) may be initiated or induced at the same time that the excising of the nucleic acid sequence(s) from the chromosome and the forming of the extra-chromosomal DNA molecule is induced or carried out (or at approximately the same time as the excising and forming, or immediately before excising and forming, or immediately after excising and forming, or shortly before or after excising and forming).

The production of the desired product may be initiated for example by inducing production and/or activation of the enzyme(s) or metabolic pathway(s) responsible for generation of the desired product or otherwise inducing production of the product, e.g. where the product is a protein. Preferably said desired product, and/or said enzyme(s), is encoded by the gene of interest as described herein. Production is therefore initiated for example by inducing expression of the nucleotide sequence encoding the product (and/or by inducing expression of the gene of interest).

The method of the invention may be viewed as comprising a step of producing the product.

In embodiments where the gene of interest encodes the product, the step of producing the product may be achieved by (or may be the same as) expressing the gene of interest from the extra-chromosomal DNA molecule (or may be achieved by (or may be the same as) the step of inducing the expression of the gene of interest from the extra-chromosomal DNA molecule). In embodiments where the gene of interest does not encode the product, for example where the gene of interest encodes an enzyme for producing the product, the step of producing the product may be achieved after (or shortly after, or directly after, or as a consequence of) expressing the gene of interest from the extra-chromosomal DNA molecule (or may be achieved after (or shortly after, or directly after, or as a consequence of) the step of inducing the expression of the gene of interest from the extra-chromosomal DNA molecule). Thus, the expression of the gene of interest may lead directly or indirectly to producing the product.

In embodiments, the invention provides a method of producing a product by a cell, the method comprising:
excising one or more nucleic acid sequences from a chromosome in the cell, wherein the one or more nucleic acid sequences comprises a gene of interest and an origin of replication; and
forming an extra-chromosomal DNA molecule, wherein the extra-chromosomal DNA molecule comprises the excised gene of interest and an excised origin of replication;
inducing expression of the gene of interest from the extra-chromosomal DNA molecule, thereby producing the product (e.g. directly producing the product or indirectly producing the product); and optionally
collecting the product.

In embodiments, the invention provides a method of producing a product by a cell, the method comprising:
excising one or more nucleic acid sequences from a chromosome in the cell, wherein the one or more nucleic acid sequences comprises a gene of interest and an origin of replication; and
forming an extra-chromosomal DNA molecule, wherein the extra-chromosomal DNA molecule comprises the excised gene of interest and an excised origin of replication;
inducing expression of the gene of interest from the extra-chromosomal DNA molecule;
producing the product (e.g. directly producing the product or indirectly producing the product); and optionally
collecting the product.

Alternatively, the timing of initiation or induction of the gene of interest (and/or the timing of initiation or induction of production of the desired product) may not necessarily be tied to the excising of the nucleic acid sequence(s) from the chromosome and the forming of the extra-chromosomal DNA molecule, and can be carried out at any appropriate time point providing the cells are capable of measurable or significant expression of the gene of interest (or of producing measurable or significant levels of the product). For example, in embodiments, production of product is initiated or induced (for example, by inducing production and/or activation of the enzyme(s) or metabolic pathway(s) responsible for generation of the desired product or otherwise inducing production of the product, e.g. where the product is a protein, by inducing expression of the gene or nucleic acid sequence encoding the product) during (or approximately at the beginning, or immediately before the beginning, or immediately after the beginning, or shortly before the beginning, or shortly after the beginning of) the exponential growth phase of the cell population. In other words, product production may for example be induced or carried out in the first half of the exponential growth phase, or at a time point at or before a cell density of 50% of the maximal cell density (or OD) is achieved. In other embodiments, product production may for example be induced or carried out later during the exponential growth phase of the cell population where the cell density is higher, for example in the second half of the exponential growth phase, or at a time point after a cell density of 50% of the maximal cell density (or OD) is achieved. Preferably said desired product, and/or said enzyme(s), is encoded by the gene of interest as described herein.

Alternatively, induction of expression of the gene of interest (and/or production of desired product) may not necessarily be tied to the timing of the exponential phase. For example, the gene of interest may be constitutively or endogenously expressed (and/or the product may be constitutively or endogenously produced), in which case no step of initiation or induction is necessarily required; or initiation or induction may be started before or after the beginning of the exponential phase of cell culture growth.

In alternative embodiments, expression of the gene of interest (and/or production of the desired product) is initiated or induced at other time points during the exponential growth phase; or even during another phase, for example during the lag phase (for example, it is possible to induce expression (and/or production) upon or after, e.g. immediately after, dilution of the cells into fresh medium from overnight culture), or during the stationary phase.

Hence, expression of the gene of interest (and/or product production) may be initiated or induced either at a predicted or convenient or suitable time after inoculation of cells into the culture medium, or after, e.g. immediately or soon after, the optical density of the cell culture reaches a certain or desired threshold level.

Preferably, the threshold optical density level at which, or after which (or immediately after which or soon after which), expression of the gene of interest should be initiated or induced (and/or product production should be initiated or induced) is at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of the peak cell density (or maximum cell density, or maximal cell density, or cell density plateau) of the cell culture, or of the cell culture after the excising of the nucleic acid sequence(s) from the chromosome and the forming of the extra-chromosomal DNA molecule, or of a control cell culture, e.g. of the peak cell density of the cell culture in the absence of said excising and forming.

Preferably, the threshold optical density level at which, or after which (or immediately after which or soon after which), expression of the gene of interest should be initiated or induced (and/or product production should be initiated or induced) is between 1 to 5%, 5 to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, 70 to 75%, 75 to 80%, 80 to 85%, or 85 to 90% of the peak cell density (or maximum cell density, or maximal cell density, or cell density plateau) of the cell culture, or of the cell culture after the excising of the nucleic acid sequence(s) from the chromosome and the forming of the extra-chromosomal DNA molecule, or of a control cell culture, e.g. of the peak cell density of the cell culture in the absence of excising and forming.

The optical density level for the peak or maximum etc., cell density as discussed above and elsewhere herein can be predetermined but will vary for different cells under different conditions, for example different growth conditions such as media and temperature, or the production of different products. Thus, using a percentage of maximum cell density that is achieved under chosen or desired conditions is particularly convenient to determine when product production (and/or the expression of the gene of interest) should be initiated or induced.

The control cell culture can for example be a culture of the cell of the invention wherein the excising of the nucleic acid sequence(s) from the chromosome and the forming of the extra-chromosomal DNA molecule is not induced (e.g. does not or cannot take place).

It will be appreciated that in some embodiments it is not necessary for the user to induce expression of the gene of interest (and/or production of the product) and that this step can be optional, for example because expression of the gene of interest (and/or induction or production of the product) takes place automatically in culture at a certain time point. This may be the case for example where production of the product is linked to a promoter which is sensitive to internal stimuli or internal conditions. For example, the promoter may be induced when the cell culture enters a specific phase of growth, for example as in the case of PCP_2836 promoter (Ma et al., 2018, Microbial Cell Factories. BioMed Central Ltd., 17 (1), p. 185. doi: 10.1186/s12934-018-1031-7.), phoPR promoter (Paul et al., 2004, Journal of Bacteriology. American Society for Microbiology Journals, 186 (13), pp. 4262-4275. doi: 10.1128/JB. 186.13.4262-4275.2004) and P170 promoter (Madsen et al., 1999, Molecular Microbiology. Blackwell Publishing Ltd., 32 (1), pp. 75-87. doi: 10.1046/j.1365-2958.1999.01326.x). Preferably, the promoter may be induced when the cell culture enters the exponential growth phase, or during the exponential growth phase. Other preferred settings include the use of auto-induction media (for example as described in Studier et al. 2005, Protein Expression and Purification, 2005, 41:207-234) and self-inducing systems (for example SILEX, as described in Briand et al. 2016, Scientific Reports, 6:33037).

In embodiments, the expression of the gene of interest is induced by formation of the extra-chromosomal DNA molecule. More preferably, as a result of formation of the extra-chromosomal DNA molecule, the gene of interest becomes operatively linked with a promoter, preferably a constitutive promoter, for example as in the POP8 design. This is advantageous, because this means that the human operator of the method does not need to actively induce the expression of the gene of interest (for example by inoculation of a substrate). It is also advantageous because the gene of interest is not expressed while in the chromosome, this is beneficial because constant chromosomal recombinant protein expression has several drawbacks. For example constant chromosomal recombinant protein expression drains resources and so cells may grow slower; and also generates selection pressure against high expression, so it is possible for non-expressing mutants to take over.

Alternatively, it may not be necessary for the user to induce production of the product (and/or expression of the gene of interest) in the cell if the product is produced constitutively or continuously or constantly, e.g. under the control of a constitutive or endogenous promoter, for example an endogenous product (although equally constitutive or continuous or constant production can be applied to the production of a heterologous product). In this case, the excising of the nucleic acid sequence(s) from the chromosome and the forming of the extra-chromosomal DNA molecule (and optionally also—whether as part of the excising and forming steps or separately—the irreversible inactivation of the origin of replication of the chromosome or all of the origins of replication of the chromosome) in the cell can allow an increase or enhancement in the level of expression of the gene of interest (and/or the increase in the production or levels of desired product), e.g. to increase the titer, yield and/or productivity of the cell in producing said product.

The methods may also further comprise the steps of collecting, isolating, purifying, harvesting or otherwise obtaining, the product from the cells, e.g. from the culture medium (or supernatant) or from the intracellular environment of the cell. Methods for carrying out such steps would be well known in the art.

In embodiments, the invention provides a method of producing a product by a cell, the method comprising:
excising one or more nucleic acid sequences from a chromosome in the cell, wherein the one or more nucleic acid sequences comprises a gene of interest and an origin of replication; and
forming an extra-chromosomal DNA molecule, wherein the extra-chromosomal DNA molecule comprises the excised gene of interest and an excised origin of replication;
inducing expression of the gene of interest from the extra-chromosomal DNA molecule; and
collecting the product.

Isolated or purified cells (or populations of cells) are also provided. In some embodiments such cells will not be, or will not correspond to, naturally occurring cell lines. Some embodiments will involve the further steps of culturing or propagating or producing such cell lines or strains and optionally formulating said cultured or propagated or produced cell lines or strains into a composition comprising said cell line or strain, e.g. a stable formulation for product production, e.g. for industrial or commercial product production, or into a pharmaceutical composition. Alternatively, such cells could be stored for future uses, for example through lyophilisation or freezing.

In another aspect, the invention provides a cell which is suitable for use in any of the aspects or embodiments of the method of the invention.

In another aspect, the invention provides a cell, wherein the cell comprises a chromosome comprising a gene of interest and an origin of replication, wherein the cell (or the chromosome) has been modified (e.g. genetically modified), such that the gene of interest and origin of replication can be excised from the chromosome and form an extra-chromosomal DNA molecule comprising the excised gene of interest and origin of replication (or such that the gene of interest and origin of replication can be excised from the chromosome and an extra-chromosomal DNA molecule comprising the excised gene of interest and the excised origin of replication can be formed).

In another aspect, the invention provides a cell, wherein the cell (or a chromosome within the cell) has been modified such that the cell comprises a chromosome comprising a gene of interest and an origin of replication, wherein the gene of interest and the origin of replication can be excised from the chromosome and an extra-chromosomal DNA molecule comprising the excised gene of interest and origin of replication can be formed.

In another aspect, the invention provides a cell, wherein the cell comprises a chromosome comprising a gene of interest and an origin of replication.

In another aspect, the invention provides a cell, wherein the cell comprises a chromosome comprising a nucleic acid sequence flanked by site-specific recombination sites for recognition by site-specific recombinase,
wherein said nucleic acid sequence comprises a gene of interest, an origin of replication, and a promoter, and wherein:
the promoter is positioned such that the promoter may be operatively linked with the gene of interest by excision and circularisation of the nucleic acid sequence by site-specific recombination using the site-specific recombination sites; and/or
the promoter is positioned such that the promoter may be operatively linked with a gene for regulating the activity of the origin of replication by excision and circularisation of the nucleic acid sequence by site-specific recombination using the site-specific recombination sites.

In embodiments, the chromosome comprises a nucleic acid sequence flanked by site-specific recombination sites for recognition by site-specific recombinase, wherein said flanked nucleic acid sequence comprises a gene of interest and an origin of replication. The flanked nucleic acid sequence may contain any of the components referred to in any of the aspects or embodiments described herein, for example any features referred to as being comprised within the one or more nucleic acid sequences to be excised from the chromosome as referred to elsewhere herein. Preferably the flanked nucleic acid sequence comprises a promoter positioned such that, if the flanked nucleic acid sequence were excised and circularised (e.g. to form an extra-chromosomal DNA molecule) by site-specific recombination using the site-specific recombination sites (i.e. the site-specific recombination sites flanking the nucleic acid sequence), then the promoter would thereby be operatively linked with a gene for regulating the activity of the origin of replication and/or be operatively linked with the gene of interest. Such a promoter is also described elsewhere herein.

In embodiments, the gene of interest and origin of replication are flanked by site-specific recombination sites for recognition by site-specific recombinase.

In embodiments, the site-specific recombination sites comprise site-specific recombination sites for recognition by a serine recombinase, preferably wherein the serine recombinase is γδ, Bxb1, φC31 or TP901, and/or wherein the site-specific recombination sites comprise site-specific recombination sites for recognition by a tyrosine recombinase, preferably wherein the tyrosine recombinase is Cre, Dre, Flp, KD, B2 or B3.

In embodiments, the site-specific recombination sites comprise a pair of site-specific recombination sites, and/or wherein the site-specific recombination sites comprise two or more pairs of site-specific recombination sites.

In embodiments, the cell further comprises a gene encoding the site-specific recombinase.

In embodiments, the gene encoding the site-specific recombinase (and/or or the gene of interest, and/or a gene for controlling the activity of the origin of replication) is operatively linked to a promoter. Preferably the promoter is temperature-sensitive, pH-sensitive, light-sensitive, or chemically-sensitive; more preferably the promoter is regulated by phage lambda cI857 repressor.

In another aspect, the invention provides a cell which is suitable for use in the method of the invention.

In another aspect, the invention provides a cell in which the excising and forming steps of the invention have been performed. Thus, the invention provides a cell comprising an extra-chromosomal DNA molecule as defined in any of the aspects or embodiments provided herein. Said cell may also comprise one or more chromosomes as defined in any of the aspects or embodiments provided herein after the excising and forming steps of the method of the invention have been performed; for example the cell may be a cell which does not comprise any functional origin of replication sequences in (any of) the chromosome(s) of the cell.

In another aspect, the invention provides a method of any of the aspects and embodiments herein, wherein said cell is as defined in any one of aspects and embodiments herein.

Cells of the invention as described herein can sometimes be referred to as modified cells.

In another aspect, the invention provides a polynucleotide vector comprising a gene of interest, an origin of replication, and site-specific recombination sites, wherein the site-specific recombination sites flank the gene of interest and the origin of replication. The polynucleotide vector may be inserted into a cell, for example by homologous recombination, in order to generate a cell of the invention which may be used in the method of the invention.

In another aspect, the invention provides a use of the cell or the polynucleotide vector of any of the aspects and embodiments provided herein, for producing a product. Exemplary products are described elsewhere herein.

The examples of the present specification use E. coli cells to demonstrate the invention. The functioning of extra-chromosomal DNA molecules such as plasmids is also characterised in other bacterial species, and also eukaryotic cells such as yeast, filamentous fungi and mammalian cells. Thus, in embodiments, the cell may be prokaryotic cell (for example a bacterial cell, preferably E. coli) or a eukaryotic cell (for example a yeast, fungal (e.g. filamentous fungal) or mammalian cell). In such embodiments, the components used are adapted or selected accordingly for function in the cell type chosen.

Certain cells (for example eukaryotic cells and certain bacterial cells) may possess more than one chromosome. Thus, when the invention is applied to such cells possessing multiple chromosomes, the method of the invention (including the excising and forming steps) typically applies to one of the chromosomes within the cell. However, in such cells, where an object of the method is to inactivate all the chromosomal origins of replication within the cell, there may be a need to inactivate (e.g. excise) origins of replication from more than one (typically all) chromosomes within the cell. Similarly, where a cell contains multiple chromosomal origins of replication (e.g. has multiple chromosomes, or a single chromosome with multiple origins of replication), then it may be needed to inactivate (e.g. excise) more than one (typically all) of the chromosomal origins of replication.

A cell which is capable of being used in the method of the invention (i.e. a cell wherein it is possible to carry out the excising and forming steps as described herein) may be a cell which has been modified in order to obtain this capability. For example, this capability may be conferred (i.e. a cell of the invention may be generated) by integrating a polynucleotide sequence (or multiple polynucleotide sequences) into the chromosome, for example wherein the polynucleotide sequence (or multiple polynucleotide sequences) comprises the gene of interest, an origin of replication and optionally a means for excising the polynucleotide sequences, e.g. flanking site-specific recombination sites. The polynucleotide sequence(s) may be integrated into the cell by a standard method in the art, for example recombineering or homologous recombination, for example using the phage lambda Red recombinase system (see for example Progress in Biophysics and Molecular Biology 147 (2019) 33e46) or the PCR and phage lambda Red recombinase system of Datsenko and Wanner (PNAS, 2000, 97 (12), 6640-6645). A cell of the invention can also be generated using non-homologous site-specific recombination as described elsewhere herein.

In embodiments, the cell of the invention can produce a product and/or express the gene of interest as described herein (or is capable of producing a product or is producing a product, and/or is capable of expressing the gene of interest or is expressing the gene of interest), e.g. a desired product or the gene of interest in accordance with the methods of the present invention. Examples of desired products and genes of interest are described elsewhere herein. Thus, a cell producing a product (preferably a product encoded by the gene of interest as described herein) is a yet further aspect of the invention.

In embodiments, the cell comprises a gene encoding the product or encoding an enzyme required in order to produce the product. Preferably the gene of interest encodes the product, or for example an enzyme for producing the product (e.g. an enzyme of a reaction pathway which produces (or leads to the production of) the product). Appropriate genes encoding products or enzymes are also described elsewhere herein, for example the product (and encoding gene) may be an endogenous, homologous, or heterologous product.

In embodiments, the gene (e.g. the gene encoding the product, or the gene of interest) is operatively linked to a promoter. Further embodiments of appropriate promoters are described elsewhere herein (including embodiments as described herein in relation to promoters for control of site-specific recombinases-such embodiments may be applied as embodiments of promoters for control of the gene of interest mutatis mutandis), and for example can be native or heterologous promoters, or can be constitutive or inducible promoters.

In another aspect, the invention provides a polynucleotide vector comprising an origin of replication and a gene of interest flanked by site-specific recombination sites, preferably wherein the gene of interest is a heterologous gene of interest and/or preferably wherein the gene of interest is not a selectable marker. Appropriate and preferred site-specific recombination sites are described elsewhere herein.

In embodiments of the method of the invention, the formation of the extra-chromosomal DNA molecule triggers the expression of (or an increase in the expression of) the gene of interest.

In the methods of (or uses in) producing product (product production methods) as described herein, any further steps required for product production can be included. For example, the cells producing the product may be cultured, grown or otherwise propagated or produced, by appropriate methods well known in the art in order for product, e.g. appropriate or sufficient amounts of product, to be produced, or for the population of cells to be grown or expanded. Thus, the methods may further comprise steps of culturing, growing or otherwise propagating the cells.

As the cells of the invention can be engineered to produce products of interest, including therapeutically effective and useful products, and in useful or significant quantities, such cells have clear therapeutic uses, for example can be administered to subjects or patients, in particular human patients, where administration of that product is therapeutically effective or useful. For example, use of cells as living medicines, e.g. probiotics, is accepted in the art and the use of the cells of the invention in any therapies where the use of such living medicines is suitable or appropriate are contemplated by the present invention.

Thus, in another aspect, the invention provides the cell of the invention, for use in therapy.

In another aspect, the invention provides the cell of the invention, for use in the treatment or prevention of a disease or pathology. In preferred embodiments, the disease or pathology is cancer, metabolic disease or an immunological disorder.

In another aspect, the invention provides the use of a cell of the invention, in the manufacture of a medicament for the treatment or prevention of a disease or pathology. In preferred embodiments, the disease or pathology is cancer, metabolic disease or an immunological disorder.

In another aspect, the invention provides a method of treatment or prevention of a disease or pathology comprising administering an effective amount of the cell of the invention, to a subject, preferably wherein the disease or pathology is cancer, a metabolic disease, or an immunological disorder.

In embodiments, the disease or pathology is a metabolic disease (or metabolic disorder). Preferably, the metabolic disease (or metabolic disorder) is an acid-base imbalance, a metabolic brain disease, a calcium metabolism disorder, a DNA repair-deficiency disorder, a glucose metabolism disorder, hyperlactatemia, an iron metabolism disorder, a lipid metabolism disorder, a malabsorption syndrome, metabolic syndrome, an inborn error of metabolism, a mitochondrial disease, a phosphorus metabolism disorder, a porphyria, a proteostasis deficiency, a metabolic skin disease, wasting syndrome (or cachexia), or a water-electrolyte imbalance.

In embodiments, the disease or pathology is an immunological disorder (or an immunological disease). Preferably, the immunological disorder (or immunological disease) is allergy, asthma, an autoimmune disease (for example lupus, scleroderma, hemolytic anemia, vasculitis, type 1 diabetes, Graves' disease, rheumatoid arthritis, multiple sclerosis, Goodpasture syndrome, Pernicious anemia, myopathy or Lyme disease), an autoinflammatory syndrome or an immunological deficiency syndrome.

The term “subject” (or “patient”) as used herein includes any mammal, for example humans and any livestock, domestic or laboratory animal. Specific examples include mice, rats, pigs, cats, dogs, horses, sheep, rabbits, cows and monkey (or other primate). Preferably, however, the patient is a human subject.

In embodiments relating to therapeutic methods and uses described herein, appropriate subjects are those having, suspected of having, or at risk of having (or susceptible to) the disease or pathology to be treated.

The administration of the cells or cell lines in said therapeutic methods and uses of the invention is carried out in pharmaceutically, therapeutically, or physiologically effective amounts, to subjects in need of treatment. Thus, said methods and uses may involve the additional step of identifying a subject in need of treatment.

Treatment of disease or pathology in accordance with the present invention (for example treatment of pre-existing disease) includes cure of said disease or conditions, or any reduction or alleviation of disease (e.g. reduction in disease severity) or symptoms of disease.

The methods and uses of the prevent invention are suitable for prevention of diseases as well as active treatment of diseases (for example treatment of pre-existing disease). Thus, prophylactic treatment is also encompassed by the invention. For this reason in the methods and uses of the present invention, treatment also includes prophylaxis or prevention where appropriate.

In embodiments, the cell is a bacterial cell, preferably a Gram-negative bacterial cell, or alternatively a Gram-positive bacterial cell.

In embodiments, the bacterial cell is Escherichia sp., Bacillus sp., Lactococcus sp., Streptococcus sp., Lactobacillus sp., Corynebacterium sp., Streptomyces sp., Pseudomonas sp., Clostridium sp., Xanthomonas sp, or Enterobacteriaceae.

In embodiments, the bacterial cell is Escherichia sp., more preferably Escherichia coli.

The term “plasmid” refers to an extra-chromosomal DNA molecule often in the form of circular double-stranded DNA. Such elements can be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence of interest or relevance, e.g. for a selected gene product, along with appropriate 3′ untranslated sequence into a cell.

As used throughout the application, the terms “a” and “an” are used in the sense that they mean “at least one”, “at least a first”, “one or more” or “a plurality” of the referenced components or steps, except in instances wherein an upper limit is thereafter specifically stated.

In addition, where the terms “comprise”, “comprises”, “has” or “having”, or other equivalent terms are used herein, then in some more specific embodiments these terms include the term “consists of” or “consists essentially of”, or other equivalent terms. Methods comprising certain steps also include, where appropriate, methods consisting of these steps.

The term “increase” or “enhance” (or equivalent terms) as described herein includes any measurable increase or elevation when compared with an appropriate control. Appropriate controls would readily be identified by a person skilled in the art and appropriate examples are described herein. Preferably the increase will be significant, for example clinically or statistically significant, for example with a probability value of ≤0.05, when compared to an appropriate control level or value.

The term “decrease” or “reduce” (or equivalent terms) as described herein includes any measurable decrease or reduction when compared with an appropriate control. Appropriate controls would readily be identified by a person skilled in the art and appropriate examples are described herein. Preferably the decrease will be significant, for example clinically or statistically significant, for example with a probability value of ≤0.05, when compared to an appropriate control level or value.

Methods of determining the statistical significance of differences between test groups of subjects or differences in levels or values of a particular parameter are well known and documented in the art. For example herein a decrease or increase is generally regarded as statistically significant if a statistical comparison using a significance test such as a Student t-test, Mann-Whitney U Rank-Sum test, chi-square test or Fisher's exact test, one-way ANOVA or two-way ANOVA tests as appropriate, shows a probability value of ≤0.05.

The invention will now be further described in the following non-limiting Examples with reference to the following figures.

### FIGURE LEGENDS

FIG. 1. The design of POP2. The GOI (mRFP1) is inserted into bacterial chromosome at a specific location that has been pre-modified. In the chromosome the GOI is in close vicinity to plasmid origin of replication, oriV and trfA gene, that is necessary for plasmid replication. However, trfA is lacking a promoter, is not transcribed and hence, oriV is not active. When integrase expression is induced by temperature shift from 30° C. to 37° C., the pre-determined fragment between attP and attB sites is excised from the genome and circularized, forming a POP2 plasmid. During plasmid formation a functional promoter is placed in front of the trfA gene, the TrfA protein is expressed and initiates plasmid replication from oriV. This leads to the elevated plasmid copy-number and increased production of POI.

FIG. 2. Excision of POP2 plasmid results in increased plasmid copy-number and protein expression. A. qPCR analysis of genomic and POP2 plasmid specific amplicons to determine copy-number ratios. The plasmid to genome copy numbers were compared in genomic mRFP1 strain in which the plasmid-specific amplicon remains in the genome, in POP2 strain in which POP2 plasmid is excised from the chromosome upon temperature shift, and in wild type E. coli strain carrying a self-replicating mRFP1-KanR plasmid. The plasmid and genome-specific amplicon ratios before temperature shift and 6 hours after temperature shift are presented. The average of three biological replicas with standard deviation is plotted. B. The assessment of protein production capability of POP2 strain compared with chromosomal and conventional plasmid-based expression. Cultures were seeded from overnight cultures at 1000× dilution and pre-grown at 30° C. (white plot area) on a microplate incubator. At timepoint zero hours the temperature was changed to 37° C. (grey plot area) to induce POP2 plasmid excision in the POP2 strain. The production of fluorescent mRFP1 protein was induced by adding HSL at 2 hours after temperature shift and monitored by the increase in fluorescence. The average of three biological replicas with standard deviation is plotted. C. The growth curves of the mRFP1 protein expression experiment. D. SDS-PAGE analysis of protein expression in wild type E. coli, genomic mRFP1 strain, POP2 and wild type E. coli carrying mRFP1-KanR. The samples were collected 14 hours after induction of mRFP1 synthesis and visualized on 12% SDS acrylamide gel. Purified 6His-mRFP1 was used as a control to confirm the identity of mRFP1 on SDS-PAGE.

FIG. 3. The design of POP1, POP3A, POP3B, and POP4. In all setups oriC is removed from the chromosome during plasmid pop-out. A. POP1 plasmid contains both oriC and trfA. B. In POP3A plasmid contains the trfA gene, but oriC forms a separate minicircle. C. In POP3B, trfA gene remains in the chromosome and will be transcribed after POP3B plasmid excision; oriC forms a separate minicircle. D. POP4 plasmid contains oriC as the only origin of replication.

FIG. 4. POP1 leads to the highest protein expression level. A. qPCR analysis of plasmid to genome copy-number changes in genomic mRFP1, POP1, POP3A, POP3B, POP4 strains before (0 h) and after (6 h) plasmid excision. The average of three biological replicas with standard deviation is plotted. B. The assessment of protein production capability of wild type E. coli carrying mRFP1-KanR plasmid, POP1, POP3A, POP3B, and POP4 strains. The cultures were seeded from overnight cultures at 1000× dilution and pre-grown at 30° C. (white plot area) on a microplate incubator. At timepoint zero hours the temperature was changed to 37° C. (grey plot area) to induce plasmid excision in the POP strains. The production of fluorescent mRFP1 protein was induced by adding HSL at 2 hours after temperature shift and monitored by the increase in fluorescence. The average of three biological replicas with standard deviation is plotted. C. The growth curves of the mRFP1 protein expression experiment. D. SDS-PAGE analysis of protein expression in wild type E. coli and POP1, POP3A and POP3B strains. The samples were collected 14 hours after induction of mRFP1 synthesis and visualized on 12% SDS acrylamide gel. Purified 6His-mRFP1 as control to confirm the identity of mRFP1 on SDS-PAGE.

FIG. 5. All POP designs are stable without antibiotics selection. The stability of POP strains was estimated by comparing the protein synthesis capability after 4 passages of 24 hours long preculturing to a 1 passage of 24 hours long preculturing. A. Growth curves and fluorescence from mRFP1 expression of cultures after 1 passage or 4 passages of 24 hours long preculturing. The cultures were seeded from the 1st or 4th passage at 1000× dilution and pre-grown at 30° C. (white plot area) on a microplate incubator. At timepoint zero hours the temperature was changed to 37° C. (grey plot area) to induce plasmid excision in the POP strains. The production of fluorescent mRFP1 protein was induced by adding HSL at 2 hours after temperature shift and monitored by the increase in fluorescence. The growth of culture was monitored by measuring the optical density at 650 nm. The average of two biological replicas with standard deviation is plotted. B. SDS-PAGE analysis of the mRFP1 protein expression in wild type E. coli and POP2, POP1, POP3A and POP3B strains. The samples were collected 14 hours after induction of mRFP1 synthesis. Two biological replicas of each strain and passage condition were visualized on 12% SDS acrylamide gels.

FIG. 6. scFv and hFGF-2 protein expression in POP1 setup. SDS-PAGE analysis of the scFv and hFGF-2 protein expression in wild type E. coli and POP1-scFv and POP1-hFGF-2 strains. The samples were collected 20 hours after induction of protein synthesis and visualized on 12% SDS acrylamide gel.

FIG. 7. The design of POP5, POP6 and POP7. pMB1-type replication origin without the RNAII promoter is placed adjacent to attP sites. When the fragment between attP and attB sites is excised from the genome and circularized, a functional promoter is placed in front of pMB1-type replication origin, the RNAII is synthesised and initiates plasmid replication from pMB1-type replication origin.

FIG. 8. POP6 leads to the highest plasmid to genome copy-number ratio. A. qPCR analysis of plasmid to genome copy-number changes in genomic mRFP1, POP5, POP6, POP7 strains before (0 h) and after (6 h) plasmid excision. The average of two biological replicas with standard deviation is plotted. B. The assessment of protein production capability of POP5, POP6 and POP7 strains compared with chromosomal expression. Cultures were seeded from overnight cultures at 500× dilution and pre-grown at 30° C. (white plot area) on a microplate incubator. At timepoint zero hours the temperature was changed to 37° C. (grey plot area) to induce plasmid excision in the POP strains. The production of fluorescent mRFP1 protein was induced by adding HSL at 2 hours after temperature shift and monitored by the increase in fluorescence. The average of three biological replicas with standard deviation is plotted. C. The growth curves of the mRFP1 protein expression experiment. D. SDS-PAGE analysis of protein expression in wild type E. coli, genomic mRFP1 strain and POP5, POP6, POP7 strains. The samples were collected 14 hours after induction of mRFP1 synthesis and visualized on 12% SDS acrylamide gel. Purified 6His-mRFP1 as control to confirm the identity of mRFP1 on SDS-PAGE.

FIG. 9. POP8 design enables automatic induction of GOI from the excised POP-plasmid. A. The design of POP8. The GOI (mRFP1) does not have a promoter when inserted to the genome. A promoter is located downstream and will be placed in front of the GOI after formation of POP plasmid. TrfA is expressed from the same mRNA as GOI. B. The assessment of protein production capability of genomic mRFP1 and POP8 strains. The cultures were seeded from overnight cultures at 1000× dilution and pre-grown at 30° C. (white plot area) on a microplate incubator. At timepoint zero hours the temperature was changed to 37° C. (grey plot area) to induce plasmid excision and mRFP1 expression in the POP8 strain. The production of fluorescent mRFP1 protein was monitored by the increase in fluorescence. The average of three biological replicas with standard deviation is plotted. C. The growth curves of the mRFP1 protein expression experiment. D. SDS-PAGE analysis of the mRFP1 protein expression in wild type E. coli and POP8 strain. The samples were collected 14 hours after induction of mRFP1 synthesis and visualized on 12% SDS acrylamide gel.

### EXAMPLES

### Materials and Methods

### Bacterial Strains, Plasmids, and Growth Medium

Strains and plasmids are listed in Table 1. E. coli DH5a and BW23473 were used for plasmid cloning and propagation. Genomic alterations and protein expression experiments were performed in E. coli MG1655. E. coli was grown in lysogeny broth (LB). If plasmid maintenance was required either during cloning or in case of plasmid control strain, the media was supplemented with the appropriate amount of antibiotics (100 μg/ml ampicillin, 25 μg/ml chloramphenicol, and 25 μg/ml kanamycin).

### DNA Manipulations

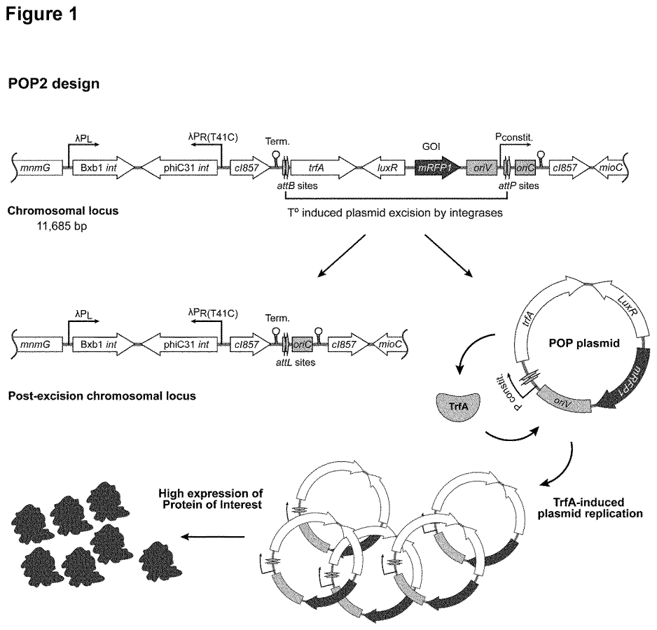

Short oligonucleotide sequences for cloning, sequencing, and qPCR analysis were ordered from Metabion International AG. Codon-optimised sequences of φC31 integrase with mutated λP_{R} (T41C) promoter, Bxb1 integrase with λPL promoter, cI857repressor with PkatG promoter, attB and attP sites with synthetic promoters and terminators were ordered as synthetic DNA fragments from Twist Bioscience (Table 2). trfA and oriV sequences were amplified from plasmid pAJ144. pMB1 family origins of replication were amplified without the RNAII promoter as follows: pBR322 ori sequences with and without bom and rop region were amplified from pAJ35; and pUC ori was amplified from pUC19. Codon-optimised sequences of scFv Herceptin (with C-terminal His-tag) and hFGF-2 (amino acids 135-288 with N-terminal 10His-tag and enterokinase cleavage site) were ordered as synthetic DNA fragments from Twist Bioscience. The fragments were assembled by overlap extension PCR, gel purified, and integrated into chromosome using recombineering (Datsenko and Wanner, PNAS, 2000, 97 (12), 6640-6645). The final modified genomic regions are illustrated in FIGS. 1, 3, 7 and 9, and their sequences are listed in Table 3. Plasmid mRFP1-KanR was constructed using CPEC method [1], the final sequence is listed in Table 4.

### Measurement of Copy Numbers

Cultures were grown overnight at 30° C. in a shaker-incubator, diluted 500× (POP1-POP4 and respective control strains) or 250× (POP5-POP7 and respective control strains) in a 15 mL of fresh medium, and grown for two more hours at 30° C. Then the temperature was shifted to 37° C. to induce POP formation and the switch (oriC excision) when applicable. The point of temperature shift is defined as timepoint 0 hours. Culture samples for copy number measurements were collected at timepoint 0 h and 6 h and stored at −20° C. freezer. OD650 was measured and equal amount of biomass (0.8 OD units) was subjected to quantitative PCR analysis. The 0.8 Units of cell pellets were resuspended in 150 μL of EDTA buffer (10 mM Tris, 0.1 mM EDTA pH 8), lysed for 10 min at 95° C., and serial dilution of 10×, 100×, 1000×, and 10000× of the cell lysate in PCR-grade water was analysed in qPCR assay using HOT FIREPol® Probe Universal qPCR Mix (Solis BioDyne). FAM-BHQ1 and HEX-BHQ1 labelled hydrolysis probes were designed to detect genomic- and plasmid-specific DNA sequences, respectively (Table 2). Reactions were mixed and run as by manufacturers recommendations using a LighCycler 480 System (Roche Diagnostics). The plasmid to genome copy number ratio was calculated using the following equation:

$$plasmid to genome ratio = 2 \hat{} ( {Ct}_{genome} - {Ct}_{plasmid} ) \qquad [ 2 ]$$

Plasmid to genome ratio was calculated for every dilution and averaged over dilutions.

### Measurement of Growth and Protein Production

Cultures of POP and control strains were grown overnight at 30° C. in a shaker-incubator, diluted 1000× (POP1-POP4 and POP8 and respective control strains) or 500× (POP5-POP7 and respective control strains) in fresh LB and continued to grow on a 96-well plate at 30° C. for two hours until the temperature was shifted to 37° C. (timepoint 0 h). The induction of mRFP1 synthesis was induced two hours after temperature shift by adding 1 μM N-(3-Oxooctanoyl)-L-homoserine lactone (HSL, Sigma-Aldrich, O1764, final concentration). The growth curves and mRFP1 related fluorescence, reflecting protein level, were determined using BioTek Synergy H1 plate reader. The optical density at 650 nm and fluorescence intensity (excitation at 580/13.5 nm and emission at 610/13.5 nm) were measured in a culture volume of 100 μL.

The amount of synthesised mRFP1 was estimated using SDS-PAGE. 14 hours after the induction of protein synthesis (16 hours after the temperature shift) 40 μL samples of POP and control cultures were collected and supplemented with 10 μL 5×SDS sample buffer. The samples were denatured at 98° C. for 10 minutes. 10 μL samples were resolved on 12% SDS-PAGE gel and visualised using InstantBlue® Coomassie Protein Stain (Abcam).

Cultures of POP1-scFv, POP1-hFGF-2 and control strains were grown overnight at 30° C. in a shaker-incubator, diluted 500× in fresh LB and continued to grow on a 96-well plate at 30° C. for two hours until the temperature was shifted to 37° C. After two hours of incubation at 37° C. the temperature was lowered to 26° C. and the synthesis of scFv and hFGF-2 proteins was induced by adding 1 μM HSL. 20 hours after the induction of protein synthesis, 40 μL sample of each culture was collected and supplemented with 10 μL 5×SDS sample buffer. The samples were denatured at 98° C. for 10 minutes. 10 μL samples were resolved on 12% SDS-PAGE gel and visualised using InstantBlue® Coomassie Protein Stain (Abcam).

TABLE 1
Strains and plasmids

| | Description | Soure/Reference |
|---|---|---|
| Strain | | |
| DH5α | F⁻ endA1 glnV44 thi-1 recA1 relA1 gyrA96 deoR | laboratory stock |
| | nupG purB20 φ80dlacZΔM15 Δ(lacZYA- | |
| | argF)U169, hsdR17(r_{K}⁻m_{K}⁺), λ⁻ | |
| MG1655 | K-12 F⁻ λ⁻ ilvG⁻ rfb-50 rph-1 | laboratory stock |
| POP1 strain | MG1655 ΔoriC:: λP_{L}-intBxB-λP_{R}(T41C)-intφC31- | This study |
| | PlacI-cl857-attP-trfA-luxR-mRFP1-oriV-oriC- | |
| | attB-PkatG-cl857(opt) | |
| POP2 strain | MG1655 ΔoriC:: λP_{L}-intBxB-λP_{R}(T41C)-intφC31- | This study |
| | PlacI-cl857-attB-trfA-luxR-mRFP1-oriV-attP- | |
| | oriC-PkatG-cl857(opt) | |
| POP3A strain | MG1655 ΔoriC:: λP_{L}-intBxB-λP_{R}(T41C)-intφC31- | This study |
| | PlacI-cl857-attB-trfA-luxR-mRFP1-oriV-attP-attP- | |
| | oriC-attB-PkatG-cl857(opt) | |
| POP3B strain | MG1655 ΔoriC:: λP_{L}-intBxB-λP_{R}(T41C)-intφC31- | This study |
| | PlacI-cl857-trfA-attB-luxR-mRFP1-oriV-attP-attP- | |
| | oriC-attB-PkatG-cl857(opt) | |
| POP4 strain | MG1655 ΔoriC:: λP_{L}-intBxB-λP_{R}(T41C)-intφC31- | This study |
| | PlacI-cl857-attP-luxR-mRFP1-oriC-attB-PkatG- | |
| | cl857(opt) | |
| genomic | MG1655::cat-luxR-mRFP1, Cm^{R} | This study |
| mRFP1 | | |
| POP5 strain | MG1655 ΔoriC:: λP_{L}-intBxB-λP_{R}(T41C)-intφC31- | This study |
| | PlacI-cl857-attP-pBR322ori-luxR-RFP- oriC-attB- | |
| | PkatG-cl857(opt) | |
| POP6 strain | MG1655 ΔoriC:: λP_{L}-intBxB-λP_{R}(T41C)-intφC31- | This study |
| | PlacI-cl857-attP-pBR322ori-bom-rop-luxR-RFP- | |
| | oriC-attB-PkatG-cl857(opt) | |
| POP7 strain | MG1655 ΔoriC:: λP_{L}-intBxB-λP_{R}(T41C)-intφC31- | This study |
| | PlacI-cl857-attP-pUCori-luxR-RFP- oriC-attB- | |
| | PkatG-cl857(opt) | |
| POP8 strain | MG1655 ΔoriC:: λP_{L}-intBxB-λP_{R}(T41C)-intφC31- | This study |
| | PlacI-cl857-attP-RFP- trfA-oriV-oriC-attB-PkatG- | |
| | cl857(opt) | |
| POP1-scFv | MG1655 ΔoriC:: λP_{L}-intBxB-λP_{R}(T41C)-intφC31- | This study |
| strain | PlacI-cl857-attP-trfA-luxR-scFv(opt)-oriV-oriC- | |
| | attB-PkatG-cl857(opt) | |
| POP1-hFGF-2 | MG1655 ΔoriC:: λP_{L}-intBxB-λP_{R}(T41C)-intφC31- | This study |
| strain | PlacI-cl857-attP-trfA-luxR-hFGF-2(opt)-oriV- | |
| | oriC-attB-PkatG-cl857(opt) | |
| Plasmid | | |
| pAJ144 | Tet^{R}, pJB_LuxR-6His-mRFP1 | [3] |
| pKD46 | Amp^{R}, λ-Red helper plasmid (recombinase) | [4] |
| mRFP1-KanR | Kan^{R}, oriV/RK2-trfA-luxR-mRFP1, | This study |
| pAJ35 | Kan^{R}, P_{lacI}_{^{−}}Cl857-λP_{R}(T41C)-intφC31 in pBR322 | Kasari et al., ACS |
| | | Synth. Biol. 2022, |
| | | 11, 2610-2622 |
| pUC19 | Amp^{R}, pUC_LacZα | New England |
| | | Biolabs |

^{a} Cm^{R}, chloramphenicol resistance; Kan^{R}, kanamycin resistance; Amp^{R}, ampicillin resistance; Tet^{R}, tetracycline resistance

TABLE 2
The nucleotide sequence of functional elements and sequences of oligonucleotides

| IntφC31attB(TT) | GTGCGGGTGCCAGGGCGTGCCCTTGGGCTCCCCGGGCGCGTACTCC | |
|---|---|---|
| | (SEQ ID NO: 1) | |
| IntφC31attP(TT) | AGTGCCCCAACTGGGGTAACCTTTGAGTTCTCTCAGTTGGGGGCGT | |
| | (SEQ ID NO: 2) | |
| IntφC31attB(AG) | GTGCGGGTGCCAGGGCGTGCCCAGGGGCTCCCCGGGCGCGTACTCC | |
| | (SEQ ID NO: 3) | |
| IntφC31attP(AG) | AGTGCCCCAACTGGGGTAACCTAGGAGTTCTCTCAGTTGGGGGCGT | |
| | (SEQ ID NO: 4) | |
| IntBxb attB(GA) | CGGCCGGCTTGTCGACGACGGCGGACTCCGTCGTCAGGATCATCCGGG | |
| | C (SEQ ID NO: 5) | |
| IntBxb attP(GA) | GTCGTGGTTTGTCTGGTCAACCACCGCGGACTCAGTGGTGTACGGTACA | |
| | AACCCA (SEQ ID NO: 6) | |
| IntBxb attB(GT) | CGGCCGGCTTGTCGACGACGGCGGTCTCCGTCGTCAGGATCATCCGGG | |
| | C (SEQ ID NO: 7) | |
| IntBxb attP(GT) | GTCGTGGTTTGTCTGGTCAACCACCGCGGTCTCAGTGGTGTACGGTACA | |
| | AACCCA (SEQ ID NO: 8) | |
| λP_{R}(T41C)ª | TATCACCGCAAGGGATAAATATCTAACACCGCGCGTGTTGACTATTTTAC | |
| | CTCTGGCGGTGATAATGGTTGCA (SEQ ID NO: 9) | |
| λP_{L} | AACCATCTGCGGTGATAAATTATCTCTGGCGGTGTTGACATAAATACCAC | |
| | TGGCGGTGATA (SEQ ID NO: 10) | |
| LuxR promoter | ACATAAGCACCTGTAGGATCGTACAGGTTTAGCGAAGAAAATGGTTTGTT | |
| from V. fischeri | ATAGTCGAATAA (SEQ ID NO: 11) | |
| PkatG | TAATCAAAAAAGCTTAATTAAGATCAATTTGATCTACATCTCTTTAACCAA | |
| | CAATATGTAAGATCTCAACTATCGCATCCGTGGATTAATTCAATTATAACT | |
| | TCTCTCTAACGCTGTGTATCGTAACGGTAACACTGTAGA (SEQ ID NO: 12) | |
| Pconstit. | TTGACAATTAATCATCGGCTCGTATAATG | promoter Ptac without lac |
| | (SEQ ID NO: 13) | operator |
| T0 (21imm) | TTGTTCAGAACGCTCGGTCTTGCACACC | transcriptinal terminator |
| | GGGCGTTTTTTCTTTGTGAGTCCA (SEQ | |
| | ID NO: 14) | |
| T (L3S3P25) | CCAATTATTGAAGCGGCTAACGCCGCTT | transcriptional terminator |
| | TTTTTGTTTCTGGTCTCCC (SEQ ID | |
| | NO: 15) | |
| T (L3S3P43) | TCTAACTAAAAACACCCTAACGGGTGTT | transcriptional terminator |
| | TTTTCTTTTCTGGTCTCCC (SEQ ID | |
| | NO: 16) | |
| probeAJ4 | 5′ HEX- | pairs with oAJ322, oAJ323, |
| | TCATCCGGGGTCAGCACCGTTTCTGCG | targets plasmid region |
| | GA-BHQ1 (SEQ ID NO: 17) | |
| probeAJ7 | 5′ 6-FAM- | pairs with oAJ327, oAJ328, |
| | CAGCCCTTTTGCACGATGGTGCGCA- | targets genomic region |
| | BHQ1 (SEQ ID NO: 18) | |
| oAJ322 | CGCTTGCGTTTTCCCTTGTC (SEQ ID | qPCR primer, targets plasmid |
| | NO: 19) | |
| oAJ323 | GCAGCCACGTAGAAAGCCA (SEQ ID | qPCR primer, targets plasmid |
| | NO: 20) | |
| oAJ327 | TGGTGCAACATTCACATCGT (SEQ ID | qPCR primer, targets genome |
| | NO: 21) | |
| oAJ328 | AGCGAAATCTGTGCCAACTT (SEQ ID | qPCR primer, targets genome |
| | NO: 22) | |

ªT41C mutation is indicated in bold

TABLE 3
Sequences of modified genomic regions of POP and control strains

POP1 strain (11269 bp) - SEQ ID NO: 23
POP2 strain (11685 bp) - SEQ ID NO: 24
POP3A strain (12328 bp) - SEQ ID NO: 25
POP3B strain (12331 bp) - SEQ ID NO: 26
POP4 strain (9493 bp) - SEQ ID NO: 27
genomic mRFP1 (4960 bp) - SEQ ID NO: 28
POP1-scFv strain (11263 bp) - SEQ ID NO: 29
POP1-hFGF-2 strain (11026 bp) - SEQ ID NO: 30
POP5 strain (10123 bp) - SEQ ID NO: 31
POP6 strain (11180 bp) - SEQ ID NO: 32
POP7 strain (10123 bp) - SEQ ID NO: 33
POP8 strain (10177 bp) - SEQ ID NO: 36

TABLE 4
Sequences of plasmids

mRFP1-KanR plasmid (5055 bp) - SEQ ID NO: 34

TABLE 5
Sequence of oriV used in the Examples

oriV (526 bp) - SEQ ID NO: 35

### Results

To replicate, plasmids need to contain a functional origin of replication (ori). Many different origins of replication with various modes of regulation have been described. These include, but are not limited to, pBR322, pMB1, ColE1, R6K, oriV, p15A, pSC101 and pUC [10]. Some require the presence of specific proteins for replication (in addition to cellular replication machinery), the former are encoded usually on the same plasmid. We took advantage of this fact and used oriV (originally from RK2 plasmid) in our design. oriV requires protein TrfA for replication and if TrfA is not expressed, oriV is inactive and does not initiate DNA replication.

We incorporate oriV, trfA gene, gene-of-interest and other desired components into E. coli chromosome (FIG. 1). These are flanked by serine recombinase recognition sites attB and attP. In this configuration trfA gene has no active promoter in front of it and it is not expressed. When we induce the expression of corresponding serine recombinase in the cells, it catalyses irreversible recombination between attB and attP sites, flipping out the DNA sequence in between by strand exchange, and re-joining the DNA into a hybrid attR/attL site [11]. As a result, a circular plasmid is formed, which is separated from the bacterial chromosome. This recombination event also brings a functional promoter to the front of trfA gene resulting in TrfA protein expression. TrfA initiates replication from oriV and the plasmid starts to replicate independently of chromosomal DNA replication. We call this setup a pop-out-plasmid (POP) and the design described on the FIG. 1 is POP2.

In these Examples, we are using serine recombinases from phages phiC31 and Bxb1 to build the POP, but other types of genome editing can also be used for the same purpose. Possible candidates include, but are not limited to, other serine recombinases [12], tyrosine recombinases [13], CRISPR/Cas [14] or any other type of enzyme that permanently removes the desired sequence from the chromosome and forms a circular plasmid.

The serine recombinase (or other type of editing) activity/expression may be controlled to avoid premature plasmid formation and subsequent loss during the growth of the cells. We used a temperature-sensitive phage lambda cI857 repressor for that purpose. At 30° C. (or lower) cI857 is bound to its target promoter (P_{R} or P_{L}) and represses it, while at 37° C. (or higher) the repression is relieved, and expression is turned on. Other types of gene expression control methods can possibly be used for the same purpose, including but not limited to, induction by inducers (IPTG, arabinose, homoserine lactones, anhydrotetracycline etc) [15], [16], [17], targeted proteolysis [18], different variants of CRISPR/Cas9 [19] etc.

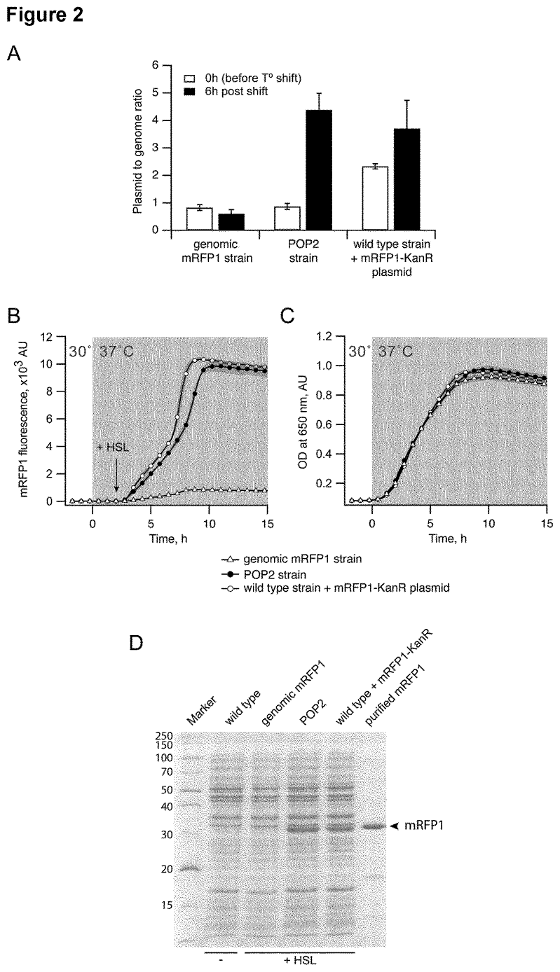

We tested POP formation by measuring the copy-number of a DNA sequence that is incorporated into POP plasmid and compared it to the copy-number of a DNA sequence that remains in the chromosome (FIG. 2A). When grown at 30° C. (until 0 h timepoint) both sequences are part of the E. coli chromosome, and their ratio remains close to 1. At six hours after temperature shift to 37° C. and POP2 formation the plasmid to genome ratio has increased fivefold, indicating a clear amplification of POP2 plasmid. At the same time in control strain, where both sequences stay on chromosome, the copy-number does not change. While five plasmid copies per copy of genome seems low, it is a natural copy-number of that particular plasmid configuration. As a proof we constructed a mRFP1-KanR plasmid that is similar to excised POP2 plasmid, transformed it into the wild type parental E. coli strain and determined that its copy number is similar to excised POP2 plasmid (FIG. 2A).

To assess how the formation of POP2 and the increase in copy-number affect the protein production capability we used a fluorescent reporter system that encodes for monomeric red fluorescent protein 1 (mRFP1) under the control of homoserine lactone (HSL) inducible promoter. By the design upon the POP2 excision event, the mRFP1 gene together with luxR transcriptional regulatory gene will become part of the POP2 plasmid (FIG. 1). Addition of HSL to the culture medium induces mRFP1 production which can be monitored by measuring red fluorescence (ex: 580 nm, em: 610 nm).

When a single-copy mRFP1 gene remains in the chromosome its expression level is very low (FIG. 2B). However, when mRFP1 gene is on the POP2, the fluorescence signal reaches ten times higher level indicating much higher protein expression at similar culture densities (FIG. 2B,C). In fact, it reaches the expression level of a conventional plasmid (mRFP1-KanR plasmid) that requires antibiotic selection. This is further corroborated by SDS-PAGE analysis where strong expression can be seen at lanes where mRFP1 is on the POP2 (FIG. 2D).

Our expression system does not need any selection for maintenance. In the POP design, during biomass growth the GOI remains integrated in the bacterial chromosome and is stably replicated in unison with the rest of the chromosome. When the POP plasmid is excised at the end of growth phase it will replicate to achieve a TrfA-defined copy-number. However, there are not enough cell divisions left for the POP plasmid to be lost by the imperfect segregation and lack of selection.

When cells keep dividing long enough after POP formation it is possible that the plasmid is lost from cells. To prevent that from happening we combined POP technology with our patent-pending Switcher Technology (Patent application WO2022117827A1) which enables to stop cell division and growth while keeping them in metabolically active state. Switcher Technology is based on controlled removal of oriC from the chromosome with the help of serine recombinases. Combining the two technologies led to two different designs: POP1 and POP3.

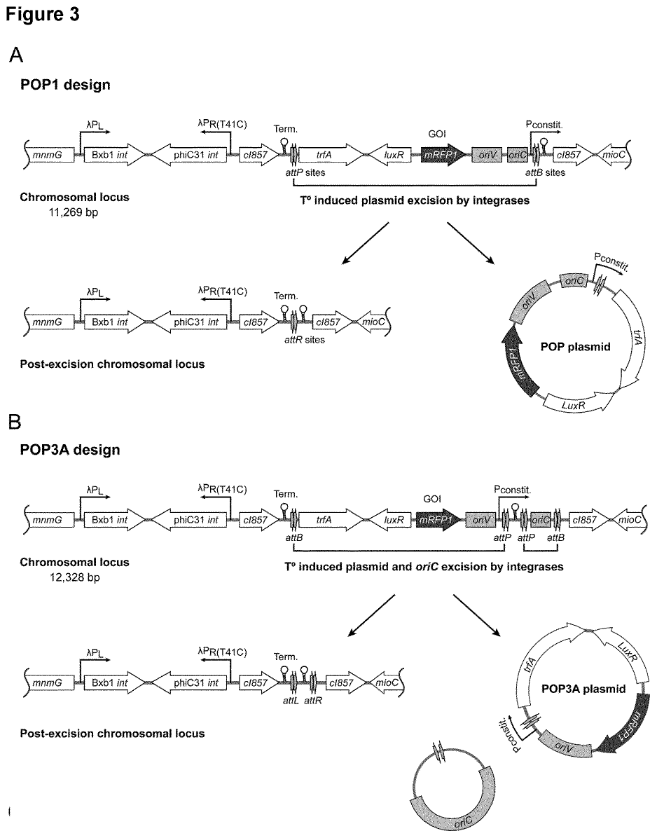

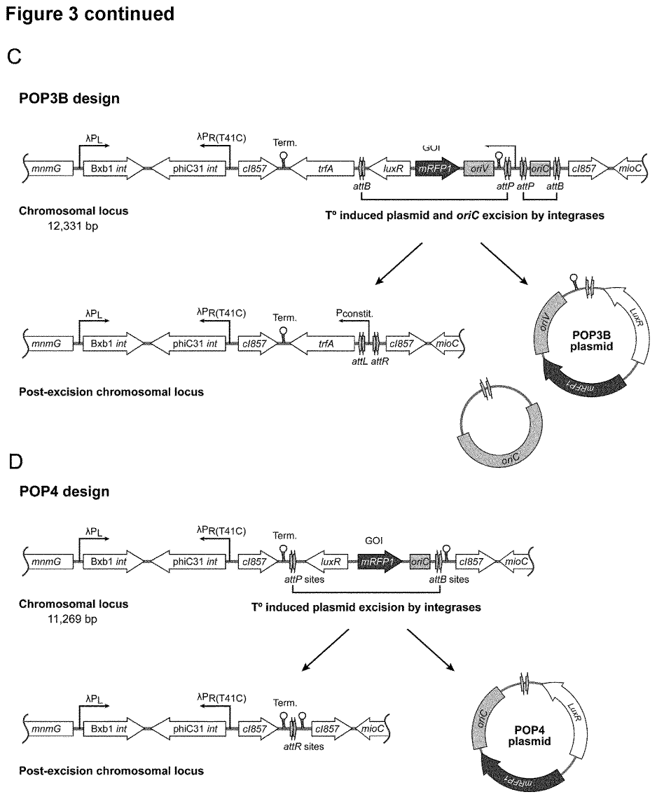

In the case of POP1 both POP and oriC are removed from the chromosome as a single unit (FIG. 3A). This formed plasmid contains both oriC and active TrfA-regulated oriV replication origin. As an alternative, in POP3 the excision of oriC and POP are two separate events, carried out by the same serine recombinases (FIG. 3B, C). POP3 plasmids contain only oriV without oriC; in POP3A the trfA gene is transferred to the POP plasmid and in POP3B it remains in the chromosome. Whichever location the trfA gene will be during POP induction, an active promoter will be recombined in front of it to induce the expression of TrfA protein. In POP4 no plasmid-specific origin of replication was introduced, only chromosomal oriC is part of the pop-out plasmid to drive its replication (FIG. 3D).

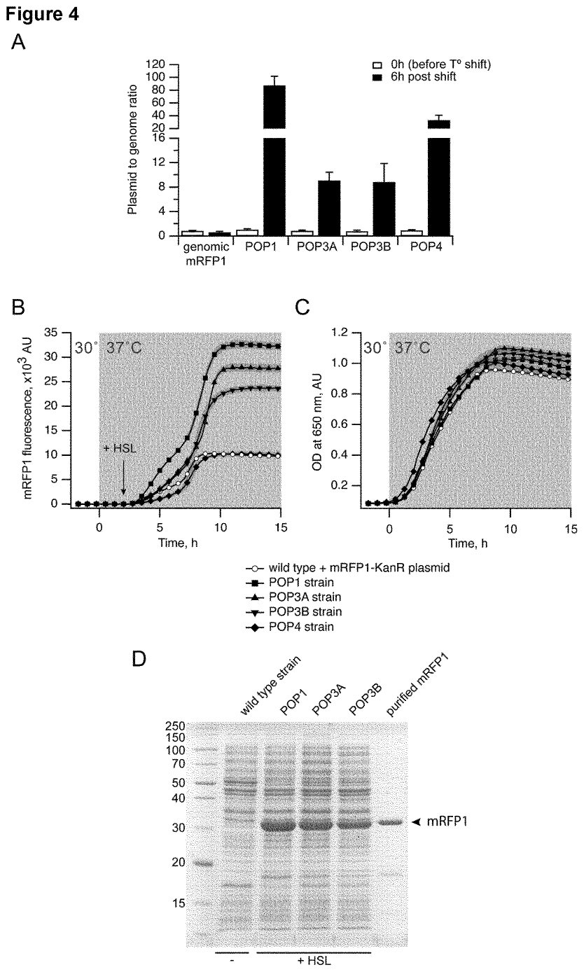

To assess the copy-number changes in POP1, POP3, and POP4 we carried out the qPCR-based measurements as before. 6 h after temperature shift there is a significant increase in copy-number to genome ratio of all POP versions (FIG. 4A). This proves the chromosome independent replication of the POP plasmids. The magnitude of the effect is at least partially due to the decrease in the relative abundance of the genome—after removal of oriC cells grow in length i.e. biomass increases, but the genome remains as a single copy per (elongated) cell.

Increased copy-number also leads to higher protein expression level. The highest mRFP1 expression level is measured in POP1 strain, followed by POP3A and POP3B (FIG. 4B) at similar culture densities (FIG. 4C). The expression in POP4 remained comparable to the control plasmid. The expression is again further corroborated by SDS-PAGE analysis (FIG. 4D).

The higher protein expression level in the case of POP1 and POP3 (compared to POP2) is probably achieved by two factors. First, higher copy-number increases gene dosage in the POP1 and POP3 strains. The second factor is that protein expression is elevated in cells in which oriC has been removed, even if other conditions are equal [20]. The high plasmid copy-number to genome ratio of POP4 does not lead to comparably high expression of mRFP1. The exact reason has remained elusive.

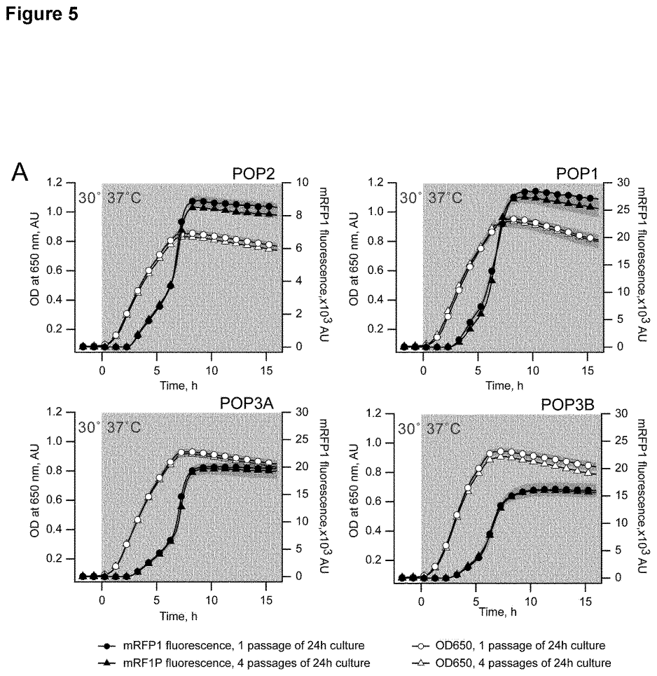

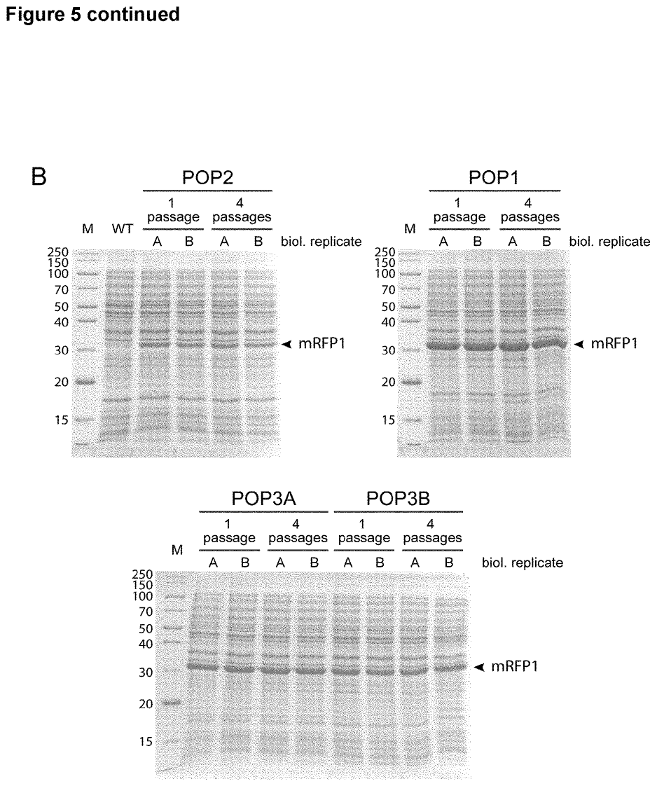

Long-term stability is very important property of any protein expression system used for industrial setup. Many cell doublings may occur before enough biomass is accumulated in a large-scale production. We mimicked the number of doublings needed for large-scale production by daily serial passages, making 1000 times dilution every day for four consecutive days. These passages were carried out without any selection. We tested the stability of our POP system by comparing protein expression in fourth day passage cultures to single-passage culture (FIG. 5). In both cultures the expression level of reporter protein mRFP1 was very similar, indicating a good stability of POP expression system (FIG. 5B). The same can be seen on SDS-PAGE analysis (FIG. 5C).

In conclusion, combining genomic stability of POP, the high copy-number of GOI upon request, and the physiology of Switcher cells result in antibiotic-free high-level protein expression system.

We have built the POP system in E. coli, but it should work in other bacteria as well and also in eukaryotes like yeasts, filamentous fungi and mammalian cells.

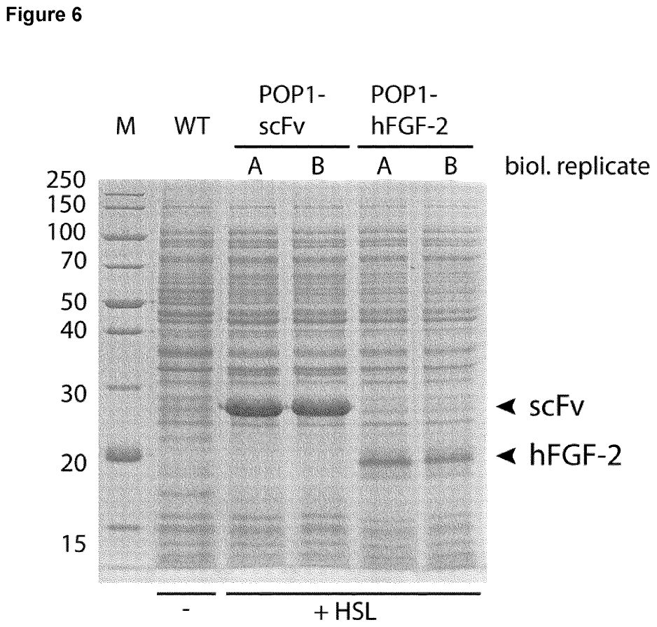

To confirm that the high protein production using the POP system is not purely mRFP1-specific, we tested the expression of two additional recombinant proteins in the POP1 design setup. The mRFP1 gene in POP1 strain was replaced by a gene encoding either single-chain fragment variable antibody (scFv Herceptin) or recombinant human fibroblast growth factor 2 (hFGF-2). Both proteins demonstrate a strong expression in the POP system (FIG. 6).

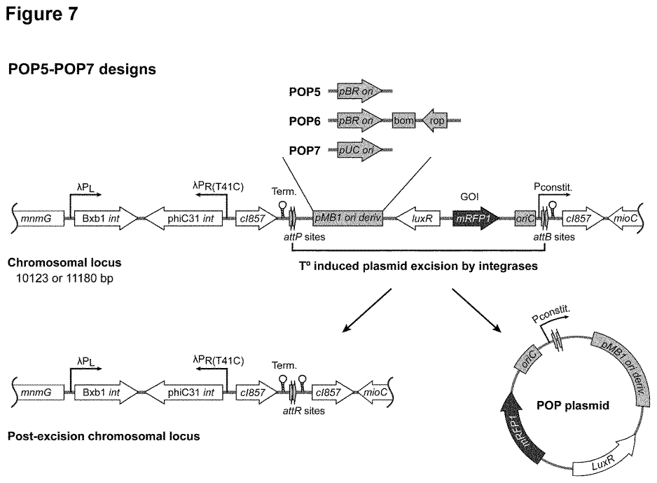

We wanted to confirm that the POP concept is not restricted to the plasmid types that's replication is Rep protein dependent like TrfA in RK2 plasmids. We replaced the trfA gene and RK2 oriV in the POP1 design with pMB1-type origins (G. Cesareni, D. W. Banner, Regulation of plasmid copy number by complementary RNAs, Trends in Biochemical Sciences, Volume 10, Issue 8, 1985, Pages 303-306). We incorporated pMB1 replication origin to the genome without a promoter in front of RNAII gene (FIG. 7). If RNAII is not synthesised the pMB1 replication origin is not active. Upon formation of POP plasmid, a strong constitutive promoter induces the transcription of RNAII and, thereby, enables plasmid replication. We constructed three POP strain variants to test the pMB1-type origins: POP5 design contains the pBR322 origin of replication; POP6 design contains the pBR322 origin of replication and the copy number controlling elements bom and rop; POP7 design contains the pUC origin of replication (FIG. 7).

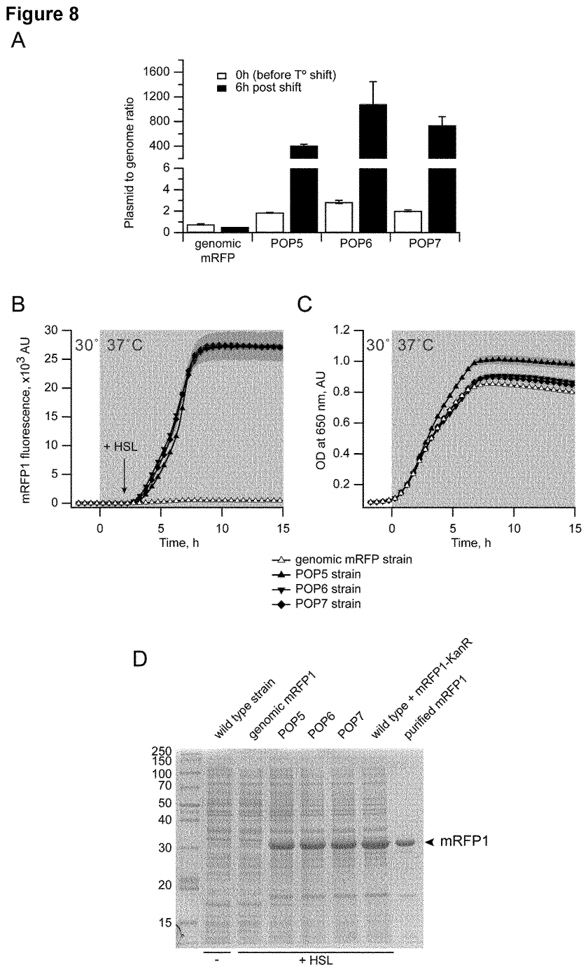

To determine the plasmid to genome DNA copy-number ratios of POP5, POP6 and POP7 strains, we carried out the qPCR-based measurements as before. We observed a significant increase in all plasmid-to-genome ratios in six hours after the temperature shift (FIG. 8A), the highest ratio being measured in POP6. As expected, the increased plasmid copy-numbers also lead to higher protein expression levels as compared to genomic expression. However, despite the 2-fold differences in the plasmid copy-numbers between the POP strains, the mRFP1 protein expression level remains comparably high (FIG. 8B) at similar culture densities (FIG. 8C). This was also confirmed by SDS-PAGE analysis (FIG. 8D). Other factors in cell physiology than the plasmid copy number will likely limit the protein production capability in this situation. In conclusion, the findings prove that the POP concept is functional for production of various proteins and can be designed using different plasmid replication mechanisms.

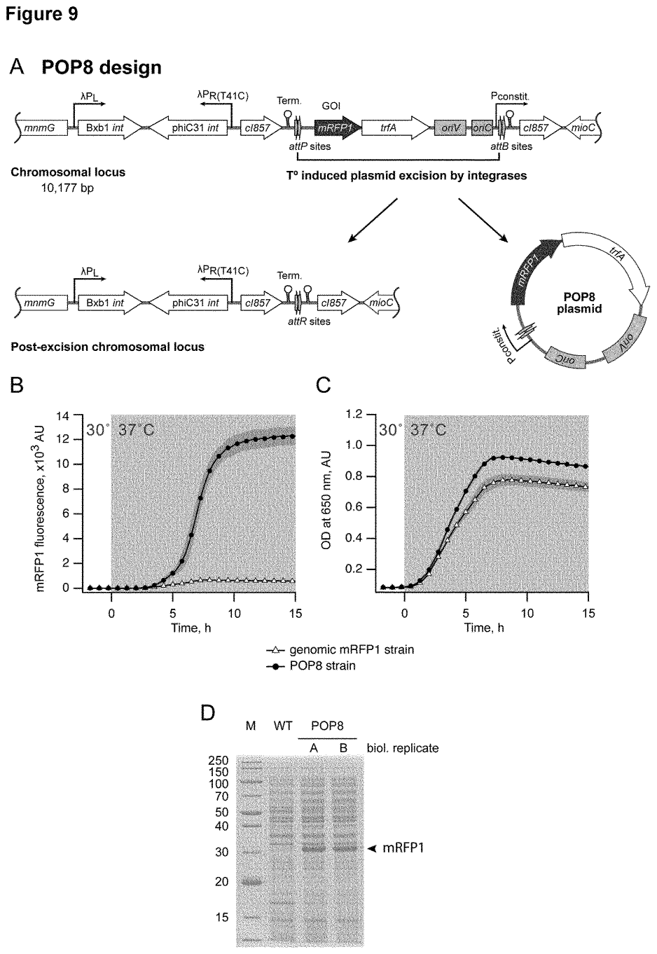

DNA re-arrangement during plasmid formation allowed us to design a new way to induce protein expression. In POP8 strain the mRFP1 gene does not have a promoter in front of it when it is in the genome (FIG. 9A). When the plasmid is excised from the genome and circularized, a constantly active promoter is located in the front of mRFP1 gene and the protein expression will start. Strong mRFP1 expression is detected by fluorescence measurements (FIG. 9B) and also on SDS-PAGE (FIG. 9D) after the temperature shift. This setup allows us to use any promoter as an inducible one and abandon small molecule inducers, like IPTG.

### REFERENCES

1. Gutterson N I, Koshland D E. Replacement and amplification of bacterial genes with sequences altered in vitro. Proceedings of the National Academy of Sciences of the United States of America. 1983; 80:4894. doi: 10.1073/PNAS.80.16.4894
2. Hersh M N, Ponder R G, Hastings P J, Rosenberg S M. Adaptive mutation and amplification in Escherichia coli: two pathways of genome adaptation under stress. Research in Microbiology. 2004; 155:352-359. doi: 10.1016/J.RESMIC.2004.01.020
3. Andersson D I, Hughes D. Gene Amplification and Adaptive Evolution in Bacteria. https://doi.org/101146/annurev-genet-102108-134805. 2009; 43:167-195. doi:10.1146/ANNUREV-GENET-102108-134805
4. Mignon C, Sodoyer R, Werle B. Antibiotic-Free Selection in Biotherapeutics: Now and Forever. Pathogens 2015, Vol 4, Pages 157-181. 2015; 4:157-181. doi:10.3390/PATHOGENS4020157
5. Oliveira P H, Mairhofer J. Marker-free plasmids for biotechnological applications-implications and perspectives. Trends in Biotechnology. 2013; 31:539-547. doi:10.1016/J.TIBTECH.2013.06.001
6. Wild J, Hradecna Z, Szybalski W, Blattner F R, Helinski D, Valla S, et al. Conditionally Amplifiable BACs: Switching From Single-Copy to High-Copy Vectors and Genomic Clones. Genome Research. 2002; 12:1434-1444. doi:10.1101/GR.130502
7. Wild J, Szybalski W. Copy-Control Tightly Regulated Expression Vectors Based on pBAC/oriV. Methods in molecular biology (Clifton, NJ). 2004; 267:155-167. doi:10.1385/1-59259-774-2:155
8. Yoon Y G, Cho J H, Kim S C. Cre/loxP-mediated excision and amplification of large segments of the Escherichia coli genome. Genetic Analysis: Biomolecular Engineering. 1998; 14:89-95. doi:10.1016/S1050-3862 (97) 10005-5
9. Pósfai G, Koob M, Hradečná Z, Hasan N, Filutowicz M, Szybalski W. In vivo excision and amplification of large segments of the Escherichia coli genome. Nucleic Acids Research. 1994; 22:2392-2398. doi:10.1093/NAR/22.12.2392
10. Solar G del, Giraldo R, Ruiz-Echevarría M J, Espinosa M, Díaz-Orejas R. Replication and Control of Circular Bacterial Plasmids. Microbiology and Molecular Biology Reviews. 1998; 62:434. doi:10.1128/MMBR.62.2.434-464.1998
11. Merrick C, Zhao J, Rosser S. Serine Integrases: Advancing Synthetic Biology. ACS Synthetic Biology. 2018. doi:10.1021/acssynbio.7b00308
12. Yang L, Nielsen A A K, Fernandez-Rodriguez J, McClune C J, Laub M T, Lu T K, et al. Permanent genetic memory with >1-byte capacity. Nature Methods. 2014; 11:1261-1266. doi:10.1038/nmeth.3147
13. Fogg P C M, Colloms S, Rosser S, Stark M, Smith M C M. New Applications for Phage Integrases. Journal of Molecular Biology. 2014; 426 VN-: 2703-2716. doi:10.1016/j.jmb.2014.05.014
14. Yao R, Liu D, Jia X, Zheng Y, Liu W, Xiao Y. CRISPR-Cas9/Cas12a biotechnology and application in bacteria. Synthetic and Systems Biotechnology. KeAi Communications Co.; 2018. pp. 135-149. doi:10.1016/j.synbio.2018.09.004
15. Lutz R, Bujard H. Independent and Tight Regulation of Transcriptional Units in Escherichia Coli Via the LacR/O, the TetR/O and AraC/11-12 Regulatory Elements|Nucleic Acids Research|Oxford Academic. Nucleic Acids Research. 1997; 25:1203-1210.
16. Marschall L, Sagmeister P, Herwig C. Tunable recombinant protein expression in E. coli: promoter systems and genetic constraints. Applied Microbiology and Biotechnology. 2017; 101:501-512. doi:10.1007/s00253-016-8045-z
17. Cox R S, Surette M G, Elowitz M B. Programming gene expression with combinatorial promoters. Mol Syst Biol. 2007; 3:145. doi:10.1038/msb4100187
18. Cameron D E, Collins J J. Tunable protein degradation in bacteria. Nat Biotechnol. 2014; 32:1276-1281. doi:10.1038/nbt.3053
19. Qi L S, Larson M H, Gilbert L A, Doudna J A, Weissman J S, Arkin A P, et al. Repurposing CRISPR as an RNA-γuided platform for sequence-specific control of gene expression. Cell. 2013; 152:1173-1183. doi:10.1016/j.cell.2013.02.022
20. Kasari M, Kasari V, Karmas M, Jõers A. Decoupling Growth and Production by Removing the Origin of Replication from a Bacterial Chromosome. ACS Synthetic Biology. 2022; 11:2610-2622. doi:10.1021/ACSSYNBIO.1C00618/SUPPL_FILE/SB1C00618_SI_010.TXT

1. A method of producing a product by a cell, the method comprising:
excising one or more nucleic acid sequences from a chromosome in the cell, wherein the one or more nucleic acid sequences comprises a gene of interest and an origin of replication;
forming an extra-chromosomal DNA molecule, wherein the extra-chromosomal DNA molecule comprises the excised gene of interest and an excised origin of replication; and
inducing expression of the gene of interest from the extra-chromosomal DNA molecule.

2. The method of claim 1, wherein the expression of the gene of interest from the extra-chromosomal DNA molecule is induced by formation of the extra-chromosomal DNA molecule.

3. The method of claim 1, wherein the expression of the gene of interest from the extra-chromosomal DNA molecule is induced at the same time as, or after, formation of the extra-chromosomal DNA molecule.

4. The method of any one of the preceding claims, wherein the gene of interest is a heterologous gene of interest.

5. The method of any one of the preceding claims, wherein the gene of interest encodes the product or an enzyme required for producing said product.

6. The method of any one of the preceding claims, wherein the one or more nucleic acid sequences does not comprise a selectable marker.

7. The method of any one of the preceding claims, wherein the one or more nucleic acid sequences comprises a second origin of replication.

8. The method of claim 7, wherein the extra-chromosomal DNA molecule comprises the excised second origin of replication.

9. The method of any one of the preceding claims, wherein the origin of replication or the second origin of replication is an endogenous or non-inducible origin of replication.

10. The method of any one of the preceding claims, wherein as a result of the method, the chromosome does not comprise a functional origin of replication.

11. The method of any one of the preceding claims, wherein the excising step and the forming step take place simultaneously or sequentially.

12. The method of any one of the preceding claims, wherein the extra-chromosomal DNA molecule is a plasmid.

13. The method of any one of the preceding claims, wherein the one or more nucleic acid sequences is one nucleic acid sequence.

14. The method of any one of the preceding claims, comprising inducing replication of the extra-chromosomal DNA molecule.

15. The method of claim 14, wherein the replication of the extra-chromosomal DNA molecule is induced by formation of the extra-chromosomal DNA molecule.

16. The method of claim 14 or claim 15, wherein the excising and forming steps take place simultaneously or sequentially with one or both of the inducing steps.

17. The method of any one of the preceding claims, wherein the cell exists within a cell population, and wherein the excising step and/or the forming step take place during the exponential growth phase of the cell population; preferably wherein the cell is a bacterial cell which exists within a bacterial cell population, and wherein the excising step and/or the forming step take place during the exponential growth phase of the bacterial cell population.

18. The method of any one of the preceding claims, wherein the origin of replication and/or the second origin of replication is non-inducible; preferably a chromosomal origin of replication or oriC; more preferably Escherichia coli oriC.

19. The method of any one of the preceding claims, wherein the origin of replication and/or the second origin of replication is inducible; preferably a plasmid origin of replication or oriV; more preferably the origin of replication or oriV of plasmid RK2, pBR322, pMB1, ColE1, R6K, p15A, pSC101 or pUC.

20. The method of any one of the preceding claims, comprising collecting the product.

21. A cell, wherein the cell comprises a chromosome comprising a nucleic acid sequence flanked by site-specific recombination sites for recognition by site-specific recombinase,
wherein said nucleic acid sequence comprises a gene of interest, an origin of replication, and a promoter, and wherein:
the promoter is positioned such that the promoter may be operatively linked with the gene of interest by excision and circularisation of the nucleic acid sequence by site-specific recombination using the site-specific recombination sites; and/or
the promoter is positioned such that the promoter may be operatively linked with a gene for regulating the activity of the origin of replication by excision and circularisation of the nucleic acid sequence by site-specific recombination using the site-specific recombination sites.

22. The cell of claim 21, wherein the site-specific recombination sites comprise site-specific recombination sites for recognition by a serine recombinase, preferably wherein the serine recombinase is γδ, Bxb1, φC31 or TP901, and/or wherein the site-specific recombination sites comprise site-specific recombination sites for recognition by a tyrosine recombinase, preferably wherein the tyrosine recombinase is Cre, Dre, Flp, KD, B2 or B3.

23. The cell of claim 21 or claim 22, wherein the site-specific recombination sites comprise a pair of site-specific recombination sites, and/or wherein the site-specific recombination sites comprise two or more pairs of site-specific recombination sites.

24. The cell of any one of claims 21 to 23, further comprising a gene encoding the site-specific recombinase.

25. The cell of claim 24, wherein the gene encoding the site-specific recombinase is operatively linked to a promoter, preferably wherein the promoter is temperature-sensitive, pH-sensitive, light-sensitive, or chemically-sensitive; more preferably wherein the promoter is regulated by phage lambda cI857 repressor.

26. The method of any one of claims 1 to 20, wherein the cell is as defined in any one of claims 21 to 25.

27. A polynucleotide vector comprising a gene of interest, an origin of replication, and site-specific recombination sites, wherein the site-specific recombination sites flank the gene of interest and the origin of replication, preferably wherein said polynucleotide vector comprises a nucleic acid sequence flanked by site-specific recombination sites for recognition by site-specific recombinases as defined in any one of claims 21 to 23.

28. Use of the cell of any one of claims 21 to 25, or the polynucleotide vector of claim 27, for producing a product.

29. The cell of any one of claims 21 to 25, for use in the treatment or prevention of a disease or pathology, preferably wherein the disease or pathology is cancer, metabolic disease or an immunological disorder.

30. The method, cell, polynucleotide vector, use, or cell for use, of any one of claims 1 to 29, wherein the cell is a bacterial cell, preferably wherein the bacterial cell is Escherichia sp., Bacillus sp., Lactococcus sp., Streptococcus sp., Lactobacillus sp., Corynebacterium sp., Streptomyces sp., Pseudomonas sp., Clostridium sp., Xanthomonas sp, or Enterobacteriaceae.
